SEQUENCE LISTING (72 sequences)

SEQ ID NO: 1 - DNA, 8 bp, Artificial sequence
  CRE
tgacgtca

SEQ ID NO: 2 - DNA, 5 bp, Artificial sequence
  half CRE
tgacg

SEQ ID NO: 3 - DNA, 87 bp, Artificial sequence
  cAMP-responsive promoter_1
gcaccagacagtgacgtcagctgccagatcccatggccgtcatactgtgacgtctttcagacaccccattgacgtcaatgggagaac

SEQ ID NO: 4 - DNA, 150 bp, Artificial sequence
  cAMP-responsive promoter_2
gcaccagacagtgacgtcagctgccagatcccatggccgtcatactgtgacgtctttcagacaccccattgacgtcaatgggagaacagatctggcctcggcggccaagcttagacactagagggtatataatggaagctcgacttccag

SEQ ID NO: 5 - DNA, 6 bp, Artificial sequence
  NFAT-RE
ggaaaa

SEQ ID NO: 6 - DNA, 90 bp, Artificial sequence
  NFAT-responsive promoter
ggaggaaaaactgtttcatacagaaggcgtggaggaaaaactgtttcatacagaaggcgtggaggaaaaactgtttcatacagaaggcgt

SEQ ID NO: 7 - DNA, 600 bp, Artificial sequence
  pac
atgaccgagtacaagcccacggtgcgcctcgccacccgcgacgacgtcccccgggcagtacgcaccctcgccgccgcgttcgccgactaccccgccacgcgccacaccgtcgatccagaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 8 - DNA, 1038 bp, Artificial sequence
  hph
atgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaa

SEQ ID NO: 9 - protein, 17 aa, Homo sapiens
  Homo sapiens LUCY-tag
MRPQILLLLALLTLGLA

SEQ ID NO: 10 - protein, 17 aa, Mus musculus
  Mus musculus LUCY-tag
MSHQILLLLALLTLGLA

SEQ ID NO: 11 - protein, 9 aa, Artificial sequence
  FLAG-tag
MDYKDDDDK

SEQ ID NO: 12 - protein, 20 aa, Artificial sequence
  rho-tag
MNGTEGPNFYVPFSNKTGVV

SEQ ID NO: 13 - DNA, 150 bp, Artificial sequence
  mmLucy-FLAG-rho
atgagccaccagatcctgctgctcctggccctgctgaccctaggcctggctgattacaaggacgacgacgataagatcgaattgatgaacgggaccgagggcccaaacttctacgtgcctttctccaacaagacgggcgtggtggaattc

SEQ ID NO: 14 - protein, 50 aa, Artificial sequence
  mmLucy-FLAG-rho
MSHQILLLLALLTLGLADYKDDDDKIELMNGTEGPNFYVPFSNKTGVVEF

SEQ ID NO: 15 - DNA, 588 bp, Artificial sequence
  CMV promoter
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 16 - DNA, 225 bp, Artificial sequence
  bgh terminator
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 17 - protein, 227 aa, Artificial sequence
  RTP1S  V227I
MCKSVTTDEWKKVFYEKMEEAKPADSWDLIIDPNLKHNVLSPGWKQYLELHASGRFHCSWCWHTWQSPYVVILFHMFLDRAQRAGSVRMRVFKQLCYECGTARLDESSMLEENIEGLVDNLITSLREQCYGERGGQYRIHVASRQDNRRHRGEFCEACQEGIVHWKPSEKLLEEEATTYTFSRAPSPTKSQDQTGSGWNFCSIPWCLFWATVLLLIIYLQFSFRSSI

SEQ ID NO: 18 - protein, 225 aa, Artificial sequence
  RTP2  L220R
MCTSLTTCEWKKVFYEKMEVAKPADSWELIIDPNLKPSELAPGWKQYLEQHASGRFHCSWCWHTWQSAHVVILFHMFLDRAQRAGSVRMRVFKQLCYECGTARLDESSMLEENIEGLVDNLITSLREQCYEEDGGQYRIHVASRPDSGPHRAEFCEACQEGIVHWKPSEKLLEEEVTTYTSEASKPRAQAGSGYNFLSLRWCLFWASLCLLVVYLQFSFRSPAFF

SEQ ID NO: 19 - protein, 324 aa, Homo sapiens
  hOR5A2
MAVGRNNTIVTKFILLGLSDHPQMKIFLFMLFLGLYLLTLAWNLSLIALIKMDSHLHMPMYFFLSNLSFLDICYVSSTAPKMLSDIITEQKTISFVGCATQYFVFCGMGLTECFLLAAMAYDRYAAICNPLLYTVLISHTLCLKMVVGAYVGGFLSSFIETYSVYQHDFCGPYMINHFFCDLPPVLALSCSDTFTSEVVTFIVSVVVGIVSVLVVLISYGYIVAAVVKISSATGRTKAFSTCASHLTAVTLFYGSGFFMYMRPSSSYSLNRDKVVSIFYALVIPVVNPIIYSFRNKEIKNAMRKAMERDPGISHGGPFIFMTLG

SEQ ID NO: 20 - protein, 316 aa, Homo sapiens
  hOR5A2-clone 3
MAVGRNNTIVTKFILLGLSDHPQMKIFLFMLFLGLYLLTLAWNLSLIALIKMDSHLHMPMYFFLSNLSFLDICYVSSTAPKMLSDIITEQKTISFVGCATQYFVFCGMGLTECFLLAAMAYDRYAAICNPLLYTVLISHTLCLKMVVGAYVGGFLSSFIETYSVYQHDFCGPYMINHFFCDLPPVLALSCSDTFTSEVVTFIVSVVVGIVSVLVVLISYGYIVAAVVKISSATGRTKAFSTCASHLTAVTLFYGSGFFMYMRPSSSYSLNRDKVVSIFYALVIPVVNPIIYSFRNKEIKNAMRKAMESWPRCCRSW

SEQ ID NO: 21 - DNA, 34 bp, Artificial sequence
  primer
cagagatctcgcgttgacattgattattgactag

SEQ ID NO: 22 - DNA, 27 bp, Artificial sequence
  primer
ctggtcgacagaagccatagagcccac

SEQ ID NO: 23 - DNA, 34 bp, Artificial sequence
  primer
ctggtcgaccgcgttgacattgattattgactag

SEQ ID NO: 24 - DNA, 23 bp, Artificial sequence
  primer
gcggccaagcttagacactagag

SEQ ID NO: 25 - DNA, 28 bp, Artificial sequence
  primer
cggtcatggtggctttaccaacagtacc

SEQ ID NO: 26 - DNA, 29 bp, Artificial sequence
  primer
tggtaaagccaccatgaccgagtacaagc

SEQ ID NO: 27 - DNA, 25 bp, Artificial sequence
  primer
cagtctagatcaggcaccgggcttg

SEQ ID NO: 28 - DNA, 22 bp, Artificial sequence
  primer
aacatttctctggcctaactgg

SEQ ID NO: 29 - DNA, 22 bp, Artificial sequence
  primer
attcccgatgatgagcactttc

SEQ ID NO: 30 - DNA, 32 bp, Artificial sequence
  primer
tacaggaattcatggctgtaggaaggaacaac

SEQ ID NO: 31 - DNA, 32 bp, Artificial sequence
  primer
actgcggccgcttaccatgagcgacaacaccg

SEQ ID NO: 32 - DNA, 20 bp, Artificial sequence
  primer
acaaggacgacgacgataag

SEQ ID NO: 33 - DNA, 20 bp, Artificial sequence
  primer
gatggctggcaactagaagg

SEQ ID NO: 34 - DNA, 22 bp, Artificial sequence
  primer
attaaggtacgggaggtattgg

SEQ ID NO: 35 - DNA, 22 bp, Artificial sequence
  primer
aagagtgggctatatcgaactg

SEQ ID NO: 36 - DNA, 1045 bp, Homo sapiens
  M1
  misc_feature (3)..(4): a or g or c or t
ggnncgagggcccaaacttctacgtgcctttctccaacaagacgggcgtggtggaattcatgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcytatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctgagcggccgctcgagtctagagggcccgtttaaaccgctgatcagctcgatg

SEQ ID NO: 37 - DNA, 1044 bp, Homo sapiens
  M2
  misc_feature (4)..(5): a or g or c or t
  misc_feature (1044)..(1044): a or g or c or t
cggnncgagggcccaaacttctacgtgcctttctccaacaagacgggcgtggtggaattcatgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctgagcggccgctcgagtctagagggcccgtttaaaccgctgatcagctcan

SEQ ID NO: 38 - DNA, 1036 bp, Homo sapiens
  M3
  misc_feature (5)..(5): a or g or c or t
  misc_feature (1035)..(1036): a or g or c or t
ggccnaacttctacgtgcctttctccaacaagacgggcgtggtggaattcatgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctgagcggccgctcgagtctagagggccgtttaaaccgctgatcagctcgacnn

SEQ ID NO: 39 - DNA, 1033 bp, Homo sapiens
  M4
  misc_feature (5)..(5): a or g or c or t
ggccnaamttctacgtgcctttctccaacaagacgggcgtggtggaattcatgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctgagcggccgctcgagtctagagggcccgtttaacccgctgatcagctcg

SEQ ID NO: 40 - DNA, 1041 bp, Homo sapiens
  M5
gagggccaaacttctacgtgcctttctccaacaagacgggcgtggtggaattcatgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctgagcggccgctcgagtctagagggytcgtttaaaccgctgatcagctcactgct

SEQ ID NO: 41 - DNA, 1040 bp, Homo sapiens
  M6
gagggccaaacttctacgtgcctttctccaacaagacgggcgtggtggaattcatgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctgagcggccgctcgagtctagagggcccgtttaaaccgctgatcagctcgatgc

SEQ ID NO: 42 - DNA, 1032 bp, Homo sapiens
  M7
gagggcccaaacttctacgtgcctttctccaacaagacgggcgtggtggaattcatgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctgagcggccgctcgagtctagagggcccgtttaaaccgctgatca

SEQ ID NO: 43 - DNA, 1037 bp, Homo sapiens
  M8
  misc_feature (2)..(2): a or g or c or t
anggcccaamttctacgtgcctttctccaacaagacgggcgtggtggaattcatgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctgagcggccgctcgagtctagagggcccgtttaacccgctgatcagctcgat

SEQ ID NO: 44 - DNA, 1031 bp, Homo sapiens
  M9
  misc_feature (1)..(2): a or g or c or t
nnaaamttytacgtgcctttctccaacaagacgggcgtggtggaattcatgactgggggaggaaatattacagaaatcacctatttcatcctgctgggattctcagattttcccaggatcataaaagtgctcttcactatattcctggtgatctacattacatctctggcctggaacctctccctcattgttttaataasgatggattcccmcctccatacmcccatgtatttcttcctcastaacctgtccttcatagatgtctgctatatcagctccacagtccccaagatgctctccamcctcttacaggaacakcaaactatcacttttgttggttgtattattcaktactttatcttttcaacgatgggactgagtgastcttgtctcatgacagccatggcttatgatcgttatgctgccatttgtaaccccctgctctattcatccatcatgwcacccaccctctgtgtttggatggtactgggagcctacatgactggcctcactgcttctttattccaaattggtgctttgcttcaactccacttctgtgggtcwaatgtcatcasacatttcttctgtgacatgccccaactgttaatcttgtcctgtactgacactttctttgtacaggtcatgactgctatattaaccatgttctttggratagcaagtgccctagttatcatgatatcctatggctatrttggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaasagaartgctgctgagcggccgctcgagtctagagggcccgtttaacccgctgatcagctcac

SEQ ID NO: 45 - DNA, 1037 bp, Homo sapiens
  E1
  misc_feature (4)..(4): a or g or c or t
ggcnaaamttctacgtgcctttctccaacaagacgggcgtggtggaattcatgtccaacgcctccctackgacagcgttcatcctcacgggccttccccatgccccagggctggacscccccctctttggaatcttcctggtggtttacktgctcactgtgctggggaacctcctcatcckgctggtgatcagggtggattctcacctccacacccccatgtactacttcctcaccaacctgtccttcattgacwtgtggttctccactgtcacggwgcccaaaatgctgatgaccttggtgtccccaarcggcaggactatctccwtcmacagctgcgtggctcagctctattttttccacttcctggggagcaccgagtgtttcctctacacagtcatgtcctatgatcgctacctggccatcakttacccgctcaggtacaccaacatgatgactgggcgctcgtgtgccctcctggccaccggcacttggctcagtggctctctgcactctgctgtccagaccatattgactttccatttgccctactgtggacccaaccagatccagcactacttctgtgacscaccgcccatcctgaaactggcctgtgcakacacctcagccaacgagatggtcatctttgtgaatattgggctastggcctcgggctgctttrtcctgatagtgctgycctatgtgtccatcgtctgttccatcctgcggatccgcacctcagaggggaggcacagagcctttcagacctgtgcctcccactgtatcgtggtcctttgcttctttggccctggtcttttcatttacctgaggccaggctccagggacgccttgcatggggttgtggccgttttctacaccacgctgactcctcttttcaaccctgwtgtgtacaccctgasaaacaaggaggtaaagaaagctctgttgaagctgaaaaatgsgtcagtatttgctcaggatgaataggcggycgctcgagtctagagggyccgtttaaaccgctgatcagcctcgayg

SEQ ID NO: 46 - DNA, 55 bp, Homo sapiens
  E2
  misc_feature (52)..(52): a or g or c or t
acttctacgtgcctttctccaacaagacgggcgtggtggaattcatgwccancgc

SEQ ID NO: 47 - DNA, 1040 bp, Homo sapiens
  E3
  misc_feature (1010)..(1010): a or g or c or t
gggccaaacttctacgtgcctttctccaacaagacgggcgtggtggaattcatgtccaacgcctccctactgacagcgttcatcctcacgggccttccccatgccccagggctggacscccccctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcaggactatctccttccacagctgcgtggctcagctctattttttccacttcctggggagcaccgagtgtttcctctacacagtcatgtcctatgatcgctacctggccatcagttacccgctcaggtacaccaacatgatgactgggcgctcgtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcasccaacgagatggtcatctttgtgaatattgggctagtggcctcgggctgctttgtcctgatagtgctgtcctatgtgtccatcgtctgttccatcctgcggatccgcacctcagaggggaggcacagagcctttcagacctgtgcctcccactgtatcgtggtcctttgcttctttggccctggtcttttcatttacctgaggccaggctccagggacgccttgcatggggttgtggccgttttctacaccacgctgactcctcttttcaaccctgttgtgtacaccctgagaaacaaggaggtaaagaaagctctgttgaagctgaaaaatgggtcagtatttgctcagggtgaataggcggccgctcgagtctagagggncgtttaacccgctgatcagctcgactgcta

SEQ ID NO: 48 - DNA, 1029 bp, Homo sapiens
  E4
  misc_feature (1)..(3): a or g or c or t
nnncttctacgtgcctttctccaacaagacgggcgtggtggaattcatgtccracgcctccctactgacagcgttcatcctcacgggccttccccatgccccagggctggacgcccccctctttggaatcttcctggtggtttasgtgctcactgtgctggggaacctcctcatcctgctggtgatcrgggtggattctcacctccacacccccatgtactacttcctcaccaacctgtccttcattgacatgtggttctccactgtcacggtgcccaaaatgctgatgaccttggtgtccccaagcggcargactatctccttccacagctgcgtggctcakctctattttttccacttcctggggagcaccgagwgtttcctctacacagtcatgtcctatgatcgctacctggccatcagttacccgctcaggtacaccaacatgatgactgggcgctcgtgtgccctcctggccaccggcacttggctcagtggctctctgcactctgctgtccakaccatattgactttccatttgccctactgtggacccaaccasatccrgcactacttctgtgacgcaccrcccatcctgaaactggcctgtgcasacacctcasccaacgagatggtcatctttgtgaatattgggctastggcctcgggctgctttgtcctgatartgctgtcctatgtgtccatcgtctgttccatcctgcggatccgcacctcagaggggaggcacagagcctttcagacctgtgcctcccactgtatcgtggtcctttgcttctttggccctggtcttttcatttacctgaggccaggctccagggacgccttgcatggcgttgtggccgttwtctacaccacgctgactcctcttttcaaccctgwtgtgtacaccctgagaaacaaggaggtaaagaaagctctgttgaarctgaaaaatgggtcagtatttgctcagggtgaataggcggccgctcgagtctagagggcccgtttaaacccgctgatcacctc

SEQ ID NO: 49 - DNA, 1036 bp, Homo sapiens
  E5
ggcccaacttctacgtgcctttctccaacaagacgggcgtggtggaattcatgtccaacgcctccctactgacagcgttcatcctcacgggccttccccatgccccagggctggacgcccccctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcaggactatctccttccacagctgcgtggctcagctctattttttccacttcctggggagcaccgagtgtttcctctacacagtcatgtcctatgatcgctacctggccatcagttacccgctcaggtacaccaacatgatgactgggcgctcgtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgagatggtcatctttgtgaatattgggctagtggcctcgggctgctttgtcctgatagtgctgtcctatgtgtccatcgtctgttccatcctgcggatccgcacctcagaggggaggcacagagcctttcagacctgtgcctcccactgtatcgtggtcctttgcttctttggccctggtcttttcatttacctgaggccaggctccagggacgccttgcatggggttgtggccgttttctacaccacgctgactcctcttttcaaccctgttgtgtacaccctgagaaacaaggaggtaaagaaagctctgttgaagctgaaaaatgggtcagtatttgctcagggtgaataggcggccgctcgagtctagagggcccgtttaaacccgctgatcagctcrct

SEQ ID NO: 50 - DNA, 1034 bp, Homo sapiens
  E6
  misc_feature (3)..(3): a or g or c or t
  misc_feature (171)..(171): a or g or c or t
  misc_feature (1014)..(1014): a or g or c or t
ganggcccaaacttctacgtgcctttctccaacaagacgggcgtggtggaattcatgtccaacgcctccctactgacagcgttcatcctcacgggccttccccatgccccagggctggacscccccctctttggaatcttcctggtggtttacgtgctcactgtgctgggngaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcaggactatctccttccacagctgcgtggctcagctctattttttccacttcctggggagcaccgagtgtttcctctacacagtcatgtcctatgatcgctacctggccatcagttacccgctcaggtacaccaacatgatgactgggcgctcgtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcasacacctcagccaacgagatggtcatctttgtgaatattgggctagtggcctcgggctgctttgtcctgatagtgctgtcctatgtgtccatcgtctgttccatcctgcggatccgcacctcagaggggaggcacagagcctttcagacctgtgcctcccactgtatcgtggtcctttgcttctttggccctggtcttttcatttacctgaggccaggctccagggacgccttgcatggggttgtggccgttttctacaccacgctgactcctcttttcaaccctgttgtgtacaccctgagaaacaaggaggtaaagaaagctctgttgaagctgaaaaatgggtcagtatttgctcagggtgaataggcggccgctcgagtctagagggncgtttaacccgctgatcagc

SEQ ID NO: 51 - DNA, 1044 bp, Homo sapiens
  E7
gagggcccaaacttctacgtgcctttctccaacaagacgggcgtggtggaattcatgtccaacgcctccctactgacagcgttcatcctcacgggccttccccatgccccagggctggacgcccccctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcaggactatctccttccacagctgcgtggctcagctctattttttccacttcctggggagcaccgagtgtttcctctacacagtcatgtcctatgatcgctacctggccatcagttacccgctcaggtacaccaacatgatgactgggcgctcgtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgagatggtcatctttgtgaatattgggctagtggcctcgggctgctttgtcctgatagtgctgtcctatgtgtccatcgtctgttccatcctgcggatccgcacctcagaggggaggcacagagcctttcagacctgtgcctcccactgtatcgtggtcctttgcttctttggccctggtcttttcatttacctgaggccaggctccagggacgccttgcatggggttgtggccgttttctacaccacgctgactcctcttttcaaccctgttgtgtacaccctgagaaacaaggaggtaaagaaagctctgttgaagctgaaaaatgggtcagtatttgctcagggtgaataggcggccgctcgagtctagagggcccgtttaaacccgctgatcagctcgatggct

SEQ ID NO: 52 - DNA, 1023 bp, Homo sapiens
  E8
  misc_feature (1)..(3): a or g or c or t
  misc_feature (1005)..(1005): a or g or c or t
nnncttctacgtgcctttctccaacaagacgggcgtggtggaattcatgtccaacgcctccctactgacagcgttcatcctcacgggccttccccatgccccagggctggacscccccctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcaggactatctccttccacagctgcgtggctcagctctattttttccacttcctggggagcaccgagtgtttcctctacacagtcatgtcctatgatcgctacctggccatcagttacccgctcaggtacaccaacatgatgactgggcgctcgtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcasacacctcagccaacgagatggtcatctttgtgaatattgggctagtggcctcgggctgctttgtcctgatagtgctgtcctatgtgtccatcgtctgttccatcctgcggatccgcacctcagaggggaggcacagagcctttcagacctgtgcctcccactgtatcgtggtcctttgcttctttggccctggtcttttcatttacctgaggccaggctccagggacgccttgcatggggttgtggccgttttctacaccacgctgactcctcttttcaaccctgttgtgtacaccctgagaaacaaggaggtaaagaaagctctgttgaagctgaaaaatgggtcagtatttgctcagggtgaataggcggccgctcgagtctagagggncgtttaaacccgctgatc

SEQ ID NO: 53 - DNA, 1029 bp, Homo sapiens
  E9
  misc_feature (1)..(3): a or g or c or t
nnnaacttctacgtgcctttctccaacaagacgggcgtggtggaattcatgtccaacgcctccctactgacagcgttcatcctcacgggccttccccatgccccagggctggacgcccccctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcaggactatctccttccacagctgcgtggctcagctctattttttccacttcctggggagcaccgagtgtttcctctacacagtcatgtcctatgatcgctacctggccatcagttacccgctcaggtacaccaacatgatgactgggcgctcgtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgagatggtcatctttgtgaatattgggctagtggcctcgggctgctttgtcctgatagtgctgtcctatgtgtccatcgtctgttccatcctgcggatccgcacctcagaggggaggcacagagcctttcagacctgtgcctcccactgtatcgtggtcctttgcttctttggccctggtcttttcatttacctgaggccaggctccagggacgccttgcatggggttgtggccgttttctacaccacgctgactcctcttttcaaccctgttgtgtacaccctgagaaacaaggaggtaaagaaagctctgttgaagctgaaaaatgggtcagtatttgctcagggtgaataggcggccgctcgagtctagagggcccgtttaaaccgctgatcagct

SEQ ID NO: 54 - DNA, 1037 bp, Homo sapiens
  V1
ggccaacttctacgtgcctttctccaacaagacgggcgtggtggaattcatgtccaacgccagcctcgtgacagcattcatcctcacaggccttccccatgccccagggctggacgccctcctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcagggctatctccttccacagctgcgtggctcagctctattttttccacttcctggggagcaccgagtgtttcctctacacagtcatgtcctatgatcgctacttggccatcagttacccgctcaggtacaccagcatgatgagtgggagcaggtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgtgatggtcatctttgtggacattgggatagtggcctcaggctgctttgtcctgatagtgctgtcctatgtgtccatcgtctgttccatcctgcgcatccgcacctcagatgggaggcgcagagcctttcagacctgtgcctcccactgtattgtggtcctttgcttctttgttccctgtgttgtcatttatctgaggccaggctccatggatgccatggatggagttgtggccattttctacactgtgctgacgccccttctcaaccctgttgtgtacaccctgagaaacaaggaggtgaagaaagctgtgttgaaacttagagacaaagtagcacatcctcagaggaaataagcggccgctcgagtctagagggcccgtttaaacccgctgatcagcctcgact

SEQ ID NO: 55 - DNA, 1046 bp, Homo sapiens
  V2
  misc_feature (3)..(4): a or g or c or t
ggnncgagggccaaacttctacgtgcctttctccaacaagacgggcgtggtggaattcatgtccaacgccagcctcgtgacagcattcatcctcacaggccttccccatgccccagggctggacgccctcctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcagggctatctccttccacagctgcgtggctcagctctattttttccacttcctggggagcaccgagtgtttcctctacacagtcatgtcctatgatcgctacttggccatcagttacccgctcaggtacaccagcatgatgagtgggagcaggtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgtgatggtcatctttgtggacattgggatagtggcctcaggctgctttgtcctgatagtgctgtcctatgtgtccatcgtctgttccatcctgcgcatccgcacctcagatgggaggcgcagagcctttcagacctgtgcctcccactgtattgtggtcctttgcttctttgttccctgtgttgtcatttatctgaggccaggctccatggatgccatggatggagttgtggccattttctacactgtgctgacgccccttctcaaccctgttgtgtacaccctgagaaacaaggaggtgaagaaagctgtgttgaaacttagagacaaagtagcacatcctcagaggaaataagcggccgctcgagtctagagggcccgtttaaacccgctgatcagcctcgact

SEQ ID NO: 56 - DNA, 1036 bp, Homo sapiens
  V3
  misc_feature (3)..(5): a or g or c or t
ggnnnacttctacgtgcctttctccaacaagacgggcgtggtggaattcatgtccaacgccwscctcgtgacagcattcatcctcacaggccttccccatgccccagggctggacgccctcctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcagggctatctccttccacagctgcgtggctcagctctattttttccacttcctggggagcaccgagtgtttcctctacacagtcatgtcctatgatcgctacttggccatcagttacccgctcaggtacaccarcatgatgagtgggagcaggtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgtgatggtcatctttgtgracattgggatagtggcctcaggctgctttgtcctgatagtgctgtcctatgtgtccatcgtctgttccatcctgcgcatccgcacctcagatgggaggcgcagagcctttcagacctgtgcctcccactgtattgtggtcctttgcttctttgttccctgtgttgtcatttatctgaggccaggctccatggatgccatggatggagttgtggccattttctacactgtgctgacgcctcttctcaaccctgttgtgtacaccctgagaaacaaggaggtgaagaaagctgtgttgaaacttagagacraagtagcacatcctcagaggaaataagcggccgctcgagtctagagggcccgtttaaacccgctgatcagcctcgac

SEQ ID NO: 57 - DNA, 1042 bp, Homo sapiens
  V5
  misc_feature (2)..(4): a or g or c or t
gnnnacttctacgtgcctttctccaacaagacgggcgtggtggaattcatgtccaacgcctccctmctgacagcrttcatcctcacrggccttccccatgccccagggctggacgcccycctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcaggactatctccttccacagctgcgtggctcagctctattttttccacttcctggggagcaccgagtgtttcctctacacagtcatgtcctatgatcgctacytggccatcagttacccgctcaggtacaccaacatgatgactgggmgcwsgtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgagatggtcatctttgtgaayattgggmtagtggcctcrggctgctttgtcctgatagtgctgtcctatgtgtccatcgtctgttccatcctgcgsatccgcacctcagatgggaggcacagagcctttcagacctgtgcctcccactgtatcgtggtcctttgcttctttgktcctggtsttttcatttayctgaggccaggctccatggaygccwtgcatggagttgtggccgttttctacacygygctgactcctcttctcaaccctgttgtgtacaccctgagaaacaaggaggtraagaaagctstgttgaaactgaaagaygrrtyagyayatgctcagrgtgaataagcggccgctcgagtctagagggcccgtttaaacccgctgatcacctcgatggctcagt

SEQ ID NO: 58 - DNA, 1033 bp, Homo sapiens
  V6
  misc_feature (1)..(3): a or g or c or t
nnncttctacgtgcctttctccaacaagacgggcgtggtggaattcatgtccaacgccagcctcgtgacagcattcatcctcacaggccttccccatgccccagggctggacgccctcctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcagggctatctccttccacagctgcgtggctcagctctattttttccacttcctggggagcaccgagtgtttcctctacacagtcatgtcctatgatcgctacttggccatcagttacccgctcaggtacaccagcatgatgagtgggagcaggtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgtgatggtcatctttgtggacattgggatagtggcctcaggctgctttgtcctgatagtgctgtcctatgtgtccatcgtctgttccatcctgcgcatccgcacctcagatgggaggcgcagagcctttcagacctgtgcctcccactgtattgtggtcctttgcttctttgttccctgtgttgtcatttatctgaggccaggctccatggatgccatggatggagttgtggccattttctacactgtgctgacgccccttctcaaccctgttgtgtacaccctgagaaacaaggaggtgaagaaagctgtgttgaaacttagagacaaagtagcacatcctcagaggaaataagcggccgctcgagtctagagggcccgtttaaacccgctgatcacctcgatg

SEQ ID NO: 59 - DNA, 1035 bp, Homo sapiens
  V7
  misc_feature (2)..(3): a or g or c or t
cnnaacttctacgtgcctttctccaacaagacgggcgtggtggaattcatgtccaacgccagcctcgtgacagcattcatcctcacaggccttccccatgccccagggctggacgccctcctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcagggctatctccttccacagctgcgtggctcagctctattttttccacttcctggggagcaccgagtgtttcctctacacagtcatgtcctatgatcgctacttggccatcagttacccgctcaggtacaccagcatgatgagtgggagcaggtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgtgatggtcatctttgtggacattgggatagtggcctcaggctgctttgtcctgatagtgctgtcctatgtgtccatcgtctgttccatcctgcgcatccgcacctcagatgggaggcgcagagcctttcagacctgtgcctcccactgtattgtggtcctttgcttctttgttccctgtgttgtcatttatctgaggccaggctccatggatgccatggatggagttgtggccattttctacactgtgctgacgccccttctcaaccctgttgtgtacaccctgagaaacaaggaggtgaagaaagctgtgttgaaacttagagacaaagtagcacatcctcagaggaaataagcggccgctcgagtctagagggcccgtttaaacccgctgatcacctcgatg

SEQ ID NO: 60 - DNA, 1045 bp, Homo sapiens
  V8
  misc_feature (1)..(3): a or g or c or t
nnnacttctacgtgcctttctccaacaagacgggcgtggtggaattcatgtccaacgccagcctcgtgacagcattcatcctcacaggccttccccatgccccagggctggacgccctcctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcagggctatctccttccacagctgcgtggctcagctctattttttccacttcctggggagcaccgagtgtttcctctacacagtcatgtcctatgatcgctacttggccatcagttacccgctcaggtacaccagcatgatgagtgggagcaggtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgtgatggtcatctttgtggacattgggatagtggcctcaggctgctttgtcctgatagtgctgtcctatgtgtccatcgtctgttccatcctgcgcatccgcacctcagatgggaggcgcagagcctttcagacctgtgcctcccactgtattgtggtcctttgcttctttgttccctgtgttgtcatttatctgaggccaggctccatggatgccatggatggagttgtggccattttctacactgtgctgacgccccttctcaaccctgttgtgtacaccctgagaaacaaggaggtgaagaaagctgtgttgaaacttagagacaaagtagcacatcctcagaggaaataagcggccgctcgagtctagagggcccgtttaaacccgctgatcagcctcgatggctcatttgt

SEQ ID NO: 61 - DNA, 1042 bp, Homo sapiens
  V9
  misc_feature (3)..(3): a or g or c or t
  misc_feature (5)..(5): a or g or c or t
ggncnacttctacgtgcctttctccaacaagacgggcgtggtggaattcatgtccaacgccagcctcgtgacagcattcatcctcacaggccttccccatgccccagggctggacgccctcctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcagggctatctccttccacagctgcgtggctcagctctattttttccacttcctggggagcaccgagtgtttcctctacacagtcatgtcctatgatcgctacttggccatcagttacccgctcaggtacaccagcatgatgagtgggagcaggtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgtgatggtcatctttgtggacattgggatagtggcctcaggctgctttgtcctgatagtgctgtcctatgtgtccatcgtctgttccatcctgcgcatccgcacctcagatgggaggcgcagagcctttcagacctgtgcctcccactgtattgtggtcctttgcttctttgttccctgtgttgtcatttatctgaggccaggctccatggatgccatggatggagttgtggccattttctacactgtgctgacgccccttctcaaccctgttgtgtacaccctgagaaacaaggaggtgaagaaagctgtgttgaaacttagagacaaagtagcacatcctcagaggaaataagcggccgctcgagtctagagggcccgtttaaacccgctgatcagcctcgactggctc

SEQ ID NO: 62 - protein, 43 aa, Homo sapiens
  C-terminus OR5A2 clone 3
VVSIFYALVIPVVNPIIYSFRNKEIKNAMRKAMESWPRCCRSW

SEQ ID NO: 63 - DNA, 28 bp, Artificial sequence
  primer
gacaaaggcatggtggctttaccaacag

SEQ ID NO: 64 - DNA, 393 bp, Artificial sequence
  bsd
atgcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 65 - protein, 130 aa, Artificial sequence
  bsd
MPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 66 - DNA, 35 bp, Artificial sequence
  primer
gtaaagccaccatgcctttgtctcaagaagaatcc

SEQ ID NO: 67 - DNA, 31 bp, Artificial sequence
  primer
ccgactctagattagccctcccacacataac

SEQ ID NO: 68 - DNA, 36 bp, Artificial sequence
  primer
atcaggccggccgccccgactctagattagccctcc

SEQ ID NO: 69 - protein, 51 aa, Homo sapiens
  C-terminus hOR5A2
VVSIFYALVIPVVNPIIYSFRNKEIKNAMRKAMERDPGISHGGPFIFMTLG

SEQ ID NO: 70 - DNA, 936 bp, Homo sapiens
  OR5AN1
atgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctga

SEQ ID NO: 71 - DNA, 936 bp, Homo sapiens
  OR10G7
atgtccaacgcctccctactgacagcgttcatcctcacgggccttccccatgccccagggctggacgcccccctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcaggactatctccttccacagctgcgtggctcagctctattttttccacttcctggggagcaccgagtgtttcctctacacagtcatgtcctatgatcgctacctggccatcagttacccgctcaggtacaccaacatgatgactgggcgctcgtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgagatggtcatctttgtgaatattgggctagtggcctcgggctgctttgtcctgatagtgctgtcctatgtgtccatcgtctgttccatcctgcggatccgcacctcagaggggaggcacagagcctttcagacctgtgcctcccactgtatcgtggtcctttgcttctttggccctggtcttttcatttacctgaggccaggctccagggacgccttgcatggggttgtggccgttttctacaccacgctgactcctcttttcaaccctgttgtgtacaccctgagaaacaaggaggtaaagaaagctctgttgaagctgaaaaatgggtcagtatttgctcagggtgaatag

SEQ ID NO: 72 - DNA, 936 bp, Homo sapiens
  OR10G4
atgtccaacgccagcctcgtgacagcattcatcctcacaggccttccccatgccccagggctggacgccctcctctttggaatcttcctggtggtttacgtgctcactgtgctggggaacctcctcatcctgctggtgatcagggtggattctcacctccacacccccatgtactacttcctcaccaacctgtccttcattgacatgtggttctccactgtcacggtgcccaaaatgctgatgaccttggtgtccccaagcggcagggctatctccttccacagctgcgtggctcagctctattttttccacttcctggggagcaccgagtgtttcctctacacagtcatgtcctatgatcgctacttggccatcagttacccgctcaggtacaccagcatgatgagtgggagcaggtgtgccctcctggccaccggcacttggctcagtggctctctgcactctgctgtccagaccatattgactttccatttgccctactgtggacccaaccagatccagcactacttctgtgacgcaccgcccatcctgaaactggcctgtgcagacacctcagccaacgtgatggtcatctttgtggacattgggatagtggcctcaggctgctttgtcctgatagtgctgtcctatgtgtccatcgtctgttccatcctgcgcatccgcacctcagatgggaggcgcagagcctttcagacctgtgcctcccactgtattgtggtcctttgcttctttgttccctgtgttgtcatttatctgaggccaggctccatggatgccatggatggagttgtggccattttctacactgtgctgacgccccttctcaaccctgttgtgtacaccctgagaaacaaggaggtgaagaaagctgtgttgaaacttagagacaaagtagcacatcctcagaggaaataa

(19) United States
(12) Patent Application Publication
(10) Pub. No.: US 2024/0426807 A1
(43) Pub. Date: Dec. 26, 2024

(54) SCREENING SYSTEM

(71) Applicant: Givaudan SA, VERNIER (CH)

(72) Inventors: Roger EMTER, Zurich (CH); Andreas NATSCH, Uetikon (CH)

(21) Appl. No.: 18/570,418

(22) PCT Filed: Jun. 21, 2022
(86) PCT No.: PCT/EP2022/066779

§ 371 Date: Dec. 14, 2023

(30) Foreign Application Priority Data
Jun. 21, 2021 (GB) 2108867.9

(51) Int. Cl. G01N 33/50; C12N 9/22; C12N 15/11; C12N 15/90

(52) U.S. Cl. CPC G01N 33/5008; C12N 9/22; C12N 15/11; C12N 15/907; C12N 2310/20

(57) ABSTRACT

Described herein are nucleic acid constructs comprising a promoter and/or enhancer sequence operably linked to a nucleic acid sequence encoding a polypeptide, wherein said promoter and/or enhancer is inducible by an olfactory receptor, cells and populations comprising said constructs, and methods for expressing olfactory receptors and for identifying novel olfactory receptors and receptor-ligand interactions.

### FIELD

Aspects and embodiments described herein relate to the field of olfactory receptors and to nucleic acid constructs, host cells, and methods for expressing olfactory receptors and for identifying novel olfactory receptors and receptor-ligand interactions.

### BACKGROUND

Olfactory or odorant receptors (ORs) are expressed in olfactory sensory neurons of the olfactory epithelium and are responsible for the detection of odorants. Olfactory receptors belong to the G protein-coupled receptor superfamily (GPCRs). Activation of an OR by an odorant (ligand) activates the olfactory-specific G protein which in turn promotes the production of cyclic AMP (cAMP) via a type III adenylate cyclase. The increased levels of intracellular cAMP results in the opening of cyclic nucleotide-gated ion channels which allow calcium ions to enter into the cell, depolarizing the olfactory sensory neuron and triggering an action potential which carries the information to the brain.

The human genome encodes approximately 400 different functional olfactory receptors. A specific olfactory receptor may be activated by more than one ligand molecule and a specific ligand molecule may activate multiple olfactory receptors, which creates a highly complex interaction network between the OR and ligand repertoires. Elucidation of said interactions can allow for the discovery of novel flavour and fragrance ingredients, or compounds such as odor enhancers that are more sustainable and/or easier to produce than currently used compounds.

Efficient screening of olfactory receptors requires their expression in cultured cell lines, which generally involves the introduction of an olfactory receptor gene into a cell followed by its stable or transient overexpression. Functional heterologous olfactory receptor expression utilizing the expression systems currently available in the art generally requires co-expression of accessory proteins of the receptor transporting protein (RTP) family, namely RTP1S and RTP2 (Yu et al. (2017) PLOS One 12 (6): e0179067), which are normally expressed in the olfactory sensory neurons and facilitate OR trafficking to the cell-surface membrane. However, more than half of the known olfactory receptors cannot be functionally expressed utilizing currently available nucleic acid constructs, cell lines, and methods, resulting in a presently limited coverage of the available receptor-ligand space, with multiple receptors not having identified ligands (orphan receptors), and limited industrial application of said methods. Therefore, there is a need for improved nucleic acid constructs, cell lines, and methods for expressing olfactory receptors and for identifying novel cognate receptor-ligand interactions.

Classical OR screening assays rely on approaches wherein a clonal population of cells generally receives one receptor and/or accessory molecule at a time and is then tested for functional activation by various ligands. Said assays further generally involve the co-expression of a luciferase gene operably linked to a cAMP-inducible promoter (Saito et al. (2004) Cell 119 (5): 679-691), which is used as a reporter gene. The activation of the olfactory receptor and subsequent increase in intracellular cAMP results in expression of luciferase.

Cleavage of luciferin by luciferase results in the emission of light which can then be detected and quantified. Classical OR screening assays are limited by the number of olfactory receptors, accessory molecules, and/or ligands that can be screened at a time, are time-consuming and cumbersome, and are not compatible with high-throughput screening and selection methods such as screening of libraries of volatile flavour and fragrance compounds. Therefore, there is still a need for improved screening assays for ORs without the aforementioned drawbacks.

### SUMMARY

An aspect of the invention relates to a method for selecting for a cell expressing a functional olfactory receptor and/or for accessory molecules needed for said functional expression in a cell, said method comprising the following steps:
A) Providing cells, wherein said cells comprise a nucleic acid construct comprising a promoter and/or enhancer sequence operably linked to a nucleic acid sequence encoding a polypeptide, wherein said encoded polypeptide confers resistance to an antibiotic, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor,
B) Culturing said cells in the presence of the ligand of said olfactory receptor,
C1) Selecting for cells functionally expressing the olfactory receptor by culturing them in the presence of the antibiotic and the ligand.

Another aspect of the invention relates to a method for selecting for a cell expressing a functional olfactory receptor and/or for accessory molecules needed for said functional expression in a cell, said method comprising the following steps:
A) Applying a mutagenesis step to cells, wherein said cells comprise a nucleic acid construct comprising a promoter and/or enhancer sequence operably linked to a nucleic acid sequence encoding a polypeptide, wherein said encoded polypeptide confers resistance to an antibiotic, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor,
B) Culturing the mutated cells in the presence of the ligand of said olfactory receptor,
C1) Selecting for mutated cells functionally expressing the olfactory receptor by culturing them in the presence of the antibiotic and the ligand.

In some embodiments, the mutagenesis step is carried out using insertional mutagenesis, wherein a nucleic acid sequence is inserted in the genome of the cells using plasmids, linearized DNA sequences, transposons, retroviruses, lentiviruses or CRISPR-Cas mediated recombination. In some embodiments, the inserted nucleic acid sequence comprises an enhancer and/or promoter sequence suitable for activation of expression of endogenous genes. In some embodiments, the insertion site of the inserted nucleic acid sequence in the selected cells is mapped and/or identified. In some embodiments, the mutagenesis step is carried out using CRISPR-Cas-mediated mutagenesis, using CRISPR interference (CRISPRi) or CRISPR activation (CRISPRa).

Another aspect of the invention relates to a method for identifying an olfactory receptor binding to a given ligand, said method comprising the following steps:
A) Providing a heterogeneous population of cells, wherein said cells comprise a nucleic acid construct comprising a promoter and/or enhancer sequence operably linked to a nucleic acid sequence encoding a polypeptide, wherein said encoded polypeptide confers resistance to an antibiotic, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor, wherein the olfactory receptor encoded by the nucleic acid molecule comprised in said second nucleic acid construct comprised in at least one of the cells is distinct from the olfactory receptor encoded by the nucleic acid molecule comprised in the second nucleic acid construct in at least one of the other cells of said population,
B) Culturing said population of cells in the presence of said given ligand,
C1) Selecting for cells functionally expressing the olfactory receptor binding to said given ligand by culturing them in the presence of the antibiotic and the ligand,
D) Determining the nucleotide sequence encoding the receptor in the selected cells.

In some embodiments, the methods according to the invention are such that step C1) additionally comprises a sub-culturing step, wherein cells with improved functional expression of the olfactory receptor are enriched in a culture.

In some embodiments, the methods according to the invention are such that the nucleic acid sequence encoding the polypeptide that confers resistance to an antibiotic is a puromycin-N-acetyltransferase gene or a blasticidin-S deaminase gene.

Another aspect of the invention relates to a nucleic acid construct comprising a promoter and/or enhancer sequence operably linked to a nucleic acid sequence encoding a polypeptide conferring resistance to an antibiotic, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE). In some embodiments, the nucleic acid sequence encoding the polypeptide that confers resistance to an antibiotic is a puromycin-N-acetyltransferase gene or a blasticidin-S deaminase gene.

Another aspect of the invention relates to a cell comprising the nucleic acid construct as defined above and comprising a second nucleic acid construct comprising a nucleic acid molecule encoding an olfactory receptor, preferably wherein said nucleic acid constructs are fused so as to constitute a single nucleic acid construct. In some embodiments, the cell is a eukaryotic cell, preferably a human cell.

Another aspect of the invention relates to a population of cells as defined above, wherein the olfactory receptor encoded by the nucleic acid molecule comprised in the second nucleic acid construct comprised in at least one of the cells is distinct from the olfactory receptor encoded by the nucleic acid molecule comprised in the second nucleic acid construct in at least one of the other cells within said population, defining a pool of cells expressing distinct olfactory receptors. In some embodiments, the population of cells is such that at least one olfactory receptor is functionally expressed in said population of cells.

Another aspect of the invention relates to an olfactory receptor whose amino acid sequence comprises an amino acid sequence having at least 60% identity or similarity with SEQ ID NO: 62, preferably wherein SEQ ID NO: 62 is located at the C-terminus of the olfactory receptor. Another aspect of the invention relates to an olfactory receptor, preferably whose amino acid sequence comprises an amino acid sequence having at least 60% identity or similarity with SEQ ID NO: 62, more preferably wherein SEQ ID NO: 62 is located at the C-terminus of the olfactory receptor, whose amino acid sequence comprises, consists essentially of, or consists of an amino acid sequence having at least 60% identity or similarity with SEQ ID NO: 20.

### DESCRIPTION

The present invention provides nucleic acid constructs, cells, and methods useful in functional expression of olfactory receptors (ORs) which are difficult to express using conventional approaches and in the identification of novel cognate receptor-ligand pairs. The invention further provides olfactory receptors with improved functional expression and/or improved accessory molecules. Nucleic acid constructs, cells, and methods described herein exhibit at least one, at least two, at least three, at least four, at least five, or all of the following benefits over known nucleic acid constructs, cells and methods:
Enablement of functional expression of olfactory receptors otherwise not possible using conventional approaches.
Enablement of selection or screening and sorting out of cells functionally expressing olfactory receptors and/or accessory molecules
Enablement of identification of improved accessory molecules and/or genetic and/or epigenetic modifications required for functional or improved functional expression of olfactory receptors
Enablement of identification of novel cognate receptor-ligand pairs
Increased olfactory receptor, accessory molecule, and ligand screening throughput capacity

As also demonstrated in the Examples section herein, a significant improvement over conventional approaches is expected from the application of the nucleic acid constructs, cells, and methods of the invention. Accordingly, the aspects and embodiments of the present invention as described herein solve at least some of the problems and needs as discussed herein.

### Nucleic Acid Construct

In a first aspect, the invention provides a nucleic acid construct comprising a promoter and/or enhancer sequence operably linked to a nucleic acid sequence encoding a polypeptide, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE). In some embodiments, the promoter and/or enhancer comprises one or more copies of a cAMP responsive element (CRE). In some embodiments, the promoter and/or enhancer comprises one or more copies of a half CRE. In some embodiments, the promoter and/or enhancer comprises one or more copies of a NFAT responsive element (NFAT-RE). A definition of a cAMP responsive element (CRE), a half CRE, and an NFAT responsive element (NFAT-RE) is provided elsewhere herein.

The term “olfactory receptor” or “odorant receptor” (OR) as used herein has its customary meaning as ordinarily understood by the skilled person in view of this disclosure. It refers to receptors pertaining to the seven-transmembrane-domain G protein-coupled receptor superfamily (GPCRs), which are typically expressed in the cell membrane of olfactory receptor neurons. The predicted seven-transmembrane (TM) domains TM I to TM VII are connected by three predicted internal (IC) loop domains (IC I to IC III), and three predicted external (EC) loop domains (EC I to EC III). ORs typically comprise olfactory receptor-specific amino acid motifs. Examples of such motifs are a N-MAYDRYVAIC-C motif overlapping TM III and IC II, a N-FSTCSSH-C motif overlapping IC III and TM VI, a N-PMLNPFIY-C motif in TM VII as well as three conserved C residues in EC II, and the presence of highly conserved GN residues in TM I, discussed in Zhang and Firestein (2002) Nature Neurosci 5 (2): 124-33, and Malnic et al. (2004) PNAS 101 (8): 2584-9, all of which are incorporated herein by reference in their entireties. Mammalian and human olfactory receptors are discussed in publications such as Mainland et al. (2015) Sci Data 2:150002, incorporated herein by reference in its entirety, and in publicly available databases, such as the HORDE (The Human Olfactory Data Explorer) database maintained by the Weizmann Institute of Science, described in Olender et al. (2013) Methods Mol Biol 1003:23-38, incorporated herein by reference in its entirety.

Activation of an olfactory receptor by an odorant (ligand) in an olfactory neuron typically activates the olfactory-specific G protein (Gα_{olf}) which in turn promotes the production of cyclic adenosine monophosphate (cAMP) via a type III adenylate cyclase. The increased levels of intracellular cAMP induces the entry of calcium into the olfactory receptor neurons via the opening of a cAMP-gated cation channel. The entry of calcium causes the opening of another channel causing the exit of chloride ions, triggering an action potential leading to a signal to the respective area of the brain. An olfactory receptor may interact with multiple ligands and a ligand may activate multiple olfactory receptors.

As used herein, the term “expression” of a DNA molecule by a cell includes any step involved in the production of a polypeptide by a cell including, but not limited to, transcription, post-transcriptional modification, translation, post-translational modification, transport to a cellular membrane, and secretion. Expression may be assessed by any method known to a person of skill in the art. For example, expression may be assessed by measuring the levels of gene expression in a transduced cell on the level of the mRNA or the protein by standard assays known to a person of skill in the art, such as qPCR, RNA sequencing, Northern blot analysis, Western blot analysis, mass spectrometry analysis of protein-derived peptides or ELISA.

As used herein, the term “functional expression” refers to the production of a polypeptide by a cell wherein the polypeptide exhibits a biological activity. For example, an olfactory receptor is functionally expressed by a cell when said receptor, following its production, is transported and incorporated into the cellular membrane and is able to trigger its corresponding signalling cascade following its activation by a ligand. Conventional methods assessing the functional expression of an olfactory receptor involve the expression of said OR with the co-expression of a luciferase gene operably linked to a cAMP-inducible promoter (Saito et al. (2004) Cell 119 (5): 679-691), which is used as a reporter gene. If the olfactory receptor is functionally expressed, its activation and subsequent resulting increase in intracellular cAMP results in expression of luciferase. Cleavage of luciferin by luciferase in standard assays results in the emission of light which can then be detected and quantified. Similar approaches can be utilized for assessing the functional expression of an OR accessory molecule. A definition of “accessory molecules” is provided later herein.

The present invention, as described later herein and demonstrated in the experimental section, provides for improved methods for selecting or screening for functional expression of olfactory receptors and/or accessory molecules.

Functional expression of a polypeptide such as an olfactory receptor or an accessory molecule may be improved (increased) relative to a baseline functional expression, leading to improved (increased) biological activity. Said improved (increased) functional expression may, for example, arise from the occurrence of genetic and/or epigenetic modifications in an OR-expressing cell, relative to a non-modified corresponding cell. Said functional expression may be improved (increased) by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a non-modified corresponding cell. Improvement of functional expression may also be such that functional expression of olfactory receptors otherwise not possible using conventional approaches is achieved using the nucleic acid constructs, cells, and methods of the invention.

As used herein, a “nucleic acid construct” refers to a DNA molecule comprising a region (coding region or ORF), which is transcribed into an RNA molecule (e.g. an mRNA molecule) in a cell, operably linked to a suitable regulatory region such as, but not limited to, a promoter and/or enhancer sequence. A nucleic acid construct will generally comprise multiple operably linked fragments, such as a promoter, an enhancer, a 5′ leader sequence, a coding region, and/or a 3′ untranslated region (3′-end) e.g. comprising a polyadenylation and/or transcription termination site. A nucleic acid construct may be recombinant, i.e. not normally found in nature, such as a nucleic acid construct wherein the promoter is not associated in nature with part or all of the coding region. Molecular toolbox techniques for preparation of nucleic acid constructs are well-known in the art and are discussed in standard handbooks such as Ausubel et al., Current Protocols in Molecular Biology, 3rd edition (2003), John Wiley & Sons Inc and Sambrook and Green, Molecular Cloning: A Laboratory Manual, 4th Edition (2012), Cold Spring Harbor Laboratory Press; both of which are incorporated herein by reference in their entireties. Non-limiting examples of such techniques, some of which are demonstrated in the experimental section herein, are fusion PCR, restriction digestion, Golden-gate cloning, and the like.

The term nucleic acid construct also encompasses expression vectors. An “expression vector”, alternatively referred to herein as “vector” or “delivery vector”, refers to a molecular biology tool used to obtain expression of a coding region (such as a gene) in a host cell, for example by introducing a nucleotide sequence that is capable of effecting expression of a gene or a coding sequence in a host cell compatible with said sequence. An expression vector may be able to stabilize and remain episomal in a host cell. Alternatively, a vector may be able to integrate into a host cell's genome, for example through homologous recombination, non-homologous end-joining, or otherwise. A definition of a “host cell” is provided elsewhere herein.

Suitable expression vectors may be selected from any genetic element known in the art which can facilitate transfer of nucleic acids between cells, such as, but not limited to, plasmids, phages, transposons, cosmids, chromosomes, artificial chromosomes, viruses (such as, but not limited to, retroviruses, lentiviruses, and the like), virions, and the like. An expression vector may also be a chemical vector, such as a lipid complex or naked DNA. “Naked DNA” or “naked nucleic acid” refers to a nucleic acid molecule that is not contained in encapsulating means that facilitates delivery of a nucleic acid into the cytoplasm of a target host cell. Naked DNA may be circular or linear (linearized DNA sequence). Optionally, a naked nucleic acid can be associated with standard means used in the art for facilitating its delivery of the nucleic acid to the target host cell, for example to facilitate the transport of the nucleic acid through the cell membrane. A preferred expression vector is a plasmid. Suitable plasmids are known in the art and described in standard handbooks such as Ausubel et al. and Sambrook and Green (supra). Suitable plasmids may also be selected from commercially available vectors, such as the pcDNA3.1(+) series (Invitrogen, MA, USA) or the pGL4.29 series of vectors (Promega, WI, USA).

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are generally contiguous and, where necessary to join two protein encoding regions, contiguous and in reading frame. Linking can be accomplished by ligation at convenient restriction sites or at adapters or linkers inserted in lieu thereof, or by gene synthesis.

A nucleic acid construct according to the invention comprises a promoter and/or enhancer sequence operably linked to a nucleic acid sequence encoding a polypeptide. As used herein, the term “promoter” or “transcription regulatory sequence” refers to a nucleic acid sequence that functions to control the transcription of one or more coding sequences (i.e. expression), is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter, such as a cAMP responsive element (CRE), a half CRE, or an NFAT responsive element (NFAT-RE) as described later herein.

As used herein, the term “enhancer” refers to a nucleic acid sequence that can stimulate the transcription of a sequence it is operably linked to. An operably linked enhancer does not necessarily need to be contiguous with a coding sequence whose transcription it controls. An enhancer may be used as single sequence or may be comprised in a fusion nucleotide sequence with other enhancers and/or a promoter as described herein.

Promoters and enhancers as described herein may be modified as compared to the corresponding naturally-occurring sequences. Such modified promoters and enhancers may be alternatively referred to herein as “derivatives” of their naturally-occurring (wild-type) versions. Suitable non-limiting modifications may be selected from nucleotide insertions, deletions, mutations and/or substitutions. Suitable modifications also encompass promoter sequence fusions with other nucleotide sequences, such as but not limited to enhancer and/or other promoter sequences. Promoter-enhancer fusions may be particularly suitable. Modification of a nucleotide sequence, i.e. genetic modification, may be performed using any recombinant DNA technique as known in the art, such as for example described in standard handbooks like Ausubel et al. and Sambrook and Green (supra).

A promoter and/or enhancer as described herein may be inducible by an olfactory receptor. The term “inducible promoter” as used herein refers to a promoter or derivative thereof that only initiates transcription upon contact with a physiological or chemical inducer. The skilled person understands that inducible promoters or derivatives thereof may still allow for detectable levels of transcription of a coding sequence in the absence of the inducer (“leaky” expression). Leaky expression may mean that the inducible promoter or derivative thereof allows for at least one-fold, at least two-fold, at least three-fold, at least four-fold, at least five-fold, at least ten-fold, or at least a hundred-fold lower level of transcription of a coding sequence in the absence of an inducer relative to the presence of said inducer. Expression may be evaluated on the level of mRNA or protein by standard assays known to the person of skill in the art (e.g. qPCR, Western blotting, ELISA).

An olfactory receptor may induce a promoter and/or enhancer as described herein via inducer molecules that are produced upon activation of said receptor by a ligand. Said inducer molecules may induce a promoter and/or enhancer directly (i.e. by directly binding to the respective nucleic acid), or indirectly by triggering protein signalling cascades resulting in said promoter and/or enhancer activation. Inducer molecules associated with OR activation may be selected from signalling molecules such as inositol triphosphate (IP3), cyclic adenosine monophosphate (cAMP), cyclic guanosine monophosphate (cGMP), Ca²⁺, and the like, preferably the inducer molecule is cAMP or Ca²⁺.

A preferred promoter and/or enhancer according to the invention comprises one or more copies of a cAMP responsive element (CRE), a half CRE, or an NFAT responsive element (NFAT-RE). In some embodiments, a promoter and/or enhancer comprises two or more copies of a cAMP responsive element (CRE), a half CRE, or an NFAT responsive element (NFAT-RE). In some embodiments, a promoter and/or enhancer comprises three of more copies of a cAMP responsive element (CRE), a half CRE, or an NFAT responsive element (NFAT-RE). In some embodiments, a promoter and/or enhancer comprises four or more copies of a cAMP responsive element (CRE), a half CRE, or an NFAT responsive element (NFAT-RE).

In some embodiments, a promoter and/or enhancer comprises a combination of one or more copies of a cAMP responsive element (CRE) and one or more copies of a half CRE.

In some embodiments, a promoter and/or enhancer comprises a combination of one or more copies of a cAMP responsive element (CRE) and one or more copies of an NFAT responsive element (NFAT-RE).

In some embodiments, a promoter and/or enhancer comprises a combination of one or more copies of a half-CRE and one or more copies of an NFAT responsive element (NFAT-RE).

In some embodiments, a promoter and/or enhancer comprises a combination of one or more copies of a cAMP responsive element (CRE) and one or more copies of a half-CRE and one or more copies of an NFAT responsive element (NFAT-RE).

The cAMP responsive element (CRE) or the half CRE are nucleic acid sequences which, when comprised in a promoter and/or enhancer, result in said promoter and/or enhancer being inducible by cAMP. Such a promoter and/or enhancer may be called a cAMP-responsive promoter and/or enhancer. The mechanism of CRE or half CRE activation is known in the art. Typically, in cells functionally expressing olfactory receptors, the activation of a G-protein upon activation of the olfactory receptor by a ligand, said G-protein being a Gα_{olf} in the case of olfactory neurons or another G-protein such as GαS in other types of cells, and subsequent production of cAMP leads to activation of protein kinase A (PKA). Typically, activation of PKA results in the phosphorylation of the cAMP-response-element binding protein 1 (CREB), which is a 43 kDa stimulus-induced transcription factor (TF). CREB may bind to a cAMP responsive element or half CRE, resulting in the transcription of the operably linked nucleic acid sequence encoding a polypeptide.

A cAMP responsive element (CRE) may be represented by the nucleic acid sequence 5′-TGACGTCA-3′ (SEQ ID NO: 1). A half CRE may be represented by the nucleic acid sequence 5′-TGACG-3′ (SEQ ID NO: 2). Accordingly, in some embodiments, a promoter and/or enhancer comprises one or more copies of SEQ ID NO: 1. In some embodiments, a promoter and/or enhancer comprises two or more copies of SEQ ID NO: 1. In some embodiments, a promoter and/or enhancer comprises three or more copies of SEQ ID NO: 1. In some embodiments, a promoter and/or enhancer comprises four or more copies of SEQ ID NO: 1. In some embodiments, a preferred promoter and/or enhancer comprises one or more copies of SEQ ID NO: 2. In some embodiments, a promoter and/or enhancer comprises two or more copies of SEQ ID NO: 2. In some embodiments, a promoter and/or enhancer comprises three or more copies of SEQ ID NO: 2. In some embodiments, a promoter and/or enhancer comprises four or more copies of SEQ ID NO: 2.

A promoter and/or enhancer according to the invention may comprise a combination of one or more copies of a cAMP responsive element (CRE) and one or more copies of a half CRE. Exemplary nucleic acid sequences are represented by SEQ ID NOs: 3 and 4. Accordingly, in some embodiments, a preferred promoter and/or enhancer comprises, consists essentially of, or consists of, one or more copies of a nucleic acid sequence represented by SEQ ID NO: 3 or SEQ ID NO: 4, preferably SEQ ID NO: 4, or one or more copies of a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 3 or SEQ ID NO: 4, preferably SEQ ID NO: 4.

The mechanism of NFAT responsive element activation is known in the art. The NFAT-RE (nuclear factor of activated T-cells response element) is a nucleic acid sequence which, when comprised in a promoter and/or enhancer, typically results in said promoter and/or enhancer being indirectly inducible by Ca²⁺. Typically, in olfactory neuron cells functionally expressing olfactory receptors, the signalling cascade upon activation of the olfactory receptor by a ligand, results in the entry of Ca²⁺ into the OR-expressing cell. In cells with exogenous functional expression of olfactory receptors, entry of Ca²⁺ into the cytosol may be triggered by introduction of nucleotide sequences encoding cyclic nucleotide-gated ion channels (CNG channels), which allow calcium to enter the cells upon formation of cAMP. A definition of “exogenous” expression is provided later herein. Non-limiting examples of a cyclic nucleotide-gated ion channel are CNGA1 (NCBI Genbank Gene ID: 1259), CNGA2 (NCBI Genbank Gene ID: 1260), CNGA3 (NCBI Genbank Gene ID: 1261), CNGA4 (NCBI Genbank Gene ID: 1262), CNGB1 (NCBI Genbank Gene ID: 1258), and CNGB3 (NCBI Genbank Gene ID: 54714). Alternatively, chimeric G-proteins may be used, described in Conklin et al. (1993) Nature 363:274-276, which is incorporated herein by reference in its entirety, which can activate phospholipase C (PLC) in presence of cAMP, resulting in IP3 production that results in release of Ca²⁺ from internal stores. In said proteins, the three C-terminal amino acids of G_{q}α are replaced with the corresponding residues of Ga. The increase in intracellular Ca²⁺ results in activation of calmodulin, which in turn results in activation of nuclear factors of activated T-cells (NFATs). NFATs are a transcription factor (TF) family comprising NFATc1, NFATc2, NFATc3, NFATc4, and NFAT5. NFATc1 through NFATc4 are regulated by calcium signalling, and are known as the classical members of the NFAT family. The activation of NFATs results in the activation of an NFAT-RE, resulting in the transcription of the operably linked nucleic acid sequence encoding a polypeptide. Accordingly, a promoter and/or enhancer comprising one or more copies of an NFAT responsive element (NFAT-RE) may be called an NFAT-responsive promoter and/or enhancer. The skilled person understands that a promoter and/or enhancer according to the invention may also simultaneously be cAMP-responsive and NFAT-responsive.

An NFAT-RE typically comprises one or more NFAT-binding sites which may be represented by the nucleic acid sequence 5′-GGAAAA-3′ (SEQ ID NO: 5). Accordingly, in some embodiments, a promoter and/or enhancer comprises one or more copies of SEQ ID NO: 5. In some embodiments, a promoter and/or enhancer comprises two or more copies of SEQ ID NO: 5. In some embodiments, a promoter and/or enhancer comprises three or more copies of SEQ ID NO: 5. In some embodiments, a promoter and/or enhancer comprises four or more copies of SEQ ID NO: 5.

An NFAT-responsive promoter and/or enhancer typically additionally comprises one or more binding sites for the transcription factor AP-1 (activator protein 1). An exemplary sequence is represented by SEQ ID NO: 6. Accordingly, in some embodiments, a preferred promoter and/or enhancer comprises, consists essentially of, or consists of, one or more copies of a nucleic acid sequence represented by SEQ ID NO: 6, or one or more copies of a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 6.

A nucleic acid construct according to the invention comprises a nucleic acid sequence encoding a polypeptide. In some embodiments, said nucleic acid sequence is a selectable marker, i.e. is a sequence that encodes a polypeptide that can be used for selection of host cells expressing said nucleic acid sequence by conferring a selective advantage to said cells upon exposure to selective conditions. A selectable marker may enable positive or negative selection. Suitable selection markers are known in the art and such markers and selection methods are discussed e.g. in standard publications such as Mortensen and Kingston (2009) Curr Protoc Mol Biol 86:9.5.1-9.5.13, incorporated herein by reference in its entirety. The skilled person understands that the application of a specific selectable marker may enable positive or negative selection depending on the host cell and/or the selective conditions which are applied. Positive selectable markers are markers that enable survival and/or growth of the host cell upon exposure to selective conditions wherein survival and/or growth would otherwise not occur. Non-limiting examples of such markers are markers that confer resistance to a toxic compound such as an antibiotic, markers that enable utilization of unusual carbon and/or nitrogen sources, markers that complement carbon, nitrogen, and/or micronutrient auxotrophies which arise from mutations or the application of selective conditions (such as, but not limited to cytosine deaminase (EC 3.5.4.1), dihydrofolate reductase (EC. 1.5.1.3), histidinol dehydrogenase (EC 1.1.1.23), thymidine kinase (2.7.1.21), or xanthine-guanine phosphoribosyltransferase (EC 2.4.2.8) and the like. A preferred selectable marker is a nucleic acid sequence encoding a polypeptide which confers resistance to an antibiotic. Non-limiting examples of such nucleic acid sequences are the puromycin-N-acetyltransferase gene (pac, e.g. as represented by SEQ ID NO: 7) which confers resistance to puromycin, the hygromycin-B-phosphotransferase gene (hph, e.g. as represented by SEQ ID NO: 8) which confers resistance to hygromycin B (hygrovetine), the aminoglycoside 3′-phosphotransferase gene (neo, e.g. from Klebsiella pneumoniae, UniprotKB Ref: NODR31) which confers resistance to geneticin (G418), the Sh ble gene (e.g. from Streptoalloteichus hindustanus, UniprotKB Ref: P17493) which confers resistance to zeocin and other antibiotics of the bleomycin family, and the blasticidin-S deaminase gene (bsd, e.g. from Aspergillus terreus, UniprotKB Ref: POC2P0) which confers resistance to blasticidin (also known as blasticidin S). Another example of a blasticidin-S deaminase is represented by the amino acid sequence of SEQ ID NO: 65 and/or encoded by a nucleotide sequence represented by SEQ ID NO: 64. In some embodiments wherein the selectable marker encodes a polypeptide which confers resistance to an antibiotic, a puromycin-N-acetyltransferase gene is preferred. Accordingly, in some embodiments, the polypeptide which confers resistance to an antibiotic is a puromycin-N-acetyltransferase. Another preferred selectable marker that encodes a polypeptide which confers resistance to an antibiotic is a blasticidin-S deaminase gene. Accordingly, in some embodiments, the polypeptide which confers resistance to an antibiotic is a blasticidin-S deaminase.

The use of blasticidin-S deaminase as a selectable marker, and the associated conferred resistance to blasticidin, may be particularly advantageous in conjunction with the methods described herein. As shown in the experimental section herein, and compared to selection with other antibiotics, selection with blasticidin may offer an enhanced dynamic range, i.e., a large difference in measured viability between cells functionally expressing an OR in the presence of the ligand and cells that survive without functional OR expression or in the absence of the ligand. Cell viability and its measurement is further discussed later herein.

In some embodiments, a nucleic acid construct comprises, consists essentially of, or consists of, a nucleic acid sequence represented by SEQ ID NO: 7 or 8, or a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 7 or 8. In some embodiments, a nucleic acid construct comprises, consists essentially of, or consists of, a nucleic acid sequence represented by SEQ ID NO: 7, 8, or 64, preferably SEQ ID NO: 64, or a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 7, 8, or 64, preferably SEQ ID NO: 64.

Negative selectable markers are markers that eliminate or inhibit growth upon exposure to selective conditions wherein growth would otherwise occur. Non-limiting example of such markers are Herpes simplex thymidine kinase (UniprotKB Ref: Q9QNF7) which renders the host cell sensitive to ganciclovir selection and cytosine deaminase (EC 3.5.4.1) which renders the host cell sensitive to 5-fluorocytosine selection.

In some embodiments, a nucleic acid construct according to the invention comprises a nucleic acid sequence encoding a polypeptide, wherein said polypeptide is a reporter polypeptide. The expression of said polypeptide is indicative of the activation of the olfactory receptor. Said expression may be directly or indirectly detectable. A “reporter polypeptide” is a polypeptide the expression of which results in a measurable signal. “Direct detection” refers to the measurable signal being directly measurable. A non-limiting example of a directly detectable reporter polypeptide is any variant of a fluorescent protein such as green fluorescent protein (GFP) or any variant with a different fluorescence spectrum such as red fluorescent protein. Such polypeptides are known to the skilled person and discussed in standard handbooks, such as Chalfie and Kain, Green Fluorescent Protein: Properties, Applications and Protocols (Methods of Biochemical Analysis), 2nd Edition (2005), Wiley-Liss (incorporated herein by reference in its entirety), and in publicly available databases such as FPbase, described in Lambert (2019) Nature Methods 16:277-278, incorporated herein by reference in its entirety (www.fpbase.org). Expression of such a polypeptide following the activation of an olfactory receptor results in the emittance of a fluorescent signal, which can be detected and utilized for enriching cells using commercially available devices such as a fluorescence-activated cell sorting device (FACS) or a device able to automatically pick fluorescent clonal colonies such as the ClonePix2 colony picker (Molecular Devices, CA, USA). Alternatively, any enzyme known to the skilled person which can form fluorescent products which are retained within the cells, thereby enabling the detection of a fluorescent signal by a commercial device as discussed above may be used. Accordingly, in some embodiments, the encoded polypeptide is detectable with a fluorescence-activated cell sorting device.

“Indirect detection” will typically require an additional step before a measureable signal resulting from reporter polypeptide expression is obtained. A non-limiting example of an indirectly detectable reporter polypeptide is an antigen which is recognizable by a fluorescent-labelled antibody. Expression of such a polypeptide following the activation of the olfactory receptor followed by incubation with such an antibody using standard protocols will allow for fluorescent signal detection using commercially available devices as described above. Alternatively, reporter polypeptides such as, but not limited to, β-galactosidase (EC 3.2.1.23), β-lactamase (EC 3.5.2.6), catalase (EC 1.11.1.6), and the like, may be used, said polypeptides catalyzing reactions that result in formation of detectable colored products when brought into contact with their respective substrates in standardly used assays, such as for example described in Kasper et al. (2016) Methods Mol Biol 1453:123-36, incorporated herein by reference in its entirety. Expression of reporter polypeptides may be combined with commercially available substrates, such as CellEvent™ (ThermoFisher Scientific, MA, USA) to facilitate detection.

The functional relationship between a promoter and/or enhancer inducible by an olfactory receptor with a nucleic acid sequence encoding a polypeptide such as a selectable marker or a reporter polypeptide as described herein may be experimentally confirmed without olfactory receptor activation using standard methods in the art, for example as described in standard publications like Alasbahi and Melzig, (2012) Pharmazie 67 (1): 5-13, incorporated herein by reference in its entirety. As a non-limiting example, a selectable marker conferring resistance to an antibiotic operably linked to a promoter and/or enhancer comprising one or more copies of a cAMP responsive element (CRE) and/or a half CRE may be introduced to the genome of a host cell. Said cells may then be cultured in the presence of the cAMP-inducing agent forskolin and the antibiotic according to standard methods and conditions. Forskolin-induced resistance to the antibiotic (caused by an increase in intracellular cAMP such as arising from the activation of an olfactory receptor) demonstrates the functional relationship.

In the context of the invention, an olfactory receptor may be expressed by a host cell. An olfactory receptor may be a variant (alternatively referred to herein as mutant), i.e. an olfactory receptor that is modified as compared to the corresponding naturally-occurring sequence. Preferably, said expression is functional expression. Expression of an olfactory receptor may be endogenous or exogenous. Endogenous expression refers to expression of an olfactory receptor by a cell that is natively able to express it, i.e. a cell that comprises the required genetic information for its expression, e.g. an olfactory sensory neuron cell. Exogenous expression typically refers to expression of an olfactory receptor by a different organism and/or cell, in which the olfactory receptor is not natively expressed, the capability of which having been introduced via means of recombinant DNA technology. Within the context of the invention, the term exogenous expression also encompasses cases wherein the native expression of an olfactory receptor is increased via means of recombinant DNA technology using standard molecular toolbox techniques (e.g. overexpression) relative to the corresponding native expression. Said increase may be achieved by modification of any of the olfactory receptor expression steps, including transcription, post-transcriptional modification, translation, post-translational modification and transport to the cellular membrane. Said increase may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200% relative to the corresponding native expression. Expression may be evaluated on the level of mRNA or protein by standard assays known to the person of skill in the art (e.g. qPCR, Western blotting, ELISA).

Accordingly, the invention further provides a nucleic acid construct as defined earlier herein, comprising a nucleic acid sequence encoding an olfactory receptor. The nucleic acid sequence encoding an olfactory receptor may be operably linked to a promoter and/or enhancer. Said promoter may be constitutive, i.e. allowing for constant expression, such as, but not limited to the CMV promoter (SEQ ID NO: 15). Said promoter may be inducible. The nucleic acid construct comprising a nucleic acid molecule encoding an olfactory receptor may be a separate nucleic acid construct or may be fused with the nucleic acid construct comprising a nucleic acid sequence comprising a promoter and/or enhancer sequence operably linked to a nucleic acid sequence encoding a polypeptide, wherein said promoter and/or enhancer is inducible by an olfactory receptor as described earlier herein, so as to constitute a single nucleic acid construct, preferably the two constructs are fused. Non-limiting examples of nucleic acid sequences encoding an olfactory receptor are OR10G9 (NCBI Genbank Gene ID: 219870), OR5AN1 (NCBI Genbank Gene ID: 390195, SEQ ID NO: 70), OR5A2 (NCBI Genbank ID: 219981), OR5A1 (NCBI Genbank Gene ID: 219982), OR6Y1 (NCBI Genbank Gene ID: 391112), OR10G4 (NCBI Genbank Gene ID: 390264, SEQ ID NO: 72), and OR10G7 (NCBI Genbank Gene ID: 390265, SEQ ID NO: 71). Additional examples of nucleic acid sequences encoding an olfactory receptor may be found in publications such as Mainland et al. (supra), and in publicly available databases, such as the HORDE (The Human Olfactory Data Explorer) database (supra).

In some embodiments, a nucleic acid construct, preferably a plasmid, comprises a nucleic acid sequence encoding an olfactory receptor and a promoter and/or enhancer sequence operably linked to a nucleic acid sequence encoding a polypeptide, preferably a selectable marker, more preferably a selectable marker conferring resistance to an antibiotic, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE). Preferably, the selectable marker is a puromycin-N-acetyltransferase gene or a blasticidin-S deaminase gene, more preferably a blasticidin-S deaminase gene.

Optionally, additional nucleic acid sequences may be operably linked to the nucleotide sequences comprised in any of the nucleic acid constructs described herein. Non-limiting examples of such sequences include nucleic acid sequences encoding signal peptides such as N-terminal LUCY-tags (SEQ ID NO: 9, SEQ ID NO: 10), FLAG-tags (SEQ ID NO: 11), and rho-tags (SEQ ID NO: 12), such as described in Shepard et al. (2013) PLOS One 8 (7): e68758, in Zhuang and Matsunami (2007) J Biol Chem 282 (20): 15284-15293, and in WO2014/037800, each of which is incorporated herein by reference in its entirety. A further example of a nucleic acid sequence encoding a signal peptide is represented by SEQ ID NO: 13. Additional non-limiting examples of nucleic acid sequences include nuclear localization signals, kozak sequences, polyA-tails, transcription terminators such as the bovine growth hormone (bgh) terminator sequence (SEQ ID NO: 16), and the like.

In some embodiments, a nucleic acid construct comprises, consists essentially of, or consists of, a nucleic acid sequence encoding a polypeptide represented by SEQ ID NOs: 9, 10, 11, 12, or 14, preferably SEQ ID NO: 14, or a nucleotide sequence encoding a polypeptide having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NOs: 9, 10, 11, 12, or 14, preferably SEQ ID NO: 14.

In some embodiments, a nucleic acid construct comprises, consists essentially of, or consists of, a nucleic acid sequence represented by SEQ ID NO: 13, or a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 13.

Optionally, additional nucleic acid sequences encoding accessory molecules or other proteins may be comprised in any of the nucleic acid constructs described herein. “Accessory molecules” or “chaperones” are proteins or peptides that may assist in the expression, trafficking, and/or signalling of an olfactory receptor to the surface of a cell expressing said olfactory receptor. An accessory molecule may be a variant (alternatively referred to herein as mutant), i.e. an accessory molecule that is modified as compared to the corresponding naturally-occurring sequence. Non-limiting examples of accessory molecules or other proteins include RTPL1, RTP1S, RTP2, REEP, the RTP1S V2271 variant, the RTP2 L220R variant, β-adrenergic receptor, heat shock protein 70, Ric8b, Gα_{olf}, Giα, or variants thereof, and the like, and are further described in WO2006/002161 and WO2014/037800, incorporated herein by reference in their entireties. Preferred accessory molecules are the human RTP1S V2271 variant (SEQ ID NO: 17) and the human RTP2 L220R variant (SEQ ID NO: 18). In some embodiments, a nucleic acid construct comprises, consists essentially of, or consists of, a nucleic acid sequence encoding a polypeptide represented by SEQ ID NO: 17 or 18, or a nucleotide sequence encoding a polypeptide having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 17 or 18.

Additional nucleic acid sequences that may be comprised in a nucleic acid construct described herein are additional selectable markers, for example selectable markers conferring resistance to an antibiotic, that are constitutively expressed. Such markers may, for example, be used in selecting host cells that comprise the nucleic acid construct of the invention prior to the application of the methods described herein. A non-limiting example of such a marker is the hygromycin-B-phosphotransferase gene (hph, e.g. as represented by SEQ ID NO: 8) which confers resistance to hygromycin B (hygrovetine).

In some embodiments, a nucleic acid construct, preferably a plasmid, comprises a nucleic acid sequence encoding an olfactory receptor, a promoter and/or enhancer sequence comprising one or multiple copies of an NFAT responsive element (NFAT-RE) operably linked to a nucleic acid sequence encoding a polypeptide, preferably a selectable marker, more preferably a selectable marker conferring resistance to an antibiotic, and a nucleic acid sequence encoding a cyclic nucleotide-gated ion channel, as described earlier herein. Preferably, the selectable marker is a puromycin-N-acetyltransferase gene or a blasticidin-S deaminase gene, more preferably a blasticidin-S deaminase gene.

In some embodiments, a nucleic acid construct, preferably a plasmid, comprises a nucleic acid sequence encoding an olfactory receptor, a promoter and/or enhancer sequence comprising one or multiple copies of an NFAT responsive element (NFAT-RE) operably linked to a nucleic acid sequence encoding a polypeptide, preferably a selectable marker, more preferably a selectable marker conferring resistance to an antibiotic, and a nucleic acid sequence encoding a chimeric G-protein which is able to activate phospholipase C, as described earlier herein. Preferably, the selectable marker is a puromycin-N-acetyltransferase gene or a blasticidin-S deaminase gene, more preferably a blasticidin-S deaminase gene.

Coding sequences and genes as described herein may be codon optimized for expression in a host cell, preferably in a eukaryotic cell, more preferably in a human cell. “Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host cell or organism. Codon optimization also eliminates elements that potentially impact negatively RNA stability and/or translation (e. g. termination sequences, TATA boxes, splice sites, ribosomal entry sites, repetitive and/or GC rich sequences and RNA secondary structures or instability motifs). In some embodiments, codon-optimized sequences show at least 3%, 5%, 10%, 15%, 20%, 25%, 30%, 40%, 50%, 60%, 70%, 80%, 90%, 100% or more increase in gene expression, transcription, RNA stability and/or translation compared to the original, non-codon-optimized sequence.

### Host Cell

The nucleic acid constructs described herein are particularly useful for introduction into a host cell. Accordingly, in a second aspect, the invention provides a host cell comprising a nucleic acid construct as defined earlier herein.

In some embodiments, the host cell comprises a nucleic acid construct comprising a promoter and/or enhancer sequence operably linked to a nucleic acid sequence encoding a polypeptide, preferably a selectable marker, more preferably a selectable marker conferring resistance to an antibiotic, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor. Preferably, said nucleic acid constructs are fused so as to constitute a single nucleic acid construct, more preferably a single plasmid. In some embodiments, a preferred selectable marker is a puromycin-N-acetyltransferase gene or a blasticidin-S deaminase gene, more preferably a blasticidin-S deaminase gene.

A “host cell”, alternatively referred to herein as “cell” or “engineered cell”, refers to a cell that has been engineered by the introduction of a nucleic acid construct as defined herein. A host cell may refer to a cell in isolation or in culture. Host cells may be “transduced cells”, wherein the cells have been infected with e.g. a modified virus. As a non-limiting example a lentivirus may be used, but other suitable viruses such as retroviruses or others may be contemplated as well. Introduction of a nucleic acid construct may also be performed by non-viral methods, e.g. by transfection. “Transfection” refers to non-viral methods of DNA (or RNA) transfer to cells such that the transferred nucleic acid sequence is expressed. Transfection methods and protocols are well-known in the art, with non-limiting examples being calcium phosphate transfection, PEG transfection, and liposomal or lipoplex transfection, and discussed in standard handbooks such as Ausubel et al. and Sambrook and Green (supra). A further example of a transfection method is provided in the exemplary section herein. A transfection may be transient or stable, the latter referring to cases wherein cells have the nucleic acid construct integrated in their genome. Host cells comprising a nucleic acid construct as described herein may thus also be “stably transfected cells” or “transiently transfected cells”.

A host cell may be further genetically modified, for example by the introduction of one or more genetic modifications including, but not limited to, nucleotide mutations, substitutions, insertions, and/or deletions in its genome, and/or introduction of additional nucleic acid constructs. Said modifications may be comprised in a nucleotide sequence encoding an olfactory receptor, an accessory molecule, and/or another genomic region and may result in functional expression or improved functional expression of said olfactory receptor and/or said accessory molecule. A definition of functional expression is provided earlier herein.

Modification of a nucleic acid sequence may be performed using any recombinant DNA technique as known in the art, such as for example described in standard handbooks such as Ausubel et al. and Sambrook and Green (supra). Also see, Kunkel (1985) Proc. Natl. Acad. Sci. 82:488 (describing site directed mutagenesis) and Roberts et al. (1987) Nature 328:731 734 or Wells, J. A., et al. (1985) Gene 34:315 (describing cassette mutagenesis).

Alternatively, further genetic modifications may be introduced by mutagenesis techniques known in the art such as cell irradiation with ultraviolet light or chemical mutagenesis by exposure of cells to known mutagens such as, but not limited to, alkylating agents such as N-ethyl-N-nitrosourea or ethyl methanesulfonate. Alternatively, further genetic modifications may be introduced by insertional mutagenesis of a nucleic acid sequence, i.e. targeted or random insertion of DNA sequences into the host cell's genome, such as in the vicinity or inside gene sequences, mediated by, for example, but not limited to, plasmids, linearized DNA sequences, transposons, lentiviruses, retroviruses, or CRISPR-Cas-mediated recombination (i.e. insertion of nucleic acid sequences into the genome, following a double stranded DNA break induced by CRISPR-Cas, via cellular DNA repair machinery such as homologous recombination or non-homologous end-joining). An inserted nucleic acid sequence may comprise a splice acceptor site and/or a polyadenylation signal. Said sequence may be able to block gene expression at the insertion site by causing incorrect splicing and/or early transcription termination (loss-of-function mutations). An inserted nucleic acid sequence may comprise an enhancer and/or promoter sequence. Said sequence may optionally further (or instead) comprise a splice donor site. Said sequence may be able to activate expression of endogenous genes. Said activation may arise from the promoting of gene expression at the insertion site by promoting transcription and/or correct splicing (gain-of-function mutations).

Insertional mutagenesis methods and protocols using plasmids, linearized DNA sequences, transposons, lentiviruses, retroviruses, and CRISPR-Cas-mediated recombination are well-known in the art and described in standard publications such as Kandel et al. (2005) PNAS 102:6425-30, Montini et al. (2009) J Clin Invest 119:964-75, Ranzani et al. (2013) Curr Protoc Mol Biol Chapter 9: Unit9.5, Ranzani et al. (2014) Mol Ther 22 (12): 2056-2068, Feddersen et al. (2019) BMC Genomics 20:497, and Yang et al. (2014) In: Storici F. (eds) Gene Correction. Methods in Molecular Biology (Methods and Protocols), 1114. Humana Press, NJ, USA, each of which incorporated herein by reference in its entirety.

Alternatively, further genetic modifications may be introduced by CRISPR-Cas-mediated mutagenesis, which may be used for loss-of-function or gain-of-function mutagenesis, using CRISPR interference (CRISPRi) or CRISPR activation (CRISPRa), respectively. Said methods rely on engineered versions of deactivated Cas9 (dCas9) which are able to modulate gene expression when complexed with a repressor protein in CRISPRi (e.g. dCas9-KRAB), which may lead to loss-of-function mutagenesis, or activator protein in CRISPRa (e.g. dCas9-VPH), which may lead to gain-of-function mutagenesis, and subsequently targeted to promoter regions of gene targets using specifically synthesized guide RNAs (sgRNAs). Guide RNAs, dCas9 proteins, and protocols for gene targeting to apply CRISPRi and CRISPRa are well-known in the art, with many being commercially available, e.g. the CRISPRa/CRISPRi dCas9 and sgRNA libraries and protocols supplied by Cellecta Inc. (CA, USA).

Furthermore, genetic modifications may be introduced by evolutionary engineering of host cells. Evolutionary engineering typically relies on, (i) optionally generating host cell genetic diversity by introducing genetic modifications using any recombinant DNA technique known in the art, for example mutagenesis techniques such as described above, and (ii) linkage of a selectable marker and/or trait with the phenotype of interest, followed by the application of selective pressure on the host cells. When said host cells are sub-cultured over multiple generations, cells having acquired mutations leading to improved phenotypes will have a selective advantage and will be enriched in the culture. Evolutionary engineering may result on gain-of-function and/or loss-of-function mutations, depending on the selectable marker and/or trait and the applied selective pressure. As a non-limiting example, a nucleic acid construct comprising a nucleic acid sequence encoding an antibiotic resistance gene, e.g., a puromycin-N-acetyltransferase gene or a blasticidin-S deaminase gene, operably linked to a promoter and/or enhancer inducible by an olfactory receptor and a nucleic acid sequence encoding an olfactory receptor may be introduced in a host cell. Upon exposure to the ligand of the olfactory receptor and the respective antibiotic in a culture, only host cells which are able to functionally express the olfactory receptor will induce the expression of the antibiotic resistance and will be able to survive and/or grow. When multiple sub-culturing steps are applied, host cells that have acquired mutations that enable them to more efficiently express said olfactory receptor will be enriched in the culture and may then be isolated/selected. A further example of such an approach is given in the exemplary section herein.

A host cell may comprise epigenetic modifications in a nucleic acid molecule encoding an olfactory receptor, an accessory molecule, and/or another genomic region which may result in functional expression or improved functional expression of said olfactory receptor and/or said accessory molecule. As used herein, the term “epigenetic modification” has its customary meaning as ordinarily understood by the skilled person in view of this disclosure. It refers to chemical modifications of DNA or histone proteins that do not alter a nucleotide sequence itself. Non-limiting examples of epigenetic modifications include nucleic acid methylation, acetylation, phosphorylation, serotonylation, citrullination, ubiquitination, sumoylation, and ribosylation.

Additional nucleic acid constructs that may be comprised in a host cell may comprise nucleic acid sequences encoding accessory molecules or other proteins as described earlier herein. Preferred such constructs comprise nucleic acid sequences encoding for the V2271 variant of the human RTP1S and/or L220R variant of the human RTP2 (SEQ ID NO: 17 and/or 18), as described earlier herein.

Host cells may be prokaryotic or eukaryotic cells, preferably they are eukaryotic cells. Suitable prokaryotic cells may be selected from bacteria and archaea. Suitable eukaryotic cells may be selected from insect, plant, yeast, fungal, algal, mammalian, and human cells, of which human cells are preferred.

Suitable host cells include, but are not limited to, HEK293, HEK293T, HeLa, CHO, OP6, HeLa-S3, HEKn, HEKa, PC-3, Calul, Hep G2, HeLa B, HeLa T4, COS, COS-1, COS-6, C0S-M6A, BS-C-1 monkey kidney epithelial cells, BALB/3T3 mouse embryo fibroblasts, 3T3 Swiss, 3T3-L1, 132-d5 human fetal fibroblasts, 10.1 mouse fibroblasts, 293-T, 3T3, BHK, BHK-21, BR 293, BxPC3, C3H-10T1/2, C6/36, Cal-27, CHO-7, CHO-IR, CHO-K1, CHO-K2, CHO-T, CHO Dhfr−/−COS-7, HL-60, LNCap, MCF-7, MCF-IOA, MDCK II, SkBr3, Vero cells, primary olfactory cells, immortalized olfactory cells, immortalized taste cells, and transgenic varieties thereof, of which HEK293T are preferred. Cell lines are available from a variety of publicly available culture collections, e.g. the American Type Culture Collection (VA, USA).

Within the context of the invention, a host cell may be comprised in a population of cells. Accordingly, in a third aspect, the invention provides a population of cells as described herein. A population of cells may be homogeneous or heterogeneous (mixed). A “homogeneous” or “clonal” population is a population wherein all the cells comprise the same nucleic acid constructs and/or genes. A “heterogeneous” or “mixed” population, is a population wherein at least one of the cells comprises a nucleic acid construct and/or gene (e.g. a nucleic acid sequence encoding an olfactory receptor) which is distinct from the nucleic acid constructs and/or genes comprised in the other cells. Heterogeneous populations may be particularly advantageous, as they may define a pool of cells expressing distinct olfactory receptors (i.e. olfactory receptor libraries). As a non-limiting example, a library of olfactory receptors and/or accessory molecules, i.e. a pool of cells expressing distinct olfactory receptors and/or accessory molecules may be generated, allowing for high-throughput screening of novel cognate olfactory receptor-ligand pairs and/or identification of accessory molecules and/or mutations allowing for the functional expression of olfactory receptors which are otherwise typically difficult to functionally express.

In some embodiments, a population of cells comprises several cells comprising a nucleic acid construct comprising a promoter and/or enhancer sequence operably linked to a nucleic acid sequence encoding a polypeptide, preferably a selectable marker, more preferably a selectable marker conferring resistance to an antibiotic, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor, wherein the olfactory receptor encoded by the nucleic acid molecule comprised in the second nucleic acid construct comprised in at least one of the cells is distinct from the olfactory receptor encoded by the nucleic acid molecule comprised in the second nucleic acid construct in at least one of the other cells within said population, defining a pool of cells expressing distinct olfactory receptors. In some embodiments, at least one olfactory receptor is functionally expressed in said population of cells. Preferably, the selectable marker is a puromycin-N-acetyltransferase gene or a blasticidin-S deaminase gene, more preferably a blasticidin-S deaminase gene.

In some embodiments, a population of cells comprise several cells comprising a nucleic acid construct comprising a promoter and/or enhancer sequence operably linked to a nucleic acid sequence encoding a polypeptide, preferably a selectable marker, more preferably a selectable marker conferring resistance to an antibiotic, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE), and a nucleic acid molecule encoding an olfactory receptor, wherein the olfactory receptor encoded by the nucleic acid molecule in at least one of the cells is distinct from the olfactory receptor encoded by the nucleic acid molecule in at least one of the other cells within said population, defining a pool of cells expressing distinct olfactory receptors. In some embodiments, at least one olfactory receptor is functionally expressed in said population of cells. Preferably, the selectable marker is a puromycin-N-acetyltransferase gene or a blasticidin-S deaminase gene, more preferably a blasticidin-S deaminase gene.

### Methods

The present invention enables the functional expression of olfactory receptors otherwise not possible using conventional approaches. The nucleic acid constructs, cells, and populations of cells described herein are further particularly useful for use in a method for selection or screening of cells functionally expressing olfactory receptors and/or accessory molecules required for said functional expression, for identification of improved accessory molecules and/or genetic and/or epigenetic modifications required for functional or improved functional expression of olfactory receptors, and for identification of novel cognate receptor-ligand pairs. The methods of the invention are further particularly suitable for high-throughput selection or screening and cell sorting assays.

Accordingly, in a fourth aspect, the invention provides a method for selecting or screening for a cell expressing a functional olfactory receptor and/or for accessory molecules needed for said functional expression in a cell, said method comprising the following steps:
A) Providing cells as described earlier herein, wherein said cells comprise a nucleic acid construct comprising a promoter and/or enhancer sequence operably linked to a nucleic acid sequence encoding a polypeptide, wherein said encoded polypeptide confers resistance to an antibiotic or is a reporter polypeptide, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor,
B) Culturing said cells in the presence of the ligand of said olfactory receptor,
C1) Selecting for cells functionally expressing the olfactory receptor by culturing them in the presence of the antibiotic and the ligand, or
C2) Screening for cells functionally expressing the olfactory receptor by detecting and sorting out cells expressing the reporter polypeptide in the presence of the ligand.

In step A), a nucleic acid sequence encoding a polypeptide conferring resistance to an antibiotic is preferred, preferably the nucleic acid sequence is a puromycin-N-acetyltransferase gene or a blasticidin-S deaminase gene. Among steps C1) and C2), step C1) is preferred.

In a fifth aspect, the invention provides a method for selecting or screening for a cell expressing a functional olfactory receptor and/or for accessory molecules needed for said functional expression in a cell, said method comprising the following steps:
A) Applying a mutagenesis step to cells as described earlier herein, wherein said cells comprise a nucleic acid construct comprising a promoter and/or enhancer sequence operably linked to a nucleic acid sequence encoding a polypeptide, wherein said encoded polypeptide confers resistance to an antibiotic or is a reporter polypeptide, wherein said promoter and/or enhancer is inducible by an olfactory receptor, preferably wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor,
B) Culturing the mutated cells in the presence of the ligand of said olfactory receptor,
C1) Selecting for mutated cells functionally expressing the olfactory receptor by culturing them in the presence of the antibiotic and the ligand, or
C2) Screening for mutated cells functionally expressing the olfactory receptor by detecting and sorting out cells expressing the reporter polypeptide in the presence of the ligand.

In step A), a nucleic acid sequence encoding a polypeptide conferring resistance to an antibiotic is preferred, preferably the nucleic acid sequence is a puromycin-N-acetyltransferase gene or a blasticidin-S deaminase gene. Among steps C1) and C2), step C1) is preferred.

The term “selection” or “cell selection” as used herein has its customary meaning as ordinarily understood by the skilled person in view of this disclosure. It refers to the segregation and/or isolation of a cell exhibiting a phenotype of interest from a mixed population by applying selective culture conditions and/or culture media, i.e. conditions and/or media that favor the survival and/or growth of a cell exhibiting said phenotype of interest while inhibiting the survival and/or growth of all other cells. The term “screening” or “cell screening” as used herein has its customary meaning as ordinarily understood by the skilled person in view of this disclosure. It refers to the identification of a cell exhibiting a phenotype of interest by detection of measurable signal associated with said phenotype (e.g. expressing a fluorescent reporter polypeptide). The term “screening” also encompasses the post-identification sorting out of cells exhibiting the phenotype of interest, i.e. their segregation and/or isolation from a mixed population.

Unless otherwise indicated herein, the description provided for each feature of the individual steps below is applicable to both methods of the fourth and fifth aspect; the only difference being that in the method of the fifth aspect, step A) comprises the application of a mutagenesis step.

In step A) of the fourth and fifth aspects cells are provided. Said cells may be any cells as described earlier herein, preferably they are eukaryotic cells, more preferably human cells. Said cells comprise a nucleic acid construct comprising a promoter and/or enhancer sequence operably linked to a nucleic acid sequence encoding a polypeptide. The promoter and/or enhancer sequence is inducible by an olfactory receptor, as described earlier herein. The encoded polypeptide may preferably confer resistance to an antibiotic, as described earlier herein. The encoded polypeptide may be a reporter polypeptide, as described earlier herein. Said cells comprise a second nucleic acid construct comprising a nucleic acid molecule encoding an olfactory receptor, as described earlier herein. Preferably, the nucleic acid construct comprising a promoter and/or enhancer sequence operably linked to a nucleic acid sequence encoding a polypeptide and the second nucleic acid construct comprising a nucleic acid molecule encoding an olfactory receptor are fused so as to constitute a single nucleic acid construct, preferably a single plasmid.

The cells of step A) of the fourth and fifth aspects may optionally comprise additional nucleic acid constructs and/or nucleotide sequences, as described earlier herein, preferably they comprise nucleic acid constructs comprising nucleic acid sequences encoding accessory molecules needed for functional expression of an olfactory receptor in a cell as described earlier herein, more preferably nucleic acid sequences encoding for the V2271 variant of the human RTP1S and/or the L220R variant of the human RTP2 (SEQ ID NO: 17 or 18).

Step A) of the fourth and fifth aspects may involve the culturing of cells. Cell culturing may be carried out using a culture medium comprising suitable nutrients, such as carbon and nitrogen sources and additional compounds such as inorganic salts, trace elements, and vitamins. The skilled person understands that suitable nutrients (as well as culture conditions such as temperature, pH, CO₂ levels, and the like) will vary depending on the cultured cell. Suitable culture media and culture conditions are available from commercial suppliers and further discussed in standard handbooks and in cell line information found in publicly available culture collections, e.g. the American Type Culture Collection (VA, USA). A non-limiting example of a suitable culture medium is Dubelcco's Modified Eagle medium (DMEM, commercially available by e.g. ThermoFisher Scientific, MA, USA).

Cell culturing may be performed at a temperature value that may vary depending on the cultured cell. In some embodiments wherein human cells are cultured, cell culturing is preferably performed at a temperature range of from 34 to 39° C., more preferably at a temperature range of from 35 to 38° C., even more preferably at a temperature range of from 36 to 37° C. In some most preferred embodiments wherein human cells are cultured, a temperature value of 37° C. or about 37° C. is used.

Cell culturing may be performed at a pH value that may vary depending on the cultured cell. In some embodiments wherein human cells are cultured, cell culturing is preferably performed at a pH value range of from 7.0 to 7.7, more preferably at a pH value range of from 7.2 to 7.6, even more preferably at a pH value range of from 7.4 to 7.5. In some most preferred embodiments wherein human cells are cultured, a pH value of 7.5 or about 7.5 is used.

Cell culturing may be performed at a CO₂% value that may vary depending on the cultured cell and culture medium. The skilled person understands that supply of exogenous CO₂, for example by flushing the cell culture with a CO₂-air mixture, may be required in some cases where, for example, media buffered with a CO₂-bicarbonate based buffer are used. In some embodiments wherein human cells are cultured and exogenous CO₂ is supplied, said CO₂ may preferably be from 4 to 10% in air, more preferably from 4 to 7% in air, even more preferably from 5 to 6% in air. In some more preferred embodiments wherein human cells are cultured, a CO₂ value of 5% or about 5% in air is used.

Cell culturing duration may vary depending on the cultured cell. In some embodiments, said duration may be at least 30 min, at least 1h, at least 2h, at least 3h, at least 4h, at least 5 h, at least 6h, at least 7h, at least 8h, at least 9h, at least 10h, at least 11h, at least 12h, at least 13h, at least 14h, at least 15h, at least 16h, at least 17h, at least 18h, at least 19h, at least 20h, at least 21h, at least 22h, at least 23h, at least 24h, at least at least 31h, at least 38h, at least 2 days, at least 3 days, at least 4 days, at least 5 days, at least 6 days, or at least a week.

Step A) of the method of the fifth aspect comprises a mutagenesis step. Said mutagenesis step may include any step wherein the cells are genetically modified, for example by the introduction of one or more nucleotide mutations, substitutions, insertions, and/or deletions in its genome, and/or introduction of additional nucleic acid constructs as described earlier herein. In some embodiments, the mutagenesis step is carried out using insertional mutagenesis, wherein a nucleic acid sequence is inserted in the genome of the cells using plasmids, linearized DNA sequences, transposons, retroviruses, lentiviruses or CRISPR-Cas-mediated recombination, preferably wherein the inserted nucleic acid sequence comprises an enhancer and/or promoter sequence suitable for activation of expression of endogenous genes, as described earlier herein. The insertion site of the inserted nucleic acid sequence may optionally be mapped and/or identified in the selected or sorted cells using genomic mapping and/or sequencing methods as described later herein. The skilled person understands that the mutagenesis step may be repeated multiple times. In some embodiments, the mutagenesis step is carried out using CRISPR-Cas-mediated mutagenesis, using CRISPR interference or CRISPR activation. The application of a mutagenesis step to the cells may allow for improved functional expression. For example, and because the mutagenesis step is not restricted to the nucleic acid molecule encoding the olfactory receptor, it may be particularly useful in selecting cells comprising further genetic and/or epigenetic modifications in nucleic acid molecules encoding olfactory receptors, accessory molecules, and/or other genomic regions which allow improved functional expression of olfactory receptors, particularly in the case of olfactory receptors which are difficult to express using conventional methods. The modifications may then be mapped and/or identified using genomic mapping, epigenetics assays, and/or sequencing methods, as described later herein.

In step B) of the method of the fourth and fifth aspects the cells are cultured in the presence of the ligand of the olfactory receptor. Culture media, conditions and duration correspond to the ones described in step A), a difference being the addition of the ligand. The ligand may be added in an existing culture, or alternatively the culture medium of an existing culture may be replaced by fresh culture medium comprising said ligand. Suitable ligands may be selected from any chemical compound known in the art that is able to activate an olfactory receptor (alternatively referred to as “aroma compounds” or “odorants”), which are discussed in standard handbooks such as Buettner (2017), Springer Handbook of Odor, Springer International publishing (CH), incorporated herein by reference in its entirety. Non-limiting examples of suitable ligands include esters (e.g. geranyl acetate, methyl formate, methyl acetate, methyl propionate, methyl butyrate, ethyl acetate, ethyl butyrate, isoamyl acetate, pentyl butyrate, pentyl penthanoate, octyl acetate, benzyl acetate, methyl anthranilate, hexyl acetate), linear terpenes (e.g. myrcene, geraniol, nerol, citral, citronellal, citronellol, linalool, nerolidol, ocimene), cyclic terpenes (limonene, camphor, methol, carvone, terpineol, alpha-lonone, thujone, eucalyptol, jasmine), aromatic compounds (e.g. benzaldehyde, eugenol, isoeugenol, cinnamaldehyde, ethyl maltol, ethyl vanillin, anisole, anethole, estragole, thymol), amines (e.g. trimethylamine, putrescine, cadaverine, pyridine, indole, skatole), alcohols (e.g. furaneol, 1-hexanol, ethanol), aldehydes (e.g. acetaldehyde, hexanal, furfural, hexyl cinnamaldehyde, isovaleraldehyde, anisic aldehyde, cuminaldehyde), ketones (e.g. dihydrojasmone, 2-acetyl-1-pyrroline, 6-acetyl-2,3,4,5-tetrahydropyridine), lactones (e.g. gamma-decalactone, gamma-nonalactone, delta-octalactone, jasmine lactone, massoia lactone, wine lactone, sotolon), thiols (e.g. thioacetone, allyl thiol, ethanethiol, 2-methyl-2-propanethiol, butane-1-thiol, mercaptan, methanethiol, furan-2-ylmethanethiol, benzyl mercaptan), musks (e.g. nitromusks, polycyclic musks, macrocyclic musks, linear/alicyclic musks, musk ketone, musk ambrette, musk moskene, musk tibetene, musk xylene), cresols (e.g. vanilla cresol (ultravanil)), propenyl guaethol (vanitrope), and the like.

The skilled person understands that the amount of ligand required for the activation of an olfactory receptor may vary depending on the olfactory receptor and the ligand's ability to physically associate with said olfactory receptor. A ligand may be considered to be “of” a given olfactory receptor (i.e. specific for that receptor) if it can physically associate (i.e. bind to) with said receptor at an EC₅₀ value of 1 mM or less, typically at an EC₅₀ value between 10 nM and 1 mM. EC₅₀ in the context of ligands of olfactory receptors refers to that concentration of a ligand at which a given activation of an olfactory receptor is 50% of the maximum for that olfactory receptor, measurable using methods as described elsewhere herein.

In some embodiments, the ligand may be present in the culture at a concentration value from 0.1 nM to 1 mM, 1 nM to 1 mM, 10 nM to 1 mM, from 100 nM to 500 μM, from 250 nM to 100 μM, from 500 nM to 50 μM, or from 10 μM to 30 μM.

The skilled person understands that the duration of cells culturing in the presence of the ligand of the olfactory receptor may vary depending on the olfactory receptor and the ligand's ability to physically associate with said olfactory receptor. In some embodiments, cells are cultured for at least at least 30 min, at least 1h, at least 2h, at least 3h, at least 4h, at least 5 h, at least 6 h, at least 7h, at least 8h, at least 9h, at least 10h, at least 11h, at least 12h, at least 13h, at least 14 h, at least 15h, at least 16h, at least 17h, at least 18h, at least 19h, at least 20h, at least 21h, at least 22h, at least 23h, or at least 24h.

In step C1) of the method of the fourth and fifth aspects cells functionally expressing the olfactory receptor are selected by cell culturing in the presence of the antibiotic and the ligand. The presence of the antibiotic in the culture exposes the cells to selective conditions (i.e. it applies selective pressure). As discussed earlier herein, only cells which are able to functionally express the olfactory receptor will exhibit antibiotic resistance and will be able to survive and/or grow, facilitating their selection. Culture media, conditions and duration correspond to the ones described in step A) or B), a difference being the addition of the antibiotic (compared to step B). The antibiotic may be added in an existing culture, or alternatively the culture medium of an existing culture may be replaced by fresh culture medium comprising said antibiotic.

The skilled person understands that the choice of the antibiotic will vary depending on the antibiotic resistance conferred by the encoded polypeptide. Any antibiotic may be contemplated, including, but not limited to, compounds selected from penicillins (β-lactams), aminonucleosides, nucleoside analogues, tetracyclines, cephalosporins, quinolones, lincomycins, macrolides, sulphonamides, polypeptides, glycopeptides, lipoglycopeptides, aminoglycosides, fluoroquinolones, monobactams, oxazolidinones, streptogramins, rifamycins, carbapenems, chloramphenicol, clindamycin, daptomycin, fosfomycin, lefamulin, metronidazole, mupirocin, nitrofurantoin, tigecycline, puromycin, hygromycin B (hygrovetine), geneticin (G418), bleomycin, zeocin, and blasticidin. In some embodiments, the antibiotic is selected from puromycin, hygromycin B (hygrovetine), geneticin (G418), zeocin, and blasticidin. In some embodiments, the antibiotic is puromycin or blasticidin (blasticidin S), preferably blasticidin.

The skilled person understands that the amount of the antibiotic present in the culture in order to achieve selective conditions may vary depending on the antibiotic and/or the cell. To select an appropriate amount that will inhibit the survival and/or growth of cells not functionally expressing the olfactory receptor, the minimum inhibitory concentration (MIC) of a given antibiotic for a given cell may be used. The term “minimum inhibitory concentration” refers to the minimum concentration of an antibiotic which prevents visible growth of a given cell. MIC values of antibiotics for given cells are available in public databases and may be further determined using methods known in the art such as described in standard handbooks like Schwalbe R. et al., Antimicrobial susceptibility testing protocols, Boca Raton: CRC Press (2007), incorporated herein by reference in its entirety, and/or commercially available kits and protocols such as ETEST® (Biomerieux, NC, USA). Alternatively, the concentration of an antibiotic that results in a reduction of cell viability by 50% (EC₅₀ of a given antibiotic) may be used. Alternatively, to select an appropriate antibiotic amount that will inhibit the survival and/or growth of cells not functionally expressing the olfactory receptor, exposure of cells to the antibiotic followed by incubation with commercially available cell viability reagents, such as PrestoBlue® (ThemoFisher Scientific, MA, USA) following the supplier's protocols, may be used. Examples of the application of such a reagent to determine cell viability following exposure to an antibiotic is further provided in the experimental section herein.

In some embodiments, an antibiotic may be present in the culture at a concentration value from 10 ng/ml to 1 mg/ml, from 15 ng/ml to 500 μg/ml, from 20 ng/ml to 250 μg/ml, from 25 ng/ml to 125 μg/ml, from 50 ng/ml to 100 μg/ml, from 0.1 μg/ml to 90 μg/ml, from 0.5 μg/ml to 80 μg/ml, from 1 μg/ml to 70 μg/ml, from 2 μg/ml to 60 μg/ml, from 3 μg/ml to 50 μg/ml, from 4 μg/ml to 30 μg/ml, or from 5 μg/ml to 20 μg/ml. In some embodiments, cell culturing in the presence of the antibiotic may have a duration of at least 30 min, at least 1h, at least 2h, at least 3h, at least 4h, at least 5h, at least 6h, at least 7h, at least 8h, at least 9h, at least 10h, at least 11h, at least 12h, at least 13h, at least 14h, at least 15h, at least 16h, at least 17h, at least 18h, at least 19h, at least 20h, at least 21h, at least 22h, at least 23h, at least 24h, at least at least 31h, at least 38h, at least 2 days, at least 3 days, at least 4 days, at least 5 days, at least 6 days, at least a week, at least two weeks, at least three weeks, or more.

In some embodiments, functional expression of an olfactory receptor by a cell cultured in the presence of an antibiotic and the ligand results in an viability increase of said cell of at least 10%, at least 20%, at least 30%, at least 40%, or at least 50%, relative to a comparable cell not expressing the olfactory receptor or to a comparable cell cultured only in the presence of the antibiotic. In some embodiments, the viability increase of said cell is at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 25-fold, at least 30-fold, at least 35-fold, at least 40-fold, at least 45-fold, at least 50-fold, or at least 100-fold, relative to a comparable cell not expressing the olfactory receptor or to a comparable cell cultured only in the presence of the antibiotic.

In some embodiments, the concentration of an antibiotic that is required to decrease cell viability by 50% (EC₅₀ of a given antibiotic) of a cell functionally expressing an olfactory receptor cultured in the presence of the antibiotic and the ligand is increased by at least 10%, at least 20%, at least 30%, at least 40%, or at least 50%, relative to the case of a comparable cell not expressing the olfactory receptor or of a comparable cell cultured only in the presence of the antibiotic. In some embodiments, the required concentration of an antibiotic is increased by at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 25-fold, at least 30-fold, at least 35-fold, at least 40-fold, at least 45-fold, at least 50-fold, or at least 100-fold, relative to the case of a comparable cell not expressing the olfactory receptor or of a comparable cell cultured only in the presence of the antibiotic.

In some cases, % viability may be alternatively or additionally determined relative to the viability of controls with no cells (corresponding to 0% viability) and/or the viability of cells treated with olfactory ligand only (without the antibiotic, corresponding to 100% viability).

Step C1) of the method of the fourth and fifth aspects may optionally involve sub-culturing of the cells under selective conditions (i.e. selective pressure) over multiple generations (evolutionary engineering, described earlier herein). Said sub-culturing involves the removal of (nutrient) depleted culture medium following the growth of the cells, and its replacement with medium comprising the same or different ligand and/or antibiotic concentrations. Nutrient depletion of culture medium may be assessed by the skilled person using standard methods in the art, for example HPLC. Continuous sub-culturing, i.e. the constant supply and removal of culture medium so as to achieve a steady state in the culture, may be alternatively applied. Because the selectable phenotype (i.e. antibiotic resistance arising from olfactory receptor activation) is linked to the growth of the cells, said sub-culturing may be advantageous in selecting cells with improved functional expression of olfactory receptors, as said cells will have a selective growth advantage and will be enriched in the culture, for example in cases wherein baseline functional expression of a given olfactory receptor is too low to be detectable. Said enrichment may be further enhanced by the application of a progressively increased antibiotic concentration in the culture medium, a progressively decreased ligand concentration in the culture medium or a combination of the two. Such an approach is particularly useful in selecting cells comprising further genetic and/or epigenetic modifications in nucleic acid molecules encoding olfactory receptors, accessory molecules, and/or other genomic regions which allow improved functional expression of olfactory receptors, particularly in the case of olfactory receptors which are difficult to express using conventional methods. The modifications may then be mapped and/or identified using genomic mapping, epigenetics assays, and/or sequencing methods, as described later herein.

In step C2) of the method of the fourth and fifth aspects, cells functionally expressing the olfactory receptor are screened by the detection and sorting out of cells expressing the reporter polypeptide in the presence of the ligand. As discussed earlier herein, only cells which are able to functionally express the OR will express the reporter polypeptide, which allows for their detection and sorting out. Said cells may be detected directly or indirectly, as discussed earlier herein. Any reporter polypeptide and detection method as discussed earlier herein may be used. The skilled person understands that the exact method of detection and sorting out will depend on the reporter polypeptide and the utilized instrument. Sorted individual cells may be subsequently cultured. Any genetic and/or epigenetic modifications in nucleic acid molecules encoding olfactory receptors, accessory molecules, and/or other genomic regions which allow functional expression of olfactory receptors may then be mapped and/or identified using genomic mapping, epigenetics assays, and/or sequencing methods, as described later herein.

As a non-limiting example, in a case wherein GFP is used as a reporter polypeptide, a cell culture coming from step B) may be loaded in a commercially available fluorescence-activated cell sorter (e.g. BD-FACS™ available from BD, NJ, USA). The cell culture may then be exposed to a light wavelength of about 488 nm, and GFP may be optimally detected at a wavelength of 510 nm. The cells expressing GFP can then be screened and sorted out following the manufacturer's protocol.

Genetic and/or epigenetic modifications in nucleic acid molecules encoding olfactory receptors, accessory molecules, and/or other genomic regions which allow functional or improved functional expression of olfactory receptors may be mapped and/or identified using genomic mapping, epigenetics assays, and/or sequencing methods. Said mapping and/or identification may take place after any step of the methods of the fourth and fifth aspects, preferably it takes place after step C1) or C2).

In some embodiments of the methods of the fourth and fifth aspects, the genetic and/or epigenetic modifications comprised in nucleic acid molecules encoding olfactory receptors, accessory molecules, and/or other genomic regions which allow functional or improved functional expression of olfactory receptors by the selected or sorted cells are mapped and identified.

Mapping refers to the identification of the location of a nucleic acid sequence such as a gene as well as the distance between nucleic acid sequences in a cell's genome. Mapping may be particularly useful in embodiments wherein insertional mutagenesis is carried out in step A) of the method of the fifth aspect as described earlier herein, as it allows for the identification of the insertion site of the inserted nucleic acid sequence in the selected or sorted cells. Mapping may be performed via genetic mapping, i.e. mapping using genetic linkage information based on genetic markers, physical mapping or a combination of both. Mapping methods are known in the art and discussed in standard handbooks like Brown, Genomes, 4th edition, Garland Science, NY, USA (2017), incorporated herein by reference in its entirety. Non-limiting examples of mapping methods include circular PCR, comprising digestion of chromosomal DNA followed by ligation to form circular DNA followed by its PCR amplification, and chromosomal walking, for example comprising digestion of chromosomal DNA followed by ligation with one or more adapters followed by nested PCR directed to the one or more adapters and the inserted sequence.

Identification of genetic modifications may be performed using any nucleic acid sequencing method known to the skilled person. Non-limiting examples include Sanger sequencing, single-molecule real-time sequencing, ion torrent sequencing, pyrosequencing, Illumina-sequencing, combinatorial probe anchor synthesis, sequencing by ligation (SOLID sequencing), Nanopore sequencing, GenapSys sequencing, and the like. Sequencing sample preparation, instruments, and protocols are discussed in standard handbooks like Head, Ordoukhanian and Salomon (Eds), Next Generation Sequencing: Methods and Protocols, Humana Press, NJ, USA (2018), incorporated herein by reference in its entirety, with many being commercially available, e.g. from Illumina (CA, USA), Pacific Biosciences (CA, USA), and others.

Epigenetic modifications of nucleic acid molecules encoding olfactory receptors, accessory molecules, or other genomic regions as described earlier herein may be identified using any standard epigenetics assay known in the art, such as described in standard handbooks and publications like Tollefsbol, Handbook of Epigenetics: The New Molecular and Medical Genetics, 2nd Edition, Academic Press, USA (2017), and DeAngelis and Woodrow (2008) Mol Biotechnol 38 (2): 179-183, both of which are incorporated herein by reference in their entireties. Non-limiting examples of epigenetic assays are chromatin immunoprecipitation (ChIP, together with its large-scale variants ChIP-on-chip and ChIP-Seq), fluorescent in situ hybridization, methylation-sensitive restriction digestion, DNA adenine methyltransferase identification (DamID), bisulfite sequencing, RNA Immunoprecipitation (RIP), cross-linking immunoprecipitation. Many epigenetics assays are commercially available, for example the epigenetics assays and kits offered from Abcam (Cambridge, UK).

The methods of the invention are further suitable for identification of novel cognate receptor-ligand pairs. In particular, for many ligands of interest the cognate olfactory receptor receptors are either not known (“orphan receptors”), or not well-characterized, e.g. in their affinities for the ligand of interest.

Accordingly, in a sixth aspect, the invention provides a method for identifying an olfactory receptor binding to a given ligand, said method comprising the following steps:
A) Providing a heterogeneous population of cells as described earlier herein, wherein said cells comprise a nucleic acid construct comprising a promoter and/or enhancer sequence operably linked to a nucleic acid sequence encoding a polypeptide, wherein said encoded polypeptide confers resistance to an antibiotic or is a reporter polypeptide, wherein said promoter and/or enhancer is inducible by an olfactory receptor, preferably wherein said promoter and/or enhancer sequence comprises one or multiple copies of a cAMP responsive element (CRE), a half CRE, or an NFAT responsive element (NFAT-RE), and a second nucleic acid construct comprising a nucleic acid molecule encoding an olfactory receptor, wherein the olfactory receptor encoded by the nucleic acid molecule comprised in said second nucleic acid construct comprised in at least one of the cells is distinct from the olfactory receptor encoded by the nucleic acid molecule comprised in the second nucleic acid construct in at least one of the other cells of said population,
B) Culturing said population of cells in the presence of said given ligand,
C1) Selecting for cells functionally expressing the olfactory receptor binding to said given ligand by culturing them in the presence of the antibiotic and the ligand, or
C2) Screening for cells functionally expressing an olfactory receptor binding to said given ligand by detecting and sorting out cells expressing the reporter polypeptide in the presence of the ligand,
D) Determining the nucleotide sequence encoding the receptor in the selected or sorted cells.

In step A), a nucleic acid sequence encoding a polypeptide conferring resistance to an antibiotic is preferred, preferably the nucleic acid sequence is a puromycin-N-acetyltransferase gene or a blasticidin-S deaminase gene. Among steps C1) and C2), step C1) is preferred.

The description of step A) of the fourth aspect provided earlier herein also applies to step A) of the sixth aspect, with the difference that in step A) of the sixth aspect a population of cells is provided. Said population is heterogeneous (mixed) as described earlier herein. Preferably, the nucleic acid construct comprising a promoter and/or enhancer sequence operably linked to a nucleic acid sequence encoding a polypeptide and the second nucleic acid construct comprising a nucleic acid molecule encoding an olfactory receptor are fused so as to constitute a single nucleic acid construct, preferably a single plasmid.

The provision of a heterogeneous population may be particularly advantageous, as it allows for high-throughput identification of an olfactory receptor binding to a ligand of interest, without requiring prior knowledge of said olfactory receptor characteristics, or of the required accessory molecules or of any specific genetic and/or epigenetic modifications that may be required to achieve functional expression of said olfactory receptor.

The description of step B) of the fourth and fifth aspects provided earlier herein also applies to step B) of the sixth aspect, with a difference that in step B) of the sixth aspect the olfactory receptor to which the given ligand is binding to is not yet identified. Any ligand of interest, culturing media, and culturing conditions as described earlier herein may be chosen and utilized.

The description of steps C1) and C2) of the fourth and fifth aspects provided earlier herein also applies to steps C1) and C2) of the sixth aspect, with a difference that, since in steps C1) and C2) of the sixth aspect the olfactory receptor binding to the given ligand is not yet identified, steps C1) and C2) of the sixth aspect simultaneously allow for selecting or screening and sorting out of cells functionally expressing the olfactory receptor the given ligand binds to. In step C1) of the sixth aspect, and as described earlier herein, because the selectable phenotype (i.e. antibiotic resistance arising from olfactory receptor activation) is linked to the survival and/or growth of the cells functionally expressing the olfactory receptor the given ligand binds to, cell sub-culturing under selective pressure may be advantageous to select cells with improved functional expression of olfactory receptors, as said cells will have a selective growth advantage and will be enriched in the culture.

Accordingly, in some embodiments of the methods of the fourth, fifth, and sixth aspects, step C1) additionally comprises a sub-culturing step wherein cells with improved functional expression of the olfactory receptor are enriched in a culture.

In step D) of the method of the sixth aspect, the nucleotide sequence encoding the olfactory receptor in the selected or sorted cells is determined. Thus, said receptor may be identified (“de-orphanized”) and the cognate olfactory receptor-ligand relationship may be resolved. Optionally, nucleotide sequences encoding accessory molecules that may be required to achieve functional expression of the olfactory receptor are determined. Optionally, any specific genetic and/or epigenetic modifications that may be required to achieve functional expression of the olfactory receptor are identified.

Suitable nucleic acid sequences encoding a polypeptide conferring resistance to an antibiotic have been discussed earlier herein. In some embodiments, the nucleic acid sequence is a puromycin-N-acetyltransferase gene or a blasticidin-S deaminase gene, preferably a blasticidin-S deaminase gene.

Non-limiting examples of olfactory receptors, optionally comprising genetic modifications as described above, that may be identified using the methods of the invention comprise, essentially consist of, or consist of, preferably comprise, a polypeptide comprising the amino acid sequence of SEQ ID NOs: 20, 36-62.

Determination of the sequence of an olfactory receptor or an accessory molecule or identification of any genetic and/or epigenetic modification as may be performed by any genomic mapping, epigenetics assay, and/or sequencing method discussed earlier herein.

### Olfactory Receptor

The nucleic acid constructs, cells, and methods of the invention enable the identification and/or selection of olfactory receptors and/or accessory molecules comprising genetic and/or epigenetic modifications required for functional or improved functional expression of said olfactory receptors.

Accordingly, in a seventh aspect, the invention provides a variant olfactory receptor and/or accessory molecule. Definitions of “variant”, “functional expression” and “improved functional expression” have been provided earlier herein. The variant olfactory receptor may be functionally expressed in a cell as described earlier herein, whereas the naturally-occurring sequence is not functionally expressed by said cell. The variant olfactory receptor may have improved functional expression in a cell relative to the functional expression of the naturally-occurring sequence in said cell. Genetic modifications in the C-terminus and/or N-terminus end of olfactory receptors may be advantageous, as said regions are typically not responsible for ligand selectivity, but are typically important for OR trafficking and/or incorporation to the cell-surface membrane. In some embodiments, the olfactory receptor comprises a genetic modification in the N-terminus. In some embodiments, the olfactory receptor comprises a genetic modification in the C-terminus. In some embodiments, the olfactory receptor comprises a genetic modification in the N-terminus and a genetic modification in the C-terminus. Said genetic modifications include, but are not limited to, amino acid insertions, deletions and/or substitutions, arising from nucleotide insertions, deletions and/or substitutions in the nucleotide sequences encoding said olfactory receptors, as described earlier herein. In some embodiments, the olfactory receptor is synthetic.

Variants of the human OR5A2 receptor (NCBI Genbank ID: 219981, SEQ ID NO: 19) are particularly advantageous. The wild-type sequence of human OR5A2 encodes a difficult-to-express receptor, which typically cannot be functionally expressed in cells expressing the chaperone RTP1S and RTP2. It requires unknown accessory factors for functional expression. OR5A2 was postulated to be a musk receptor in WO2019110630A1, incorporated herein by reference in its entirety. As demonstrated in the experimental section herein, the present inventors were able to select and isolate a variant of OR5A2 (SEQ ID NO: 20) which may be functionally expressed in cells, such as, but not limited to, HEK293T cells. Said variant comprises a modified C-terminus (SEQ ID NO: 62).

Therefore, in an aspect the invention relates to an olfactory receptor, whose amino acid sequence comprises an amino acid sequence having at least 60%, at least 70%, at least 80%, at least 90%, at least 95%, at least 96%, at least 97%, at least 98%, at least 99%, or 100%, identity or similarity with SEQ ID NO: 62, preferably wherein SEQ ID NO: 62 is located at the C-terminus of the olfactory receptor.

In a preferred embodiment, the amino acid sequence of an olfactory receptor comprises, consists essentially of, or consists of, preferably comprises, an amino acid sequence having at least 60%, at least 70%, at least 80%, at least 90%, at least 95%, at least 96%, at least 97%, at least 98%, at least 99%, or 100%, identity or similarity with SEQ ID NO: 20.

In a preferred embodiment, the amino acid sequence of an olfactory receptor comprises, consists essentially of, or consists of, preferably comprises, an amino acid sequence having at least 60%, at least 70%, at least 80%, at least 90%, at least 95%, at least 96%, at least 97%, at least 98%, at least 99%, or 100%, identity or similarity with SEQ ID NO: 20 and comprises an amino acid sequence having at least 60%, at least 70%, at least 80%, at least 90%, at least 95%, at least 96%, at least 97%, at least 98%, at least 99%, or 100%, identity or similarity with SEQ ID NO: 62, preferably wherein SEQ ID NO: 62 is located at the C-terminus of the olfactory receptor.

Identity or similarity may be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

Such olfactory receptor is assumed to be functionally expressed in a cell. In an embodiment, such functional expression is improved by comparison with the expression of a control or reference olfactory receptor. The control or reference olfactory receptor may in some embodiments be OR5A2, for example human OR5A2.

Accordingly, in a further aspect the invention relates to an olfactory receptor whose amino acid sequence comprises a polypeptide which is encoded by a nucleic acid molecule encoding an amino acid sequence represented by an amino acid sequence having at least 60%, at least 70%, at least 80%, at least 90%, at least 95%, at least 96%, at least 97%, at least 98%, at least 99%, or 100%, identity or similarity with SEQ ID NO: 62, preferably wherein SEQ ID NO: 62 is located at the C-terminus of the olfactory receptor.

In a preferred embodiment, the amino acid sequence of an olfactory receptor is encoded by a nucleic acid molecule, nucleic acid molecule encoding an amino acid sequence comprising, consisting essentially of, or consisting of, preferably comprising, an amino acid sequence having at least 60% at least 70%, at least 80%, at least 90%, at least 95%, at least 96%, at least 97%, at least 98%, at least 99%, or 100%, identity or similarity with SEQ ID NO: 20.

In a preferred embodiment, the amino acid sequence of an olfactory receptor is encoded by a nucleic acid molecule, nucleic acid molecule encoding an amino acid sequence comprising, consisting essentially of, or consisting of, preferably comprising, an amino acid sequence having at least 60%, at least 70%, at least 80%, at least 90%, at least 95%, at least 96%, at least 97%, at least 98%, at least 99%, or 100%, identity or similarity with SEQ ID NO: 20 and comprising an amino acid sequence having at least 60%, at least 70%, at least 80%, at least 90%, at least 95%, at least 96%, at least 97%, at least 98%, at least 99%, or 100%, identity or similarity with SEQ ID NO: 62, preferably wherein SEQ ID NO:62 is located at the C-terminus of the olfactory receptor.

Identity or similarity may be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

TABLE 1
List of sequences

| | | | Type |
|---|---|---|---|
| SEQ ID | | | (DNA |
| NO | Name | Sequence | or aa) |
| 1 | CRE | TGACGTCA | DNA |
| 2 | half CRE | TGACG | DNA |
| 3 | CAMP-responsive | GCACCAGACAGTGACGTCAGCTGCCAGATCCCATGGC | DNA |
| | promoter_1 | CGTCATACTGTGACGTCTTTCAGACACCCCATTGACG | |
| | | TCAATGGGAGAAC | |
| 4 | CAMP-responsive | GCACCAGACAGTGACGTCAGCTGCCAGATCCCATGGC | DNA |
| | promoter_2 | CGTCATACTGTGACGTCTTTCAGACACCCCATTGACG | |
| | | TCAATGGGAGAACAGATCTGGCCTCGGCGGCCAAGCT | |
| | | TAGACACTAGAGGGTATATAATGGAAGCTCGACTTCC | |
| | | AG | |
| 5 | NFAT-RE | GGAAAA | DNA |
| 6 | NFAT-responsive | GGAGGAAAAACTGTTTCATACAGAAGGCGTGGAGGAA | DNA |
| | promoter | AAACTGTTTCATACAGAAGGCGTGGAGGAAAAACTGT | |
| | | TTCATACAGAAGGCGT | |
| 7 | pac | ATGACCGAGTACAAGCCCACGGTGCGCCTCGCCACCC | DNA |
| | | GCGACGACGTCCCCCGGGCAGTACGCACCCTCGCCGC | |
| | | CGCGTTCGCCGACTACCCCGCCACGCGCCACACCGTC | |
| | | GATCCAGACCGCCACATCGAGCGGGTCACCGAGCTGC | |
| | | AAGAACTCTTCCTCACGCGCGTCGGGCTCGACATCGG | |
| | | CAAGGTGTGGGTCGCGGACGACGGCGCCGCGGTGGCG | |
| | | GTCTGGACCACGCCGGAGAGCGTCGAAGCGGGGGCGG | |
| | | TGTTCGCCGAGATCGGCCCGCGCATGGCCGAGTTGAG | |
| | | CGGTTCCCGGCTGGCCGCGCAGCAACAGATGGAAGGC | |
| | | CTCCTGGCGCCGCACCGGCCCAAGGAGCCCGCGTGGT | |
| | | TCCTGGCCACCGTCGGCGTCTCGCCCGACCACCAGGG | |
| | | CAAGGGTCTGGGCAGCGCCGTCGTGCTCCCCGGAGTG | |
| | | GAGGCGGCCGAGCGCGCCGGGGTGCCCGCCTTCCTGG | |
| | | AGACCTCCGCGCCCCGCAACCTCCCCTTCTACGAGCG | |
| | | GCTCGGCTTCACCGTCACCGCCGACGTCGAGGTGCCC | |
| | | GAAGGACCGCGCACCTGGTGCATGACCCGCAAGCCCG | |
| | | GTGCCTGA | |
| 8 | hph | ATGAAGAAGCCCGAACTCACCGCTACCAGCGTTGAAA | DNA |
| | | AATTTCTCATCGAGAAGTTCGACAGTGTGAGCGACCT | |
| | | GATGCAGTTGTCGGAGGGCGAAGAGAGCCGAGCCTTC | |
| | | AGCTTCGATGTCGGCGGACGCGGCTATGTACTGCGGG | |
| | | TGAATAGCTGCGCTGATGGCTTCTACAAAGACCGCTA | |
| | | CGTGTACCGCCACTTCGCCAGCGCTGCACTACCCATC | |
| | | CCCGAAGTGTTGGACATCGGCGAGTTCAGCGAGAGCC | |
| | | TGACATACTGCATCAGTAGACGCGCCCAAGGCGTTAC | |
| | | TCTCCAAGACCTCCCCGAAACAGAGCTGCCTGCTGTG | |
| | | TTACAGCCTGTCGCCGAAGCTATGGATGCTATTGCCG | |
| | | CCGCCGACCTCAGTCAAACCAGCGGCTTCGGCCCATT | |
| | | CGGGCCCCAAGGCATCGGCCAGTACACAACCTGGGGG | |
| | | GATTTCATTTGCGCCATTGCTGATCCCCATGTCTACC | |
| | | ACTGGCAGACCGTGATGGACGACACCGTGTCCGCCAG | |
| | | CGTAGCTCAAGCCCTGGACGAACTGATGCTGTGGGCC | |
| | | GAAGACTGTCCCGAGGTGCGCCACCTCGTCCATGCCG | |
| | | ACTTCGGCAGCAACAACGTCCTGACCGACAACGGCCG | |
| | | CATCACCGCCGTAATCGACTGGTCCGAAGCTATGTTC | |
| | | GGGGACAGTCAGTACGAGGTGGCCAACATCTTCTTCT | |
| | | GGCGGCCCTGGCTGGCTTGCATGGAGCAGCAGACTCG | |
| | | CTACTTCGAGCGCCGGCATCCCGAGCTGGCCGGCAGC | |
| | | CCTCGTCTGCGAGCCTACATGCTGCGCATCGGCCTGG | |
| | | ATCAGCTCTACCAGAGCCTCGTGGACGGCAACTTCGA | |
| | | CGATGCTGCCTGGGCTCAAGGCCGCTGCGATGCCATC | |
| | | GTCCGCAGCGGGGCCGGCACCGTCGGTCGCACACAAA | |
| | | TCGCTCGCCGGAGCGCAGCCGTATGGACCGACGGCTG | |
| | | CGTCGAGGTGCTGGCCGACAGCGGCAACCGCCGGCCC | |
| | | AGTACACGACCGCGCGCTAAGGAGGTAGGTCGAGTTT | |
| | | AA | |
| 9 | Homo sapiens | MRPQILLLLALLTLGLA | aa |
| | LUCY-tag | | |
| 10 | Mus musculus | MSHQILLLLALLTLGLA | aa |
| | LUCY-tag | | |
| 11 | FLAG-tag | MDYKDDDDK | aa |
| 12 | rho-tag | MNGTEGPNFYVPFSNKTGVV | aa |
| 13 | mmLucy-FLAG- | ATGAGCCACCAGATCCTGCTGCTCCTGGCCCTGCTGA | DNA |
| | rho | CCCTAGGCCTGGCTGATTACAAGGACGACGACGATAA | |
| | | GATCGAATTGATGAACGGGACCGAGGGCCCAAACTTC | |
| | | TACGTGCCTTTCTCCAACAAGACGGGCGTGGTGGAAT | |
| | | TC | |
| 14 | mmLucy-FLAG- | MSHQILLLLALLTLGLADYKDDDDKIELMNGTEGPNF | aa |
| | rho | YVPFSNKTGVVEF | |
| 15 | CMV promoter | GTTGACATTGATTATTGACTAGTTATTAATAGTAATC | DNA |
| | | AATTACGGGGTCATTAGTTCATAGCCCATATATGGAG | |
| | | TTCCGCGTTACATAACTTACGGTAAATGGCCCGCCTG | |
| | | GCTGACCGCCCAACGACCCCCGCCCATTGACGTCAAT | |
| | | AATGACGTATGTTCCCATAGTAACGCCAATAGGGACT | |
| | | TTCCATTGACGTCAATGGGTGGAGTATTTACGGTAAA | |
| | | CTGCCCACTTGGCAGTACATCAAGTGTATCATATGCC | |
| | | AAGTACGCCCCCTATTGACGTCAATGACGGTAAATGG | |
| | | CCCGCCTGGCATTATGCCCAGTACATGACCTTATGGG | |
| | | ACTTTCCTACTTGGCAGTACATCTACGTATTAGTCAT | |
| | | CGCTATTACCATGGTGATGCGGTTTTGGCAGTACATC | |
| | | AATGGGCGTGGATAGCGGTTTGACTCACGGGGATTTC | |
| | | CAAGTCTCCACCCCATTGACGTCAATGGGAGTTTGTT | |
| | | TTGGCACCAAAATCAACGGGACTTTCCAAAATGTCGT | |
| | | AACAACTCCGCCCCATTGACGCAAATGGGCGGTAGGC | |
| | | GTGTACGGTGGGAGGTCTATATAAGCAGAGCTC | |
| 16 | bgh terminator | CTGTGCCTTCTAGTTGCCAGCCATCTGTTGTTTGCCC | DNA |
| | | CTCCCCCGTGCCTTCCTTGACCCTGGAAGGTGCCACT | |
| | | CCCACTGTCCTTTCCTAATAAAATGAGGAAATTGCAT | |
| | | CGCATTGTCTGAGTAGGTGTCATTCTATTCTGGGGGG | |
| | | TGGGGTGGGGCAGGACAGCAAGGGGGAGGATTGGGAA | |
| | | GACAATAGCAGGCATGCTGGGGATGCGGTGGGCTCTA | |
| | | TGG | |
| 17 | RTPS1 V2271 | MCKSVTTDEWKKVFYEKMEEAKPADSWDLIIDPNLKH | aa |
| | | NVLSPGWKQYLELHASGRFHCSWCWHTWQSPYVVILF | |
| | | HMFLDRAQRAGSVRMRVFKQLCYECGTARLDESSMLE | |
| | | ENIEGLVDNLITSLREQCYGERGGQYRIHVASRQDNR | |
| | | RHRGEFCEACQEGIVHWKPSEKLLEEEATTYTFSRAP | |
| | | SPTKSQDQTGSGWNFCSIPWCLFWATVLLLIIYLQFS | |
| | | FRSSI | |
| 18 | RTP2 L220R | MCTSLTTCEWKKVFYEKMEVAKPADSWELIIDPNLKP | aa |
| | | SELAPGWKQYLEQHASGRFHCSWCWHTWQSAHVVILF | |
| | | HMFLDRAQRAGSVRMRVFKQLCYECGTARLDESSMLE | |
| | | ENIEGLVDNLITSLREQCYEEDGGQYRIHVASRPDSG | |
| | | PHRAEFCEACQEGIVHWKPSEKLLEEEVTTYTSEASK | |
| | | PRAQAGSGYNFLSLRWCLFWASLCLLVVYLQFSFRSP | |
| | | AFF | |
| 19 | hOR5A2 | MAVGRNNTIVTKFILLGLSDHPQMKIFLFMLFLGLYL | aa |
| | | LTLAWNLSLIALIKMDSHLHMPMYFFLSNLSFLDICY | |
| | | VSSTAPKMLSDIITEQKTISFVGCATQYFVFCGMGLT | |
| | | ECFLLAAMAYDRYAAICNPLLYTVLISHTLCLKMVVG | |
| | | AYVGGFLSSFIETYSVYQHDFCGPYMINHFFCDLPPV | |
| | | LALSCSDTFTSEVVTFIVSVVVGIVSVLVVLISYGYI | |
| | | VAAVVKISSATGRTKAFSTCASHLTAVTLFYGSGFFM | |
| | | YMRPSSSYSLNRDKVVSIFYALVIPVVNPIIYSFRNK | |
| | | EIKNAMRKAMERDPGISHGGPFIFMTLG | |
| 20 | hOR5A2-clone 3 | MAVGRNNTIVTKFILLGLSDHPQMKIFLFMLFLGLYL | aa |
| | | LTLAWNLSLIALIKMDSHLHMPMYFFLSNLSFLDICY | |
| | | VSSTAPKMLSDIITEQKTISFVGCATQYFVFCGMGLT | |
| | | ECFLLAAMAYDRYAAICNPLLYTVLISHTLCLKMVVG | |
| | | AYVGGFLSSFIETYSVYQHDFCGPYMINHFFCDLPPV | |
| | | LALSCSDTFTSEVVTFIVSVVVGIVSVLVVLISYGYI | |
| | | VAAVVKISSATGRTKAFSTCASHLTAVTLFYGSGFFM | |
| | | YMRPSSSYSLNRDKVVSIFYALVIPVVNPIIYSFRNK | |
| | | EIKNAMRKAMESWPRCCRSW | |
| 21 | primer | CAGAGATCTCGCGTTGACATTGATTATTGACTAG | DNA |
| 22 | primer | CTGGTCGACAGAAGCCATAGAGCCCAC | DNA |
| 23 | primer | CTGGTCGACCGCGTTGACATTGATTATTGACTAG | DNA |
| 24 | primer | GCGGCCAAGCTTAGACACTAGAG | DNA |
| 25 | primer | CGGTCATGGTGGCTTTACCAACAGTACC | DNA |
| 26 | primer | TGGTAAAGCCACCATGACCGAGTACAAGC | DNA |
| 27 | primer | CAGTCTAGATCAGGCACCGGGCTTG | DNA |
| 28 | primer | AACATTTCTCTGGCCTAACTGG | DNA |
| 29 | primer | ATTCCCGATGATGAGCACTTTC | DNA |
| 30 | primer | TACAGGAATTCATGGCTGTAGGAAGGAACAAC | DNA |
| 31 | primer | ACTGCGGCCGCTTACCATGAGCGACAACACCG | DNA |
| 32 | primer | ACAAGGACGACGACGATAAG | DNA |
| 33 | primer | GATGGCTGGCAACTAGAAGG | DNA |
| 34 | primer | ATTAAGGTACGGGAGGTATTGG | DNA |
| 35 | primer | AAGAGTGGGCTATATCGAACTG | DNA |
| 36-44 | M1-M9 | shown in FIG. 9A-9D | DNA |
| 45-53 | E1-E9 | shown in FIG. 10A-10D | DNA |
| 54-61 | V1-V9 | shown in FIG. 11A-11D | DNA |
| 62 | C-terminus | VVSIFYALVIPVVNPIIYSFRNKEIKNAMRKAMESWP | aa |
| | OR5A2 clone 3 | RCCRSW | |
| 63 | primer | GACAAAGGCATGGTGGCTTTACCAACAG | DNA |
| 64 | bsd | ATGCCTTTGTCTCAAGAAGAATCCACCCTCATTGAAA | DNA |
| | | GAGCAACGGCTACAATCAACAGCATCCCCATCTCTGA | |
| | | AGACTACAGCGTCGCCAGCGCAGCTCTCTCTAGCGAC | |
| | | GGCCGCATCTTCACTGGTGTCAATGTATATCATTTTA | |
| | | CTGGGGGACCTTGTGCAGAACTCGTGGTGCTGGGCAC | |
| | | TGCTGCTGCTGCGGCAGCTGGCAACCTGACTTGTATC | |
| | | GTCGCGATCGGAAATGAGAACAGGGGCATCTTGAGCC | |
| | | CCTGCGGACGGTGCCGACAGGTGCTTCTCGATCTGCA | |
| | | TCCTGGGATCAAAGCCATAGTGAAGGACAGTGATGGA | |
| | | CAGCCGACGGCAGTTGGGATTCGTGAATTGCTGCCCT | |
| | | CTGGTTATGTGTGGGAGGGCTAA | |
| 65 | bsd | MPLSQEESTLIERATATINSIPISEDYSVASAALSSD | aa |
| | | GRIFTGVNVYHFTGGPCAELVVLGTAAAAAAGNLTCI | |
| | | VAIGNENRGILSPCGRCRQVLLDLHPGIKAIVKDSDG | |
| | | QPTAVGIRELLPSGYVWEG | |
| 66 | primer | GTAAAGCCACCATGCCTTTGTCTCAAGAAGAATCC | DNA |
| 67 | primer | CCGACTCTAGATTAGCCCTCCCACACATAAC | DNA |
| 68 | primer | ATCAGGCCGGCCGCCCCGACTCTAGATTAGCCCTCC | DNA |
| 69 | C-terminus | VVSIFYALVIPVVNPIIYSFRNKEIKNAMRKAMERDP | aa |
| | hOR5A2 | GISHGGPFIFMTLG | |
| 70 | OR5AN1 | ATGACTGGGGGAGGAAATATTACAGAAATCACCTATT | DNA |
| | | TCATCCTGCTGGGATTCTCAGATTTTCCCAGGATCAT | |
| | | AAAAGTGCTCTTCACTATATTCCTGGTGATCTACATT | |
| | | ACATCTCTGGCCTGGAACCTCTCCCTCATTGTTTTAA | |
| | | TAAGGATGGATTCCCACCTCCATACACCCATGTATTT | |
| | | CTTCCTCAGTAACCTGTCCTTCATAGATGTCTGCTAT | |
| | | ATCAGCTCCACAGTCCCCAAGATGCTCTCCAACCTCT | |
| | | TACAGGAACAGCAAACTATCACTTTTGTTGGTTGTAT | |
| | | TATTCAGTACTTTATCTTTTCAACGATGGGACTGAGT | |
| | | GAGTCTTGTCTCATGACAGCCATGGCTTATGATCGTT | |
| | | ATGCTGCCATTTGTAACCCCCTGCTCTATTCATCCAT | |
| | | CATGTCACCCACCCTCTGTGTTTGGATGGTACTGGGA | |
| | | GCCTACATGACTGGCCTCACTGCTTCTTTATTCCAAA | |
| | | TTGGTGCTTTGCTTCAACTCCACTTCTGTGGGTCTAA | |
| | | TGTCATCAGACATTTCTTCTGTGACATGCCCCAACTG | |
| | | TTAATCTTGTCCTGTACTGACACTTTCTTTGTACAGG | |
| | | TCATGACTGCTATATTAACCATGTTCTTTGGGATAGC | |
| | | AAGTGCCCTAGTTATCATGATATCCTATGGCTATATT | |
| | | GGCATCTCCATCATGAAGATCACTTCAGCTAAAGGCA | |
| | | GGTCCAAGGCATTCAACACCTGTGCTTCTCATCTAAC | |
| | | AGCTGTTTCCCTCTTCTATACATCAGGAATCTTTGTC | |
| | | TATTTGAGTTCCAGCTCTGGAGGTTCTTCAAGCTTTG | |
| | | ACAGATTTGCATCTGTTTTCTACACTGTGGTCATTCC | |
| | | CATGTTAAATCCCTTGATTTACAGTTTGAGGAACAAA | |
| | | GAAATTAAAGATGCCTTAAAGAGGTTGCAAAAGAGAA | |
| | | AGTGCTGCTGA | |
| 71 | OR10G7 | ATGTCCAACGCCTCCCTACTGACAGCGTTCATCCTCA | DNA |
| | | CGGGCCTTCCCCATGCCCCAGGGCTGGACGCCCCCCT | |
| | | CTTTGGAATCTTCCTGGTGGTTTACGTGCTCACTGTG | |
| | | CTGGGGAACCTCCTCATCCTGCTGGTGATCAGGGTGG | |
| | | ATTCTCACCTCCACACCCCCATGTACTACTTCCTCAC | |
| | | CAACCTGTCCTTCATTGACATGTGGTTCTCCACTGTC | |
| | | ACGGTGCCCAAAATGCTGATGACCTTGGTGTCCCCAA | |
| | | GCGGCAGGACTATCTCCTTCCACAGCTGCGTGGCTCA | |
| | | GCTCTATTTTTTCCACTTCCTGGGGAGCACCGAGTGT | |
| | | TTCCTCTACACAGTCATGTCCTATGATCGCTACCTGG | |
| | | CCATCAGTTACCCGCTCAGGTACACCAACATGATGAC | |
| | | TGGGCGCTCGTGTGCCCTCCTGGCCACCGGCACTTGG | |
| | | CTCAGTGGCTCTCTGCACTCTGCTGTCCAGACCATAT | |
| | | TGACTTTCCATTTGCCCTACTGTGGACCCAACCAGAT | |
| | | CCAGCACTACTTCTGTGACGCACCGCCCATCCTGAAA | |
| | | CTGGCCTGTGCAGACACCTCAGCCAACGAGATGGTCA | |
| | | TCTTTGTGAATATTGGGCTAGTGGCCTCGGGCTGCTT | |
| | | TGTCCTGATAGTGCTGTCCTATGTGTCCATCGTCTGT | |
| | | TCCATCCTGCGGATCCGCACCTCAGAGGGGAGGCACA | |
| | | GAGCCTTTCAGACCTGTGCCTCCCACTGTATCGTGGT | |
| | | CCTTTGCTTCTTTGGCCCTGGTCTTTTCATTTACCTG | |
| | | AGGCCAGGCTCCAGGGACGCCTTGCATGGGGTTGTGG | |
| | | CCGTTTTCTACACCACGCTGACTCCTCTTTTCAACCC | |
| | | TGTTGTGTACACCCTGAGAAACAAGGAGGTAAAGAAA | |
| | | GCTCTGTTGAAGCTGAAAAATGGGTCAGTATTTGCTC | |
| | | AGGGTGAATAG | |
| 72 | OR10G4 | ATGTCCAACGCCAGCCTCGTGACAGCATTCATCCTCA | DNA |
| | | CAGGCCTTCCCCATGCCCCAGGGCTGGACGCCCTCCT | |
| | | CTTTGGAATCTTCCTGGTGGTTTACGTGCTCACTGTG | |
| | | CTGGGGAACCTCCTCATCCTGCTGGTGATCAGGGTGG | |
| | | ATTCTCACCTCCACACCCCCATGTACTACTTCCTCAC | |
| | | CAACCTGTCCTTCATTGACATGTGGTTCTCCACTGTC | |
| | | ACGGTGCCCAAAATGCTGATGACCTTGGTGTCCCCAA | |
| | | GCGGCAGGGCTATCTCCTTCCACAGCTGCGTGGCTCA | |
| | | GCTCTATTTTTTCCACTTCCTGGGGAGCACCGAGTGT | |
| | | TTCCTCTACACAGTCATGTCCTATGATCGCTACTTGG | |
| | | CCATCAGTTACCCGCTCAGGTACACCAGCATGATGAG | |
| | | TGGGAGCAGGTGTGCCCTCCTGGCCACCGGCACTTGG | |
| | | CTCAGTGGCTCTCTGCACTCTGCTGTCCAGACCATAT | |
| | | TGACTTTCCATTTGCCCTACTGTGGACCCAACCAGAT | |
| | | CCAGCACTACTTCTGTGACGCACCGCCCATCCTGAAA | |
| | | CTGGCCTGTGCAGACACCTCAGCCAACGTGATGGTCA | |
| | | TCTTTGTGGACATTGGGATAGTGGCCTCAGGCTGCTT | |
| | | TGTCCTGATAGTGCTGTCCTATGTGTCCATCGTCTGT | |
| | | TCCATCCTGCGCATCCGCACCTCAGATGGGAGGCGCA | |
| | | GAGCCTTTCAGACCTGTGCCTCCCACTGTATTGTGGT | |
| | | CCTTTGCTTCTTTGTTCCCTGTGTTGTCATTTATCTG | |
| | | AGGCCAGGCTCCATGGATGCCATGGATGGAGTTGTGG | |
| | | CCATTTTCTACACTGTGCTGACGCCCCTTCTCAACCC | |
| | | TGTTGTGTACACCCTGAGAAACAAGGAGGTGAAGAAA | |
| | | GCTGTGTTGAAACTTAGAGACAAAGTAGCACATCCTC | |
| | | AGAGGAAATAA | |

### General Information

Unless stated otherwise, all technical and scientific terms used herein have the same meaning as customarily and ordinarily understood by a person of ordinary skill in the art to which this invention belongs, and read in view of this disclosure.

### Sequence Identity

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60%, 70%, 80%, 90%, 95% or 99% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 60%, 70%, 80%, 90%, 95% or 99% amino acid identity or similarity with an amino acid sequence encoded by a nucleotide sequence SEQ ID NO: X.

Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide represented by an amino acid sequence comprising a sequence that has at least 60%, 70%, 80%, 90%, 95% or 99% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

Each non-coding nucleotide sequence (i.e. of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: A. In a preferred embodiment, such non-coding nucleotide sequence such as a promoter exhibits or exerts at least an activity of such a non-coding nucleotide sequence such as an activity of a promoter as known to a person of skill in the art.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated by reference herein in its entirety.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference in its entirety).

Alternatively, percentage similarity or identity may be determined by searching against public databases, using algorithms such as FASTA, BLAST, etc. Thus, the nucleic acid and protein sequences of some embodiments of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference in its entirety. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402, incorporated herein by reference in its entirety. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, |
| | M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, |
| | N, Q, R, S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; IIe to Leu or Val; Leu to IIe or Val; Lys to Arg; Gln or Glu; Met to Leu or IIe; Phe to Met, Leu or Tyr; Ser to Thr; Thr to Ser; Trp to Tyr; Tyr to Trp or Phe; and, Val to IIe or Leu.

### Gene or Coding Nucleotide Sequence

The term “gene” refers to a DNA fragment comprising a region (transcribed region), which is transcribed into an RNA molecule (e.g. an mRNA) in a cell, operably linked to suitable regulatory regions (e.g. a promoter). Coding nucleotide sequences may comprise sequences that are native to the cell, sequences that naturally do not occur in the cell and it may comprise combinations of both.

### Proteins and Amino Acids

The terms “protein” or “peptide” or “polypeptide” or “amino acid sequence” are used interchangeably and refer to molecules consisting of a chain of amino acids, without reference to a specific mode of action, size, 3-dimensional structure or origin. In amino acid sequences as described herein, amino acids or “residues” are denoted by three-letter symbols. These three-letter symbols as well as the corresponding one-letter symbols are well known to a person of skill in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (IIe) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid.

### General Terms

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristics of the invention. In addition, the verb “to consist” may be replaced by “to consist essentially of” meaning that a method or use as described herein may comprise additional step(s) than the ones specifically identified, said additional step(s) not altering the unique characteristic of the invention. In addition, the verb “to consist” may be replaced by “to consist essentially of” meaning that a nucleotide or amino acid sequence as described herein may comprise additional nucleotides or amino acids than the ones specifically identified, said additional nucleotides or amino acids not altering the unique characteristics of the invention.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The word “about” or “approximately” when used in association with a numerical value (e.g. about 10) preferably means that the value may be the given value (of 10) more or less 1% of the value.

As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1. HEK293T cells with stable insertion of pGL4.29-CRE-Puro-Hygro-OR containing the receptor OR10G7 exposed to 11 concentrations of puromycin and co-treated with different olfactory ligands. Cells expressing OR10G7 have an increased resistance to puromycin in presence of the cognate ligands eugenol, isoeugenol, vanitrope and ultravanil, while the odorant musk ketone, which does not bind to OR10G7, does not increase resistance over solvent control.

FIG. 2. HEK293T cells with stable insertion of pGL4.29-CRE-Puro-Hygro-OR containing the receptor OR5AN1 exposed to 11 concentrations of puromycin and co-treated with different olfactory ligands. Cells expressing OR5AN1 have an increased resistance to puromycin in presence of musk ketone, while the ligands eugenol, isoeugenol and ultravanil which do not bind to OR5AN1 do not increase resistance over solvent control.

FIG. 3. HEK293T cells with stable insertion of pGL4.29-CRE-Puro-Hygro-OR containing the receptor OR10G9 exposed to 11 concentrations of puromycin and co-treated with different olfactory ligands. Cells expressing OR10G9 have an increased resistance to puromycin in presence of the cognate ligands vanitrope and ultravanil, while no increased resistance to musk ketone, eugenol and isoeugenol, which do not bind to this receptor at the given concentration, is observed over solvent control.

FIG. 4. Resistance of an isolated clone (“Clone 3”) containing the receptor OR5A2 in a pGL4.29-CRE-Puro-Hygro-OR construct as described in Example 1, exposed to 11 concentrations of puromycin and co-treated with different olfactory ligands. The isolated clone functionally expressing OR5A2 has an increased resistance to puromycin in the presence of the cognate musk odorant ligands musk ambrette and serenolide, while no increased resistance in the presence of eugenol, which does not bind to this receptor, was observed.

FIG. 5A-5B. Odorant induced luciferase signal in HEK 293T cells containing CRE-inducible luciferase and (FIG. 5A) containing a variant of RTP1S and RTP2 and OR5A2, but which had not been selected for ligand-induced puromycin resistance and (FIG. 5B) in cells of “clone 3” which is stably transfected with the same three genes and which has been selected for functional expression from a pool of 2.5 million cells by ligand-induced puromycin resistance as described herein. “Clone 3” contains an acquired mutation conferring functional expression of OR5A2 which could be selected from a pool of 2.5 Mio cells by the specific applied selection procedure.

FIG. 6. Amino acid sequence alignment of OR5A2 wildtype (SEQ ID NO: 69) with the mutant form in “clone 3” (SEQ ID NO: 62). In dark grey shading the seventh transmembrane domain is shown, while in light dark shading the wildtype C-terminus is depicted. The amino acids in bold in the boxed sequence were acquired in “clone 3” by a recombination event, and the corresponding cell containing this mutation was selected based on the inventive selection procedure.

FIG. 7. Luciferase induction by musk ligands in HEK293T cells expressing functional variants of the human RTP1S V2271 (SEQ ID NO: 17) and RTP2 L220R (SEQ ID NO: 18) and transfected with CRE-inducible luciferase and with the mutant form of OR5A2 shown in FIG. 6.

FIG. 8. HEK293T cells with stable insertion of pGL4.29-CRE-BLAST-OR containing the receptor OR5AN1 exposed to 11 concentrations of blasticidin and co-treated with different olfactory ligands. Cells expressing OR5AN1 have an increased resistance to blasticidin in presence of the ligand musk ketone, while the ligand ethylvanillin, which does not bind to OR5AN1, does not increase resistance over solvent control.

FIG. 9A-9D. Clones selected with musk ketone (SEQ ID NO: 36-44; M1 SEQ ID NO: 36, M2 SEQ ID NO: 37, M3 SEQ ID NO: 38, M4 SEQ ID NO: 39, M5 SEQ ID NO: 40, M6 SEQ ID NO: 41, M7 SEQ ID NO: 42, M8 SEQ ID NO: 43, M9 SEQ ID NO: 44) alignment with OR5AN1 (SEQ ID NO: 70).

FIG. 10A-10D. Clones selected with eugenol (SEQ ID NO: 45-53; E1 SEQ ID NO: 45, E2 SEQ ID NO: 46, E3 SEQ ID NO: 47, E4 SEQ ID NO: 48, E5 SEQ ID NO: 49, E6 SEQ ID NO: 50, E7 SEQ ID NO: 51, E8 SEQ ID NO: 52, E9 SEQ ID NO: 53) alignment with OR10G7 (SEQ ID NO: 71).

FIG. 11A-11D. Clones selected with vanitrope (SEQ ID NO: 54-61; V1 SEQ ID NO: 54, V2 SEQ ID NO: 55, V3 SEQ ID NO: 56, V5 SEQ ID NO: 57, V6 SEQ ID NO: 58, V7 SEQ ID NO: 59, V8 SEQ ID NO: 60, V9 SEQ ID NO: 61) alignment with OR10G4 (SEQ ID NO: 72).

### EXAMPLES

### Example 1. Generation of a Vector to Select for OR-Ligand Induced Puromycin Resistance and Generation of Stable Cell Lines Comprising Said Vector

To generate a vector allowing positive selection of cell clones with a functional expression of a specific OR gene, the puromycin resistance gene was placed downstream of SEQ ID NO: 4. The pGL4.29 plasmid (Promega, WI, USA) was digested with HindIII/XbaI (partial) to remove part of the comprised CRE and the complete luciferase coding sequence. The removed CRE sequence and the puromycin N-acetyltransferase (PAC, SEQ ID NO: 7) encoding sequence were amplified by PCR and joined by fusion PCR using primers: GCGGCCAAGCTTAGACACTAGAG (SEQ ID NO: 24), CGGTCATGGTGGCTTTACCAACAGTACC (SEQ ID NO: 25), TGGTAAAGCCACCATGACCGAGTACAAGC (SEQ ID NO: 26), and CAGTCTAGATCAGGCACCGGGCTTG (SEQ ID NO: 27). The resulting DNA fragment was digested with HindIII/XbaI and ligated into pGL4.29 to generate pGL4.29-CRE-Puro.

Construction of pGL4.29-CRE-Puro-OR

To create a plasmid harboring the PAC under control of CREs and an OR expression cassette, an OR coding sequence was PCR-amplified from pcDNA3.1(+) (Invitrogen, MA, USA) together with the CMV promoter sequence (SEQ ID NO: 15), a nucleotide sequence encoding a signal peptide (mmLucy-FLAG-rho, SEQ ID NO: 13) and the bgh terminator sequence (SEQ ID NO: 16) using the following primers: CAGAGATCTCGCGTTGACATTGATTATTGACTAG (SEQ ID NO: 21) and CTGGTCGACAGAAGCCATAGAGCCCAC (SEQ ID NO: 22). The PCR product was digested with BgIII/SaII and used to replace the BamHI/SaII fragment of pGL4.29-CRE-Puro. This plasmid thus contains a constitutively expressed OR gene and a CRE-inducible puromycin resistance.

Construction of pGL4.29-CRE-Puro-Hygro-OR

To create a plasmid harboring a constitutive hygromycin resistance gene (SEQ ID NO: 8), the PAC under control of CREs and an OR expression cassette, an OR coding sequence was PCR-amplified from pcDNA3.1(+) together with the CMV promoter sequence (SEQ ID NO 15), a nucleotide sequence encoding a signal peptide (mmLucy-FLAG-rho, SEQ ID NO: 13) and the BGH terminator sequence (SEQ ID NO: 16) using the following primers: CTGGTCGACCGCGTTGACATTGATTATTGACTAG (SEQ ID NO: 23) and CTGGTCGACAGAAGCCATAGAGCCCAC (SEQ ID NO: 22). The PCR product was digested with SaII and inserted into SaII-restricted pGL4.29-CRE-Puro. This plasmid thus contains a constitutively expressed OR gene, a constitutive Hygromycin resistance gene and a CRE-inducible Puromycin resistance.

10 μg of the resulting plasmid pGL4.29-CRE-Puro-Hygro-OR was then linearized by digestion in 150 μl with PvuI. The linearized plasmid was purified and 7.6 μg was diluted in 0.5 ml OptiMEM medium (Gibco™, ThermoFisher Scientific, MA, USA) containing 15 μl P3000 reagent (Invitrogen). In parallel 11.5 μl Lipofectamine 3000 (Invitrogen) was diluted in 0.5 ml OptiMEM medium and after 5 min pre-incubation, the two mixtures were combined to prepare the transfection mixture which was incubated for further 25 min.

Expression of the OR genes was in general performed in HEK293T cells which had been stably transfected with functional variants of the human RTP1S (V2271, SEQ ID NO: 17) and RTP2 (L220R, SEQ ID NO: 18). These cells were grown in 10 cm petri-dishes at 37° C. in presence of 5% CO₂ to sub-confluence. Growth medium was replaced with 10 ml DMEM containing 9% FBS and then the pre-incubated transfection mixture was added to the cells which were then incubated for 24 h at 37° C. in presence of 5% CO₂ to allow for DNA uptake and chromosomal insertion to take place. Cells were harvested and re-suspended in DMEM containing 9% FBS and a mixture of penicillin and streptomycin (ThermoFisher Scientific) and they were then seeded into 96-well plates (100 ul/well) at a density of 50 cells/well. 24 h after cell seeding, selection pressure was applied to select for cells with a stable insertion of the vector into the chromosome. In order to select for chromosomal insertion, hygromycin at a final concentration of 100 μg/ml was added to the cells, and the medium was replaced twice per week with fresh medium containing the same amount of hygromycin. Wells with single, isolated surviving clones were marked and these clones were harvested and expanded as stable cell lines containing the pGL4.29-CRE-Puro-Hygro-OR construct.

### Example 2. Ligand Induced OR-Dependent Resistance of Selected Cell Clones to Puromycin

Stable clones selected with hygromycin containing a stable insertion of the pGL4.29-CRE-Puro-Hygro-OR construct described in Example 1 with either one of the receptors OR10G7, OR10G9 or OR5AN1 (NCBI Genbank Gene ID 390265, 219870 and 390195) were plated at a density of 3000 cells/per well in 200 μl Dubelcco's Modified Eagle medium (DMEM) in polyethyleneimide-coated, white 96 well microtiter plates with a transparent bottom. After cells were allowed to adhere for 24 h, they were exposed to different odorants (musk ketone at 10 μM, eugenol or isoeugenol at 1 μM, ultravanil or vanitrope at 100 μM). 7 h after odorant addition, 100 μl of the medium was removed and replaced with fresh medium with the same odorant concentrations and, in addition, puromycin to reach a final concentration of 0.015-16 μg/ml in 11 twofold dilution steps (day 1 of selection). On day 2 and 3, 100 μl of the medium was again removed and replaced with fresh medium with the same concentration of odorant and puromycin. On day 4 of the selection, medium from each well was removed completely, 100 ul PrestoBlue® cell viability reagent (ThermoFisher Scientific, Catalog No: 14200-083) diluted in phosphate buffered saline was added to each well and cells were incubated at 37° C. until a color change was visible in the wells containing no puromycin. The fluorescence was determined at 560 excitation and 590 nm emission to assess relative cell metabolic activity, which is the net result of cell growth and cell killing in presence of the different puromycin concentrations. The result was expressed as % viability relative to controls with no cells (0%) and cells treated with olfactory ligand only (100%). Since cells were actively growing in this experiment a partial reduction in viability does not indicate cytotoxicity but can indicate (partial) cell stasis, while 0% viability indicates complete cytotoxicity.

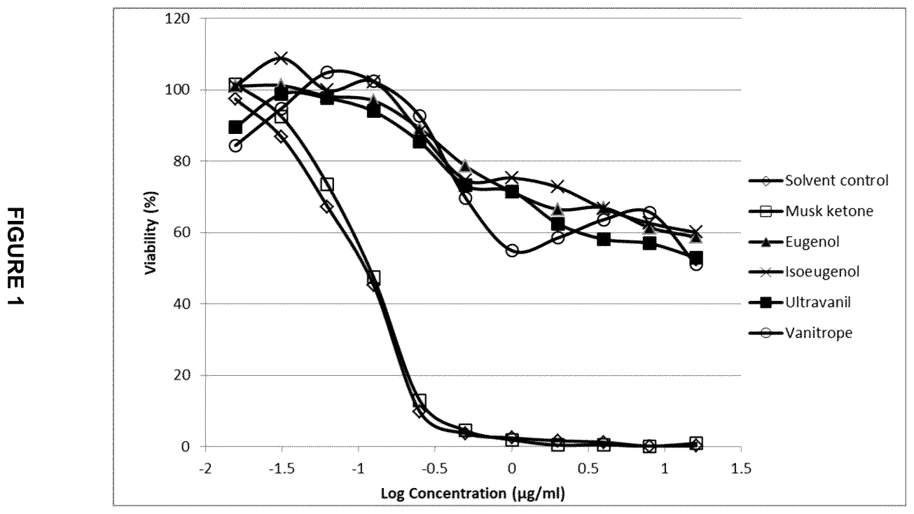

FIG. 1 shows the results for cells transformed with a vector as described in Example 1 and containing OR10G7. If cells were selected in presence of the odorant musk ketone, which is not a ligand for OR10G7, no induced puromycin resistance over solvent control was observed. However, with the ligands eugenol, isoeugenol, ultravanil and vanitrope, which bind to OR10G7, a strongly enhanced puromycin resistance was observed, and viability was only partly reduced by puromycin.

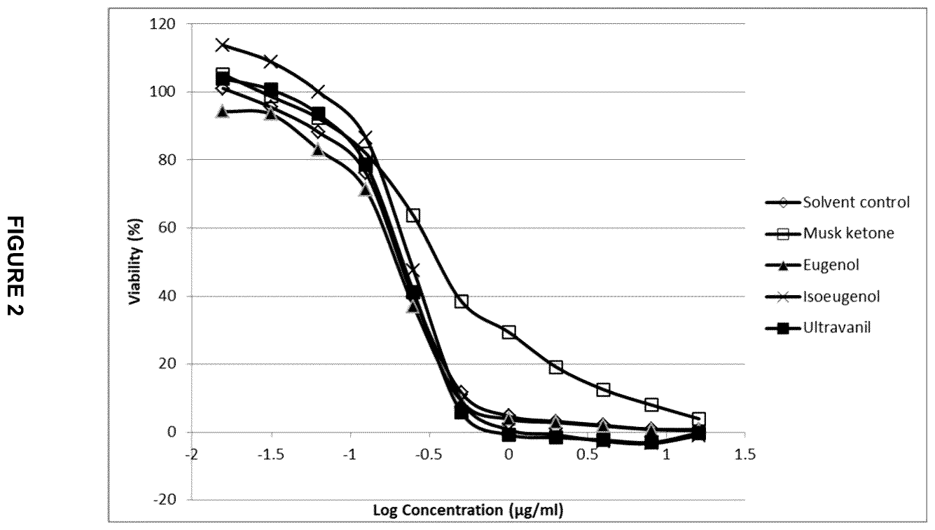

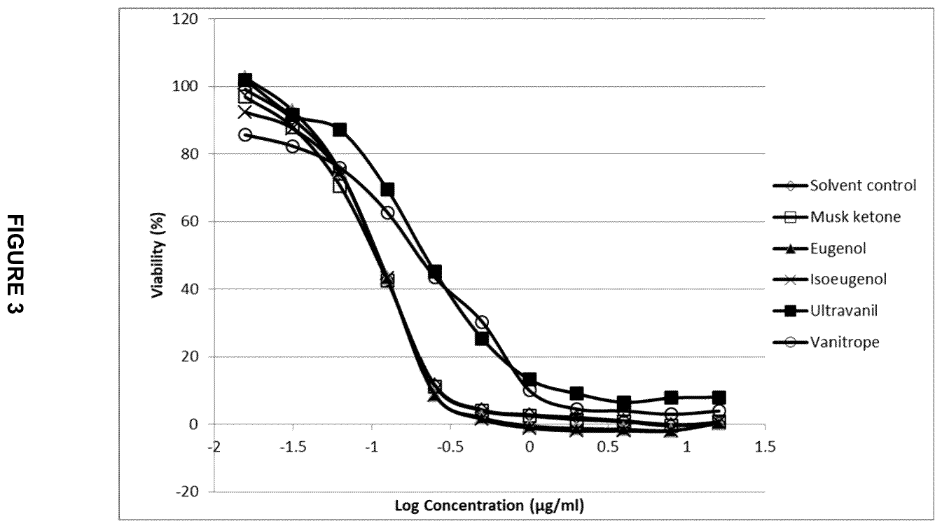

FIG. 2 shows the same experiment performed with OR5AN1, which is known to respond to musk compounds such as musk ketone. In this case, enhanced puromycin resistance and only partial reduction of viable cells was observed in presence of the musk ligand, while in presence of eugenol, isoeugenol and ultravanil, which are not ligands to this receptor, the resistance was equal to the solvent control. Similarly, as shown in FIG. 3, for cells harboring the vector with OR10G9, a selectively enhanced resistance for the cognate ligands ultravanil and vanitrope was observed, while no increased resistance to musk ketone, eugenol and isoeugenol, which do not bind to this receptor at the given concentration, was observed.

These results show that it is possible to selectively induce resistance to a selectable marker (in this case puromycin) by the activation of the cyclic AMP pathway acting on the CRE element with the functional expression of an olfactory receptor and addition of its cognate ligand.

Example 3. Selection of a Cell Clone with Functional Expression of a Difficult-to-Express OR

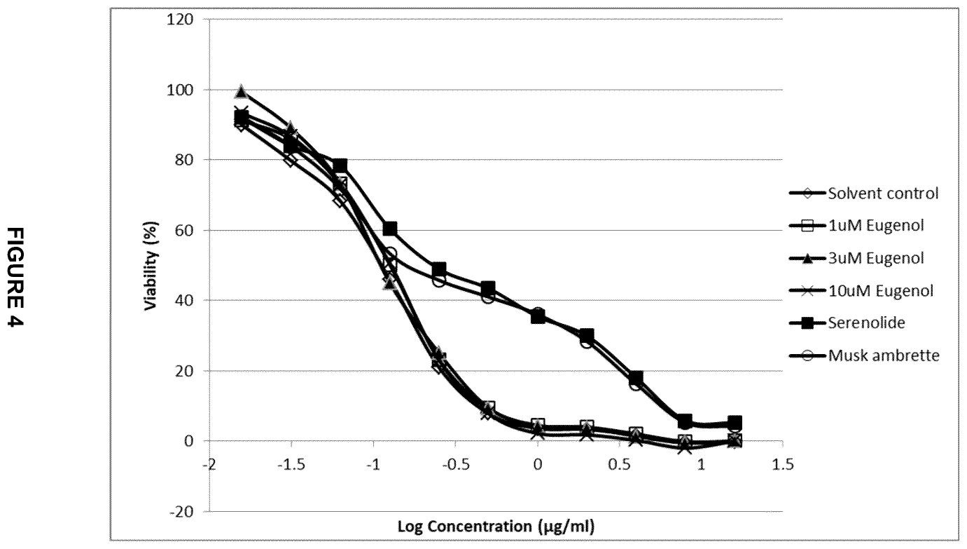

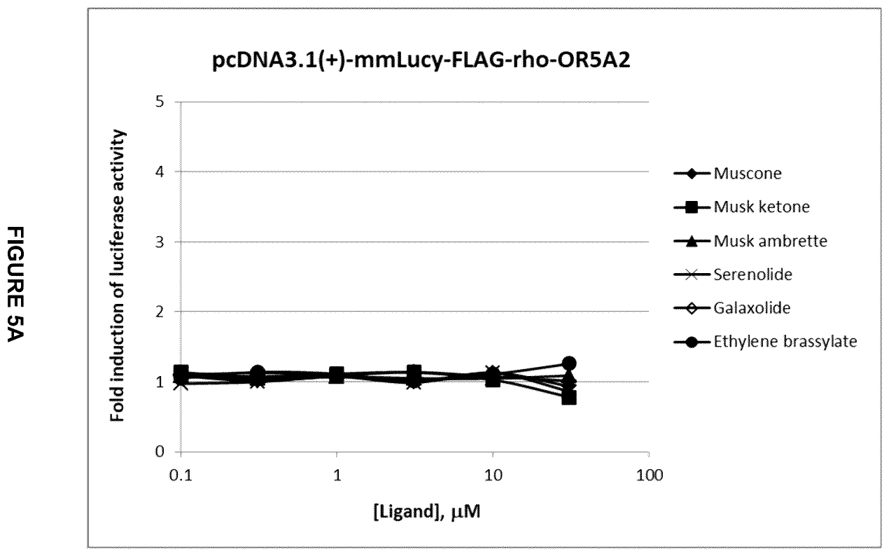

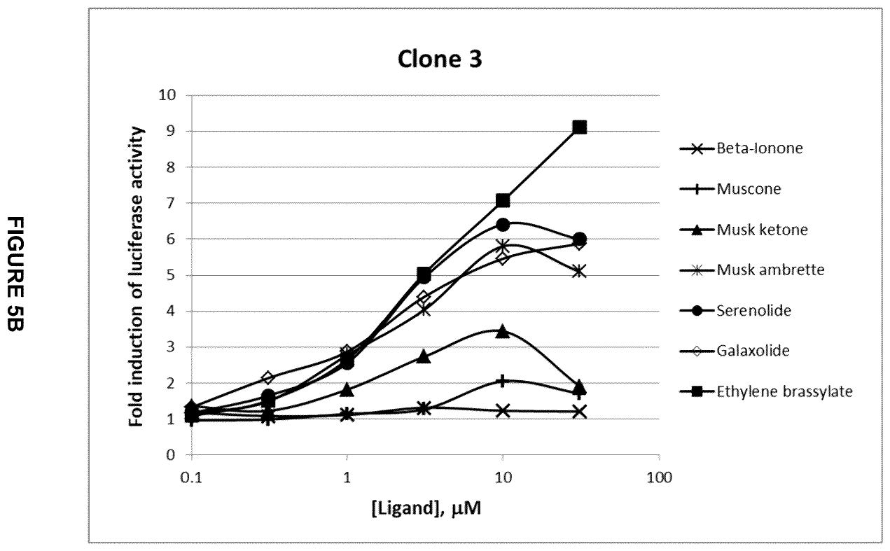

A pGL4.29-CRE-Puro-OR vector containing the receptor OR5A2 (NCBI Genbank ID: 219981) was generated as described in Example 1. The wild-type sequence of human OR5A2 codes for a difficult-to-express receptor and cannot be expressed in cells expressing the chaperone RTP1S and RTP2. It requires unknown accessory factors for functional expression. OR5A2 was postulated to be a musk receptor in WO2019110630A1, and genetic variants in this receptor were shown to correlate to the sensitivity for the musk ligand galaxolide in Trimmer et al. (2019) PNAS 116 (19): 9475-9480. These data collectively indicate that OR5A2 is a candidate musk receptor. This construct was transfected into 2.5 million HEK293T cells which had been stably transfected with variants of RTP1S and RTP2 as described in example 1. 24 h after transfection, cells were re-suspended in 240 ml DMEM with 9% FBS and distributed into 12-well plates (1 ml/well). Instead of selecting with hygromycin as in Example 1, a direct selection according the procedure of this invention was performed as follows: After 24 h incubation, to allow for cell adherence, the ligand musk ambrette was added to a final concentration of 30 μM to stimulate OR5A2 and hence start cAMP production. 7 h later, puromycin was added to a final level of 3 μg/ml. Cells were then continuously exposed to this concentration of ligand and puromycin by repeatedly exchanging the incubation medium, and single clones surviving this treatment were isolated and tested for ligand-dependent resistance and functional expression of OR5A2. Out of 2.5 million cells used for the experiment, only one clone (“clone 3”) was isolated which was able to survive puromycin treatment in presence of the ligand musk ambrette but not in its absence. This clone was first characterized by evaluation of its selective, OR-ligand-induced resistance as described in Example 2. As shown in FIG. 4, this clone had acquired a ligand-induced puromycin resistance indicating that it can functionally express the receptor OR5A2 and generate cAMP upon ligand addition. This clone was further analyzed for functional expression: It was grown in 96-well plates at a density of 10000 cells/well and transiently transfected using lipofectamine 2000 as described in Example 1 with the plasmid pGL4.29 (Promega) harbouring the CRE-inducible luciferase. 24 h after transfection, cells were stimulated with different ligands and 4.5 h later the luciferase signal, which is induced based on OR-dependent cAMP production, was measured. FIG. 5a shows results for HEK293T cells that had been stably transfected with variants of RTP1S and RTP2 (SEQ ID NO: 17 and 18) and with the receptor OR5A2, but which had not undergone a selection for functional expression according this invention. No ligand-induced luciferase signal was detected indicating that this receptor is not functional, even in cells expressing RTP1S and RTP2. In FIG. 5b, results for the OR5A2 harboring “clone 3”, which was derived from the selection procedure as described above is shown. This clone showed a strong ligand-induced luciferase signal, confirming that by this ligand-based selection procedure, a rare mutation (among 2.5 millions of cells) was selected which allows functional expression of this difficult-to-express receptor.

Example 4. Forward Gain-of-Function Mutagenesis with a Retrovirus Followed by Selection of a Cell Clone with Functional Expression of a Difficult-to-Express OR

A pGL4.29-CRE-Puro-Hygro-OR vector containing the receptor OR5A2 (NCBI Genbank Gene ID: 219981) was generated as described in Example 1. HEK293T cells which had been stably transfected with variants of RTP1S and RTP2 as described in Example 1 were transfected with this new construct and stable clones were selected in presence of hygromycin as described in Example 1. Selected clones were tested for puromycin resistance, and a single clone which is sensitive to 0.5 μg/ml of puromycin was selected and expanded. This provided for a uniform pool of cells with an identical insertion site of the pGL4.29-CRE-Puro-Hygro-OR vector and a high susceptibility for puromycin.

Retrovirus particles were produced by transfection of HEK293T cells with the vectors (i) pCCLsin.PPT.SFFV or pCCLsin.PPT.eGFP.sPRE.3′LTRsenseSFFV, (ii) pK-Rev, (iii) pMD2-VSV-G and (iv) pMDLg-pRRE. These vectors allow the cells to produce infective but non-replicating virus particles containing a strong SFFV promotor in the viral genome/vector as described by Montini et al. (2009) J Clin Invest 119:964-75, and Ranzani et al. (2014) Mol Ther 22 (12): 2056-2068. 24-36 hours post-transfection, the cell supernatant containing the viral particles was harvested.

The highly puromycin sensitive stable clone described above containing the pGL4.29-CRE-Puro-Hygro-OR vector was seeded in 10-cm petri dishes at 3×10⁵ cells per plate. The cells were incubated for 24 hours to allow for adherence and were then infected with a dilution of the viral particles (Multiplicity of infection, MOI=8) and incubated for 3 days allowing for infection and integration of the viral vector into the chromosomes. This led to random mutagenesis events by insertion of the strong promotor SFFV at different sites of the chromosomes in individual cells. Then, a direct selection procedure was performed as follows: The ligand musk ambrette was added to a final concentration of 30 μM to stimulate OR5A2 and hence start cAMP production. 7 h later, puromycin was added to a final level of 4 μg/ml. Cells were then continuously exposed to this concentration of ligand and puromycin by repeatedly exchanging the incubation medium, and single clones surviving this treatment were isolated and tested for ligand-dependent resistance and functional expression of OR5A2 as described in Example 3.

Example 5. Forward Gain-of-Function Mutagenesis Using CRISPRa Followed by Selection of a Cell Clone with Functional Expression of a Difficult-to-Express OR

The highly puromycin sensitive stable clone of Example 4 containing the pGL4.29-CRE-Puro-Hygro-OR vector containing the receptor OR5A2 is transduced with a lentivirus to express dCas9-VPH gene, which codes for a “catalytic dead” Cas9 fused to the VP64, p65, and HSF1 transactivation domains (Plasmid pRDVCRB-RSV-dCas9-VPH-2A-Blast which can be obtained from Cellecta Inc. CA, USA). Stable clones are selected in the presence of blasticidin. A stable clone is then seeded in 10-cm petri dishes at 3×10⁵ cells per plate. The cells are incubated for 24 hours to allow for adherence and are then infected with the viral particles obtained from Cellecta Inc. (Catalog nummer KADHGW-105K-V9; https://cellecta.com/collections/crispra-and-crispri-lentiviral-sgrna-libraries). These virus particles encode for a genome-wide library of guide-RNA (sgRNA). Cells are incubated for 3 days allowing for infection and integration of the viral vector into the chromosomes. The dCas9-VPH protein then leads to activation of the gene adjacent of the binding site of the sgRNA. Then a direct selection according the procedure of this invention is performed as follows: The ligand musk ambrette is added to a final concentration of 30 μM to stimulate OR5A2 and hence start cAMP production. 7 h later, puromycin is added to a final level of 4 μg/ml. Cells are then continuously exposed to this concentration of ligand and puromycin by repeatedly exchanging the incubation medium. Single clones surviving this treatment are isolated and tested for ligand-dependent resistance and functional expression of OR5A2 as described in Example 3.

### Example 6. Characterization of the Mutation in Clone 3

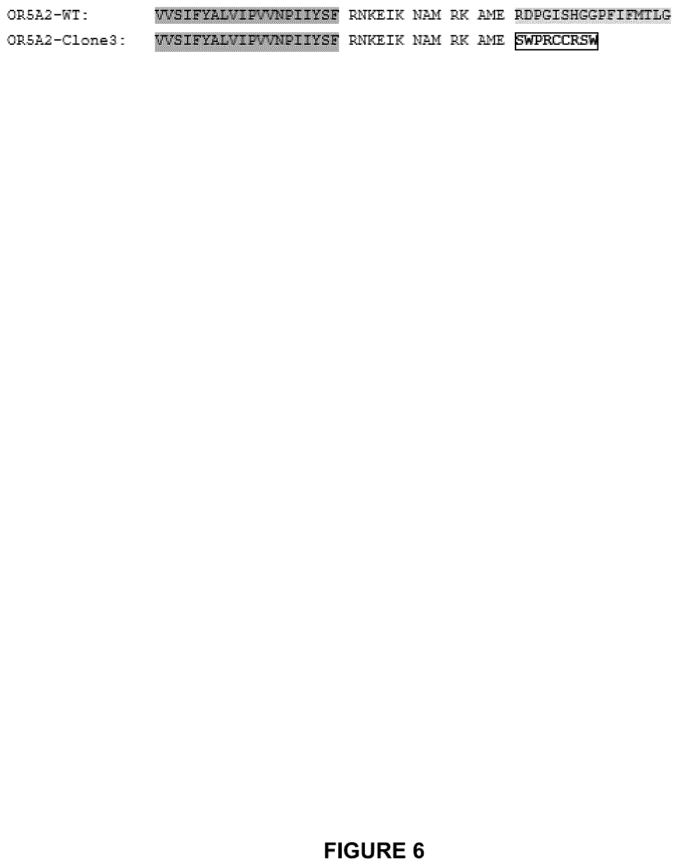

To identify the integration sites of the transfected plasmids, chromosomal DNA from “clone 3” as described in Example 3 was extracted according to standard procedures. The DNA was separately digested with the following restriction enzymes: BamHI, BgIII, HindIII, NcoI, NdeI, NheI, SpeI. The separate digestions were each subjected to a ligation reaction to generate circular DNA fragments and then subjected to nested PCR using the following primer pairs: 1st PCR: 5′-ATTAAGGTACGGGAGGTATTGG-3′ (SEQ ID NO: 34) and 5′-AAGAGTGGGCTATATCGAACTG-3′ (SEQ ID NO: 35); nested PCR: 5′-AACATTTCTCTGGCCTAACTGG-3′ (SEQ ID NO: 28) and 5′-ATTCCCGATGATGAGCACTTTC-3′ (SEQ ID NO: 29). The sequences of the resulting PCR products were determined with Sanger sequencing. The result indicated that in clone 3, the OR5A2 gene originating from the inserted plasmid had acquired a mutation of its C-terminus by a recombination event. The alignment in FIG. 6 shows the wildtype amino acid sequence of OR5A2 (SEQ ID NO: 69) starting with the last transmembrane region until the C-terminus and the mutated sequence of OR5A2 amplified from “clone 3” (SEQ ID NO: 62). The complete sequence of OR5A2 from “clone 3” is represented by SEQ ID NO: 20 and the C-terminus end is represented by SEQ ID NO: 62.

To verify, that the identified mutation indeed confers to OR5A2 the ability to be functionally expressed in HEK293T cells, the OR5A2 variant was PCR-amplified from genomic DNA of clone 3 using primers 5′-TACAGGAATTCATGGCTGTAGGAAGGAACAAC-3′ (SEQ ID NO: 30) and 5′-ACTGCGGCCGCTTACCATGAGCGACAACACCG-3′ (SEQ ID NO: 31) and cloned into the expression vector pcDNA3.1(+) downstream of the signal peptide (SEQ ID NO: 14).

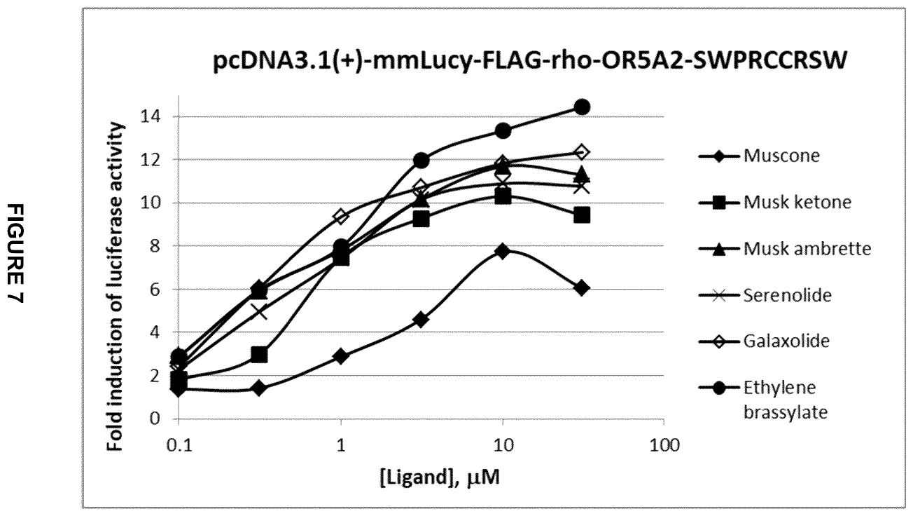

The resulting plasmid with OR5A2 with the same introduced C-terminal mutation as shown in FIG. 6 was then used for transient transfection of HEK293T cells which had been stably transfected with functional variants of the human RTP1S (V2271, SEQ ID NO: 17) and RTP2 (L220R, SEQ ID NO: 18). Functional expression and response to musk ligands could be verified by co-transfection of CRE-inducible luciferase and stimulation with different musk ligands, as shown in FIG. 7. This proves that the stable mutation of the C-terminus is sufficient to confer to OR5A2 the ability to be functionally expressed.

Example 7. Selection of a Cell Expressing a Specific Cognate Receptor from a Pool of Receptor-Expressing Cells by Selection with the Target Ligand

For many ligands the cognate OR receptors are not known (“orphan receptors”), or it is unknown among a pool of receptors, which one has the best affinity for a ligand of interest, e.g. a perfumery note of particular interest. Thus, the ligand-induced selection pressure can be used to enrich a specific OR-expressing cell from a pool of OR-expressing cells in the presence of a ligand of interest. HEK293T cells stably expressing RTP1S and RTP2 were seeded into 7 wells of 6-well plates at 330,000 cells/well.

The next day, the cells of each well were transfected with 2 μg of a PvuI-linearized pGL4.29-CRE-Puro-Hygro-OR construct harboring one of the receptors OR5A1 (NCBI Genbank Gene ID: 219982), OR5A2 (NCBI Genbank Gene ID: 219981), OR5AN1 NCBI Genbank Gene ID: 390195, SEQ ID NO: 70), OR6Y1 (NCBI Genbank Gene ID: 391112), OR10G4 (NCBI Genbank Gene ID: 390264, SEQ ID NO: 72), OR10G7 (NCBI Genbank Gene ID: 390265, SEQ ID NO: 71), or OR10G9 (NCBI Genbank Gene ID: 219870). 24 h after transfection, the cells of all 7 transfections were harvested and pooled. 350 cells of this pool of cells with different OR genes inserted were then seeded into each well of seven 96-well plates.

3 days later, cells were stimulated with ligands for either OR5AN1 (10 μM musk ketone), OR10G7 (1 μM eugenol), or OR10G4 (100 μM vanitrope). One plate was treated with DMSO only. 6h after the addition of the OR ligands, puromycin was added to each plate at a final concentration of 1 μg/ml. On every day during the following week, the medium on all plates was replaced with fresh medium containing the corresponding ligand and puromycin. The plates were then searched for wells containing only a single colony of puromycin-resistant cells. The cells of those colonies were transferred to larger plates and grown until they had multiplied to about 8 Mio cells. The genomic DNA of 2 Mio cells of each clone were isolated using the Puregene kit according to the manufacturer's protocol. Using PCR primers 5′-ACAAGGACGACGACGATAAG-3′ (SEQ ID NO: 32) and 5′-GATGGCTGGCAACTAGAAGG-3′ (SEQ ID NO: 33), the OR gene stably integrated in the particular cell clone after the transfection with the pGL4.29-CRE-Puro-Hygro-OR plasmid was amplified and sequenced. The sequence alignments of the resulting sequences are shown below.

All 9 clones selected with musk ketone (Clone M1-M9, SEQ ID NO: 36-44) contained OR5AN1 which is known to respond to musks such as musk ketone, 8 out of 8 clones selected with eugenol (Clones E1-E8, SEQ ID NO: 45-53) contained OR10G7, a specific receptor for Eugenol, and 8 out of 8 colonies (Clone V1-V8, SEQ ID NO: 54-61) selected with vanitrope contained OR10G4, a receptor which responds to this ligand.

Thus, by applying the selection procedure of this invention, selective receptor de-orphanisation for a specific ligand is feasible as with this ligand it is possible to select from a pool of cells transfected with different ORs the particular cell expressing a receptor efficiently activated by the ligand of interest.

### Example 8. Generation of a Vector to Select for OR-Ligand Induced Blasticidin Resistance and Evaluation of Ligand-Induced Blasticidin Resistance in Stable Cell Lines Comprising Said Vector

To generate a vector allowing positive selection of cell clones with a functional expression of a specific OR gene, a blasticidin resistance gene was placed downstream of SEQ ID NO: 4. The pGL4.29-CRE-Puro-OR vector generated as described in Example 1 was digested with BgIII and FseI to remove the comprised basal promoter and the complete puromycin resistance gene. The removed basal promoter sequence was amplified with the primers AACATTTCTCTGGCCTAACTGG (SEQ ID NO: 28), GACAAAGGCATGGTGGCTTTACCAACAG (SEQ ID NO: 63), and the blasticidin S deaminase (bsd, SEQ ID NO: 64) encoding sequence was amplified by PCR using the primers GTAAAGCCACCATGCCTTTGTCTCAAGAAGAATCC (SEQ ID NO: 66) CCGACTCTAGATTAGCCCTCCCACACATAAC (SEQ ID NO: 67). The two overlapping PCR products were joined by fusion PCR using primers AACATTTCTCTGGCCTAACTGG (SEQ ID NO: 28) and ATCAGGCCGGCCGCCCCGACTCTAGATTAGCCCTCC (SEQ ID NO: 68). The resulting DNA fragment was digested with BgIII/FseI and ligated into the digested vector to generate pGL4.29-CRE-Blast-OR.

This vector was used to generate stable cell lines as described in Example 1.

Stable clones containing a stable insertion of the pGL4.29-CRE-BLAST-OR construct with the receptor OR5AN1 (NCBI Genbank Gene ID: 390195) were then plated at a density of 3000 cells/per well in 100 μl Dubelcco's Modified Eagle medium (DMEM) in polyethyleneimide-coated, white 96 well microtiter plates with a transparent bottom. After cells were allowed to adhere for 24 h, 50 μl of medium containing different odorants (musk ketone or ethyl vanillin at 40 μM) was added to each well resulting in an odorant concentration of 13.3 μM. 6 h after odorant addition, 50 μl of medium containing blasticidin was added to reach a final concentration of 0.5-256 μg/ml in 10 two-fold dilution steps (day 1 of selection). This step further diluted the odorant to 10 μM. On day 4 of the selection (72 h after blasticidin addition), medium from each well was removed completely, 100 μl PrestoBlue® cell viability reagent (ThermoFisher Scientific, Catalog No: 14200-083) diluted in phosphate buffered saline containing 1 mg/ml glucose was added to each well and cells were incubated at 37° C. until a color change was visible in the wells containing no blasticidin. The fluorescence was determined at 560 excitation and 590 nm emission to assess relative cell metabolic activity, which is the net result of cell growth and cell killing in presence of the different blasticidin concentrations. The result was expressed as % viability relative to controls with no cells (0%) and cells treated with olfactory ligand only (100%). Since cells were actively growing in this experiment a partial reduction in viability does not indicate cytotoxicity but can indicate (partial) cell stasis, while 0% viability indicates complete cytotoxicity.

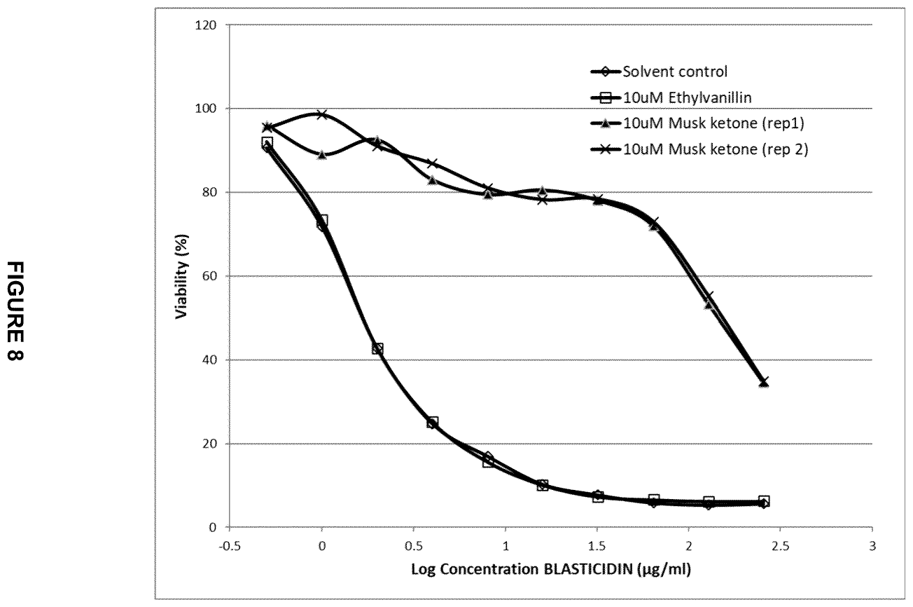

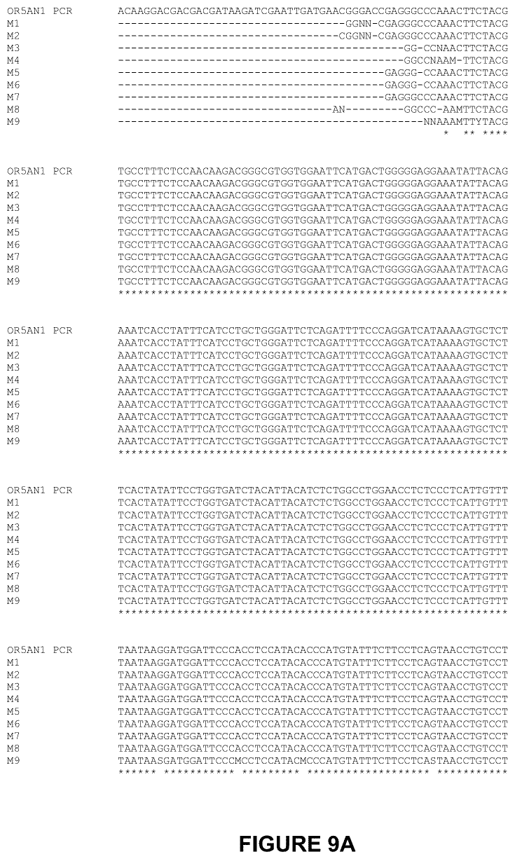

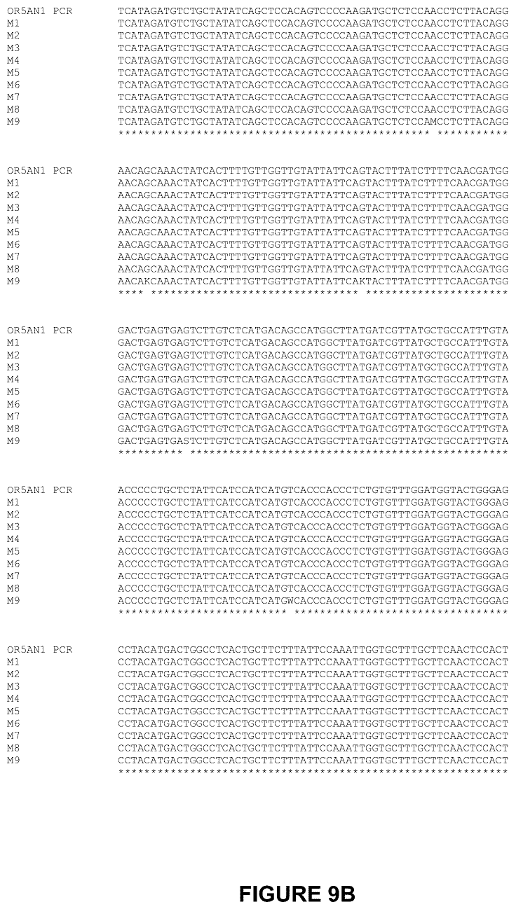

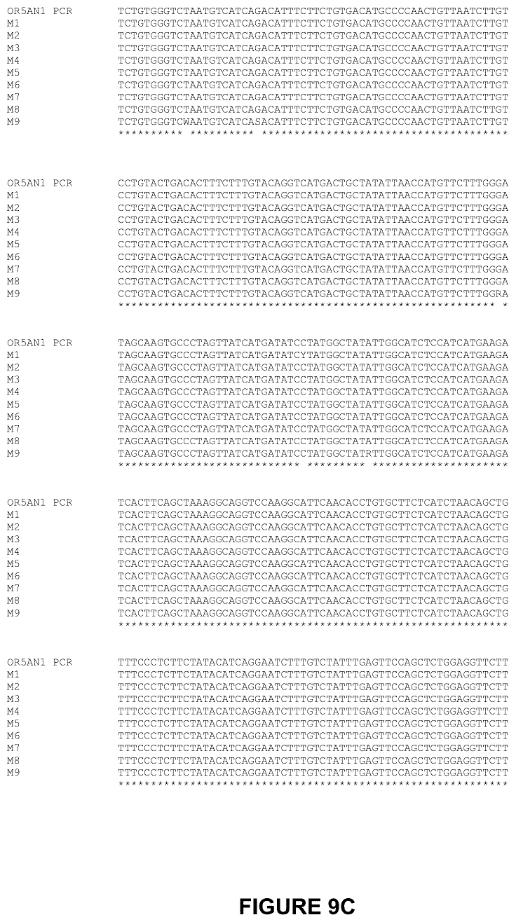

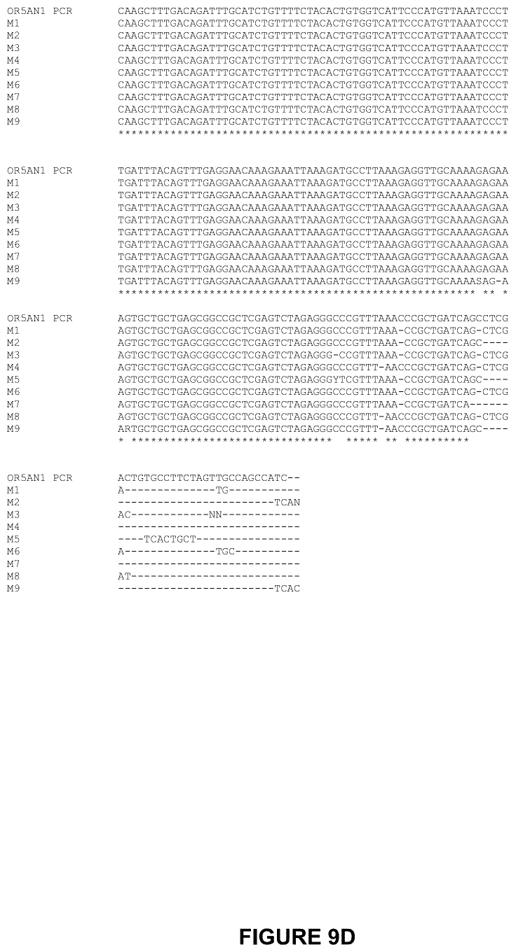

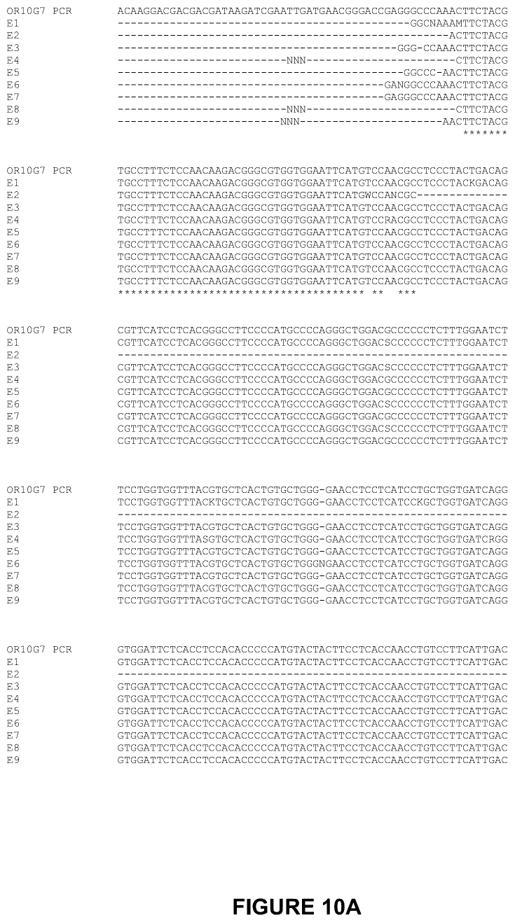

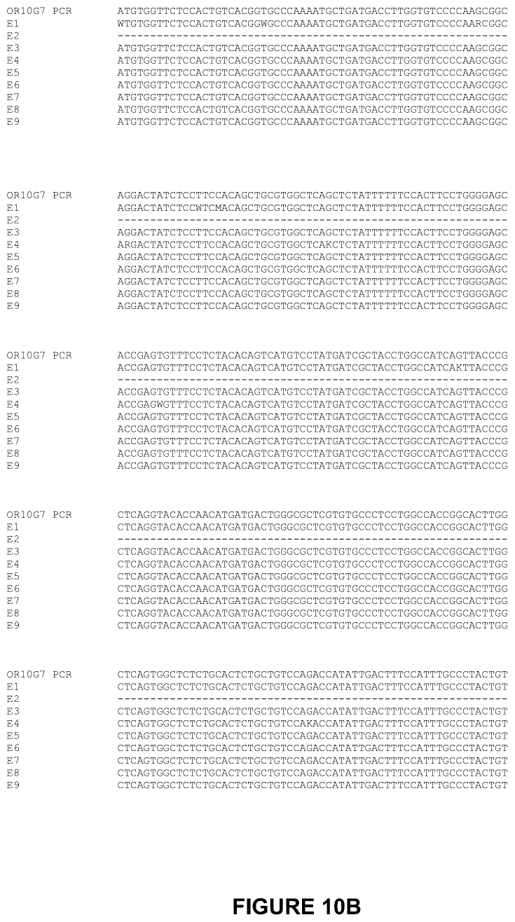

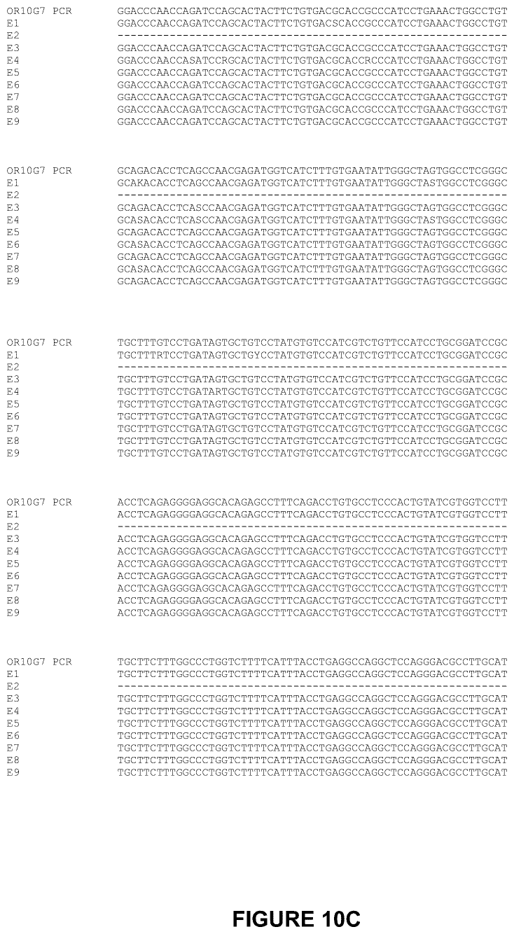

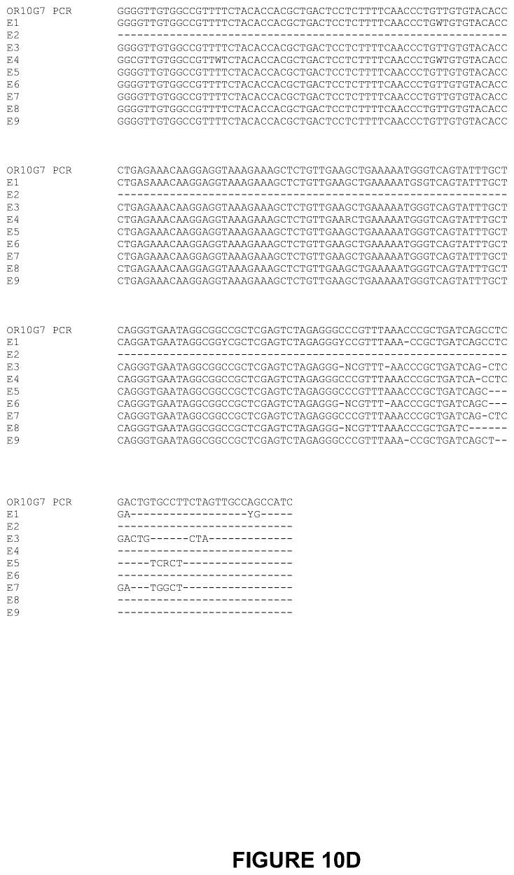

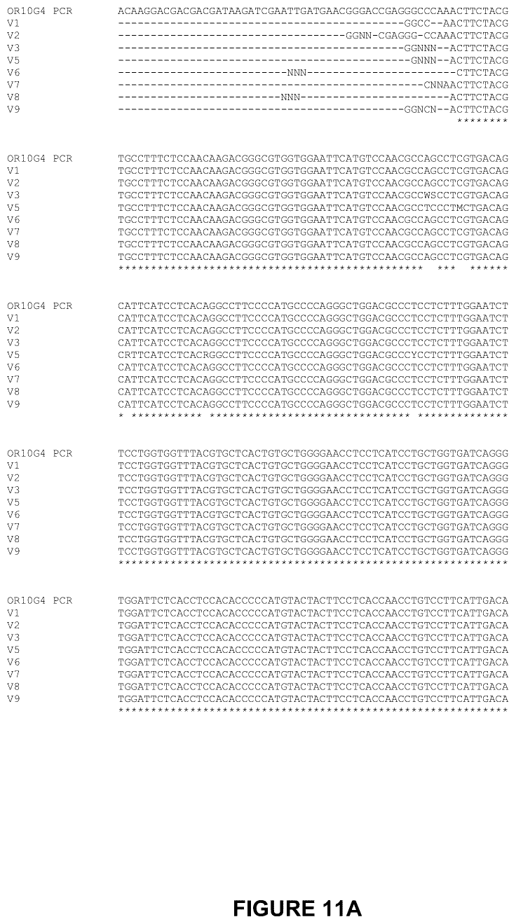

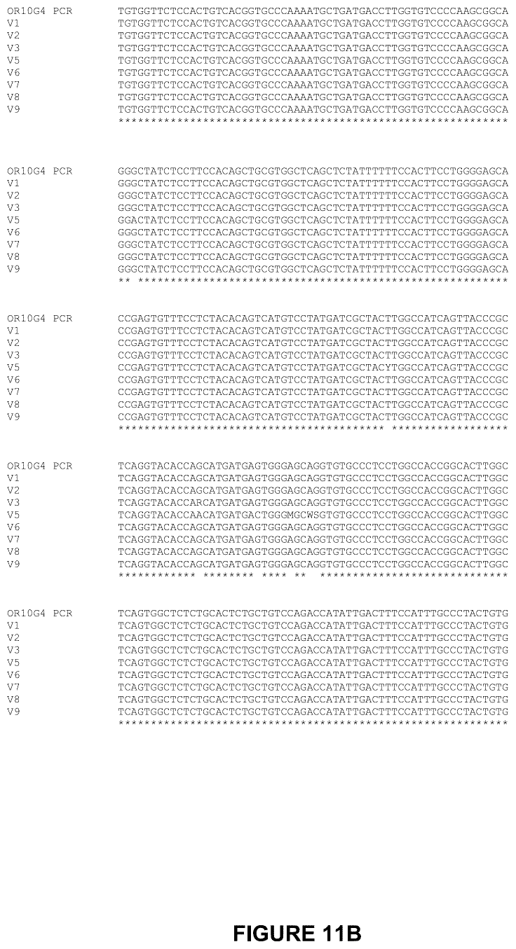

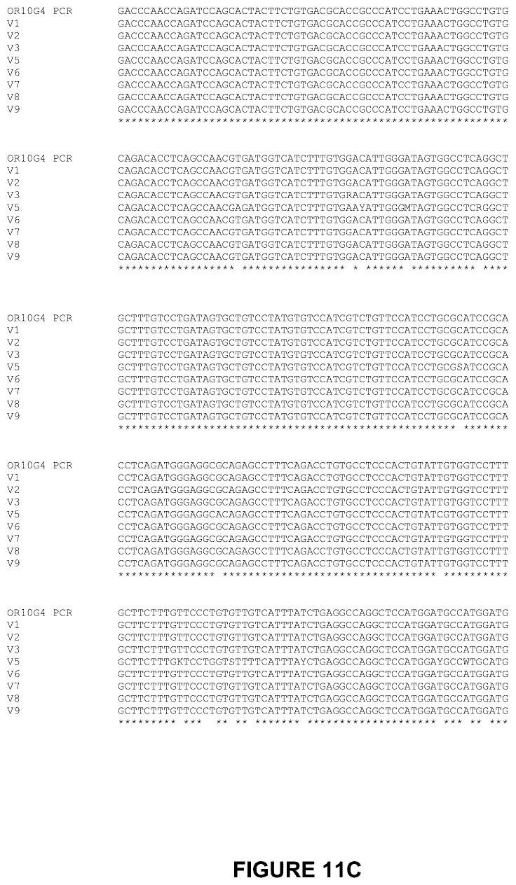

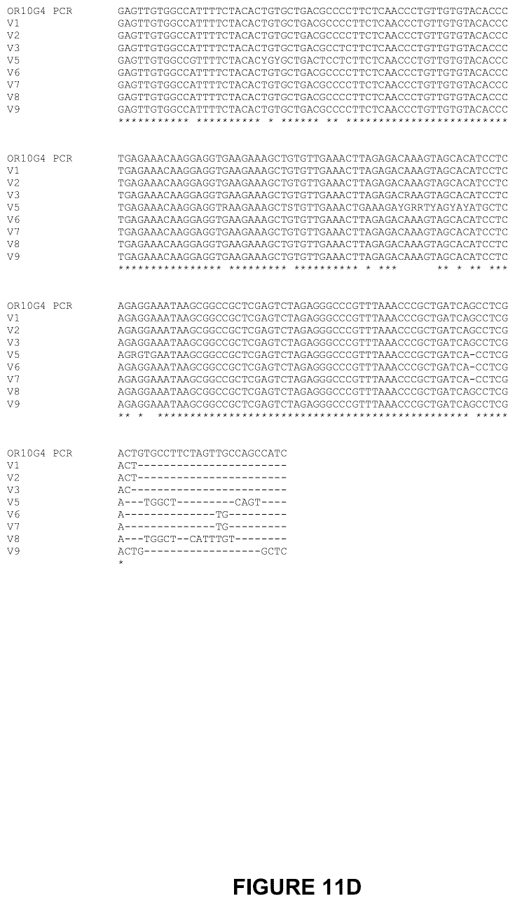

FIG. 8 shows the results for cells transformed with the vector pGL4.29-CRE-BLAST-OR containing the OR5AN1 gene, which is known to respond to musk compounds such as musk ketone. Enhanced blasticidin resistance and only minor reduction of viable cells was observed in presence of the musk ligand, while in presence of ethyl vanillin, which is not a ligand to this receptor, the cells were equally sensitive to blasticidin as in presence of the solvent control. Compared to puromycin, selection with blasticidin offers an even enhanced dynamic range, as e.g. the difference in viability at 32 μM is almost 10-fold (75% surviving cells in presence of the musk ligand, while only 7% viability is observed in absence of the ligand) and the concentration of blasticidin to reduce viability by 50% (EC50) is >50-fold higher in presence of the musk ligand.

1. A method for selecting for a cell expressing a functional olfactory receptor and/or for accessory molecules needed for said functional expression in a cell, said method comprising the following steps:
A) Providing cells, wherein said cells comprise a nucleic acid construct comprising a promoter and/or enhancer sequence operably linked to a nucleic acid sequence encoding a polypeptide, wherein said encoded polypeptide confers resistance to an antibiotic, wherein said promoter and/or enhancer is inducible by an olfactory receptor, wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor,
B) Culturing said cells in the presence of the ligand of said olfactory receptor,
C1) Selecting for cells functionally expressing the olfactory receptor by culturing them in the presence of the antibiotic and the ligand.

2. A method for selecting for a cell expressing a functional olfactory receptor and/or for accessory molecules needed for said functional expression in a cell, said method comprising the following steps:
A) Applying a mutagenesis step to cells, wherein said cells comprise a nucleic acid construct comprising a promoter and/or enhancer sequence operably linked to a nucleic acid sequence encoding a polypeptide, wherein said encoded polypeptide confers resistance to an antibiotic, wherein said promoter and/or enhancer is inducible by an olfactory receptor, wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor,
B) Culturing the mutated cells in the presence of the ligand of said olfactory receptor,
C1) Selecting for mutated cells functionally expressing the olfactory receptor by culturing them in the presence of the antibiotic and the ligand.

3. The method according to claim 2, wherein the mutagenesis step is carried out using insertional mutagenesis, wherein a nucleic acid sequence is inserted in the genome of the cells using plasmids, linearized DNA sequences, transposons, retroviruses, lentiviruses or CRISPR-Cas mediated recombination.

4. The method according to claim 3, wherein said inserted nucleic acid sequence comprises an enhancer and/or promoter sequence suitable for activation of expression of endogenous genes.

5. The method according to claim 3, wherein the insertion site of the inserted nucleic acid sequence in the selected cells is mapped and/or identified.

6. The method according to claim 2, wherein the mutagenesis step is carried out using CRISPR-Cas-mediated mutagenesis, using CRISPR interference or CRISPR activation.

7. A method for identifying an olfactory receptor binding to a given ligand, said method comprising the following steps:
A) Providing a heterogeneous population of cells, wherein said cells comprise a nucleic acid construct comprising a promoter and/or enhancer sequence operably linked to a nucleic acid sequence encoding a polypeptide, wherein said encoded polypeptide confers resistance to an antibiotic, wherein said promoter and/or enhancer is inducible by an olfactory receptor, wherein said promoter and/or enhancer sequence comprises one or more copies of a cAMP responsive element (CRE), a half CRE, or an NFAT responsive element (NFAT-RE), and a second nucleic acid construct comprising a nucleic acid molecule encoding an olfactory receptor, wherein the olfactory receptor encoded by the nucleic acid molecule comprised in said second nucleic acid construct comprised in at least one of the cells is distinct from the olfactory receptor encoded by the nucleic acid molecule comprised in the second nucleic acid construct in at least one of the other cells of said population,
B) Culturing said population of cells in the presence of said given ligand,
C1) Selecting for cells functionally expressing the olfactory receptor binding to said given ligand by culturing them in the presence of the antibiotic and the ligand.
D) Determining the nucleotide sequence encoding the receptor in the selected cells.

8. The method according to claim 1, wherein step C1) additionally comprises a sub-culturing step, wherein cells with improved functional expression of the olfactory receptor are enriched in a culture.

9. The method according to claim 1, wherein the nucleic acid sequence encoding the polypeptide that confers resistance to an antibiotic is a puromycin-N-acetyltransferase gene or a blasticidin-S deaminase gene.

10. (canceled)

11. (canceled)

12. (canceled)

13. (canceled)

14. (canceled)

15. (canceled)

16. (canceled)

17. (canceled)
